SEQUENCE LISTING (23 sequences)

SEQ ID NO: 1 - DNA, 1245 bp, synthetic construct
  source 1..1245: mol_type = other DNA
  misc_feature 1..1245: note = Phytase gene
agcgaaaccgaaccttccggatatcagctggagaaggtggtcattctctcgcgtcacggtgtccgagcccccaccaagatgacacagacgatgcgcgatgtcactcctcatcagtggcctgagtggcccgtgaagctcggctacatcactcctcgtggagaacacctcatcagcctgatgggcggtttctatagggaacggttccagcagcagggattgcttcccaacgacacctgtccgacccccgacgccgtctacgtgtggaccgacgttaaccagcgtacccgcaagactggagaggctttcctcgccggtcttgcgcctcagtgtgatctggccatccaccaccagcagaacatcacgcaggctgacccgctgtttcacccggtcaaggccggtatctgttcgatgaacaagtctcagacctatgcggctgtcgagaagcaggctggcggccctattgagacgctaaaccagcgctaccaggccgaactggcattgatgtcctctgtgttggatttccccaagtccccatattgccagcagcataacatcggcaaactgtgcgacttttcacaggctatgcctagccgcctcaacatctccgatgacgggaatgaggtgcaactcgaaggcgccgtcggtctttcctccacgctcgccgagatcttcctactggaatacgctcagggtatgcctgtggtcgcctggggcaacattcacaacgagagccagtggaagagcctccttaacttgcacaacgcccatttcaacctgatgcacagaacgccctacattgccaagcaccagggaacccctttacttcaggctatcagcaacgctctcaacccaaatgcaactgagtcgaagctccccgatatctctcccgacaacaagatccttttcattgccggccacgacaccaacatcgcaaacatcggaggcatgttgggtatgaactggactctcccgggccagccagacaatactccgcccggcggtggactggttttcgaactctggcagaacccggataaccatcagcagtacgttgcggtgaagatgatctaccagaccatggaccagctgcgcaattccgagaagctggacttgaagagcaaccctgctgggatcgtccccattgagatcgaaggttgcgagaacatcggtaccgacaagctgtgccagctggatacttttcagaagcgtgttgcccaggtcattgagcccgcgtgccaaatctaa

SEQ ID NO: 2 - protein, 414 aa, synthetic construct
  REGION 1..414: note = Phytase PhV-99
SETEPSGYQLEKVVILSRHGVRAPTKMTQTMRDVTPHQWPEWPVKLGYITPRGEHLISLMGGFYRERFQQQGLLPNDTCPTPDAVYVWTDVNQRTRKTGEAFLAGLAPQCDLAIHHQQNITQADPLFHPVKAGICSMNKSQTYAAVEKQAGGPIETLNQRYQAELALMSSVLDFPKSPYCQQHNIGKLCDFSQAMPSRLNISDDGNEVQLEGAVGLSSTLAEIFLLEYAQGMPVVAWGNIHNESQWKSLLNLHNAHFNLMHRTPYIAKHQGTPLLQAISNALNPNATESKLPDISPDNKILFIAGHDTNIANIGGMLGMNWTLPGQPDNTPPGGGLVFELWQNPDNHQQYVAVKMIYQTMDQLRNSEKLDLKSNPAGIVPIEIEGCENIGTDKLCQLDTFQKRVAQVIEPACQI

SEQ ID NO: 3 - DNA, 7599 bp, synthetic construct
  source 1..7599: mol_type = other DNA
  misc_feature 1..7599: note = pMT940
tgaagaagggcgtaaataagaagctataatagcttaaaggtataattacctaacttagtaagcatactatattagccactatactataaagggctcctagaacgtaactacctatagcttagctaccactagctactataagggctagcgcccctcctagcttaatcgcctacctagtcgtatacctaccgcctacgactactagctagaaacgactacggctagaaacgctagcataggagagcccctcacgtaagagggctactagcgcgctctatagtaaccgtaccctagccagggacacctaggccagagctaggactctcgtacacctacagtaggagcgcccgtcagcactatccgtattcctagacaacctaggcctggctagtagtaagaaggactgacggataagcggatagatacgaccgtaggatagaggaacgattgtaaggacggtcataatagataaaagactatatttactaagttataacgggtcgtcatttttccttttgaaccctggtacctattatagcatttggctatctataggttagagctaactaacttactaatagggataataagctagggtcaatatatatttagatagcttctctaagtgcctctagtatttagaacaattaacgcttttattacctacttctgtagaccgtaacaattagtctaggagtgccgcttatagcgcgaatactgctgtggtatcatacctttacagcctctctaagctgtactgattatagccaccgagctatagcacaaatagctcgcttgacttggactttgatcaactacgatgcgaaaaagaaaagcgagaggaaaatgggaagaaaaaaatttttttaaaaaaaatgaaccatataatttaattagcaccgggattggctgttcgttgcctcttctagcttggttggggagagcggacggaatcccaaaatttccggcacgtatggtcgtttgtaattgggtctaccaaaagttgattagtagtgctcgcctgttgaagttaaggcgcctagaggcatataatattactaataatagtccttacactcttggggtccataaaccgtgtatccctgcgccgatgaatgtgctctccatttgagcaaatagaatagtcgacagcaccacgaattaacaggataaagtacggtatgatgggtcagttcagcaatgtgtgagtagtgtactgtggtaagaaggtaggcggtagagagcgggctctccagaccacagaaagagggctccgtcccacaaaggcccagacccacttccaggggtatttatctcaactacatgcaactgggacatacagacaaacgcgaactacgttaaagtgagaactgaagaggtcaaggcatcaagatggttcacatggtctgaaaagaagccattttgttacgtgaaaaaaaaggtttatgtactggtggagcgaagcactccagagtgaaaaataaaactatatacagaagaatcgcgcgtgtctcagaacctccttcagagaggttcgtcgtaataatgggcgctttctactctggacttgcctctttgtgctttacattgggggtcgagcctttccaatcagaagatgcacttaccaaagtagcgctaggctacacagcgctttacccccgaatcaggcgtggggtcactaccccgacgcccttttttttttttttttttgtattcacagagcggtttgcacaatctagatttgttcctcccgcctagtcggtcgccttgtacttggcccggtactcctccgtcctcctgatgtcctcctcggatgcgagccccttgataccctcgatgatgtcgtccaaggtgagaatcgcaaagatgggcagaccatactccttccggagctcaccgatcgcgctcggcatcggcttgctatcgtcgccatccggcgaaggcagcttctccatgcggtccagcgcgaccacgatgccggctacgataccgccctccttctcaatcttggcgatcgcctcacgcttcgccgtgccagccgtgatcacatcatccacgataagcaccctcttgcccttgagcggcgcccccacaatgttgccgccctcaccgtggtcctttgcctccttgcggtcgaacgagtagcacgtggtatggccgtacgtggccggatccagctggcccagccgaatggtggccgccgtcgccaacgggatccccttgtaggccgggccgaagacgatgtcgaactggatcgcgccgctccggtgcgcctcgatgatggcgtgcgcgtacgccgtcgcgagcgcctcgagcagatcggcccggtagaagtctcccgcgttgaagaagtagggcgagatgcgcttcgacttgagctcgaagctgccaaacttgaggatgttgccggcgatggccgcccggaggaagtcggctttgtaagaagcgagtggggccatgtctgtcggttcgtacgtgtcttacgggttcttgggctggaagtttatttgttaccggagggaactcctggtgaagattcgaaaaaaagaaatgagggagttttgtcaaacgtccccctatcgcagagctgaagagagcccctccatcgcgaatttcttggtggggttggattcatggtggggcccaagggtcgttaaacttttttgtatgtctacggagttacacagtactccgtacgtcgtaccccacttgcaatgcttctcgagctgggcttgggatattttgataacgcacaccgcctgccttatcttatcattttgtgctctccgcaaaaaattatttcgggcgacttggggacggcaaatcgacaccgccccgccaggtgctccctatctgacaactcccaaacacccccatcgccaaattcggacagcaccctgcgcgttgcctgccatggcctggaaggatatcgcgcctgtgcctacgtaagggttcaattcgtatatatgctgttcctttcctgaactaagctgatcattttcgtacagggcacaggagtttatcgacatcatcctttcgtaagtttgaaggctaggttatacgtagcggccgctcgatgagacctttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagttccagtgagtcgtattacgcgcgctcactggccgtcgttttacaacgtcgtgactgggaaaaccctggcgttacccaacttaatcgccttgcagcacatccccctttcgccagttggcgtaatagcgaagaggcccgcacagatcgcccttcccaacagttgcgcagcctgaatggcgaatggcgtctgatgcggtattttctccttacgcatctgtgcggtatttcacaccgcatctggtgcactctcagtacaatctgctctgatgccgcatagtaaagccagccccgacacccgccaacacccgctgacgcgccctgacgggcttgtctgctaccaataaaacgaaaggctcagtcgaaagactgggcctttcgttttatccggtctattcttttgttcgccctattttataagggattttgccgatttcggcctattgggtctctgatctacgtacaagacgagtgtgccagtcagccatcgaatgatgggtcgtcatggacaacggagcaatgccatctcgggactggggccaccgacctgccggcagggggaggagaaaaaaaagggggcggcgtcggcaagatggcaagaggtctgggagatagtcaaggacgaagggccaagcgggtcgtgtcgcttgcactctggtgaggctcgctagtgcttgcggtgggtgaggtggatcagagtagaggcccgatggagaatgggcgctgcggtgacaagcctaaccggtcgatagcggctagtaggctgggcggcgatgtcacgttatggttgccacggccgggcacggactgagggcatcgttcgaggacgtggagccgtgcgatggaagcgattaaggttgctgaagggtcggcgagggcaaccacacacctcattggaggagtctgggcgcggcagccaggttatcaggggtgacacggaaggcgtccgccgagtgcgactctgcgaagcccaggccgccctgcggaagaaccttggttcacgatgctctctgatgaccgcagaagagcgtatttgacgctccgcgcaaatgcgatccgaagtgccggtgcgaggctcctgtcgcctgctcgtctccgccctgactctcaacgcagcacgaacagcgctgccgtagatactccttcggagcctaccctggtgtacactagattcaagcaaaaggcgcacgagagcggagaaagccgcagcacaccccatcccttcccttcgcaacggatctcgcttctccccgcattttagtgtagtcccggtggcccacttaccccggcttctgcagtcttggtatttggtggtttctgatgtgcggggagagaggccttcagaaaaaaatgtgtctcttcgtggaactcgccgtgtgtgcgactttaacttgtatggaagttgaggtcgatttccccagatcttgaggctcaaaaactcccaaatttggcaccaaacgactgggacgcaaaaccgggtacctggtgccatcttcaatatacttgggaatgcagccgcggtgattgtcggtctgggcgcaacctcatcggcgttggcgggcgatcagcaggtgcaacggggggtgcaaaccgggaaatgagaagagcccagaccaacgaggaccgcggcatcgacctccgatcaccctaagttgggcctagtgccgccccaccaaggaacggaccagggtagtttgctcctcccgatcactggtagtcgtgccacagtgcagcgcattcaagggcaagccgctttcccaaatagagcaggaagggctggtttcaactttgaggtcaggacatccgtaacgttattccactttcggtgttctctttcttccgcagcttcttcggcagcgagttagttgcgcaactctcgtcacttttgcaatactttcatcgacgttccccaaatcggatacctcatcgatgctcttctgcgttaattaacaattgggatcctctagacccgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgctcgatttaaatctcgagaggcctgacgtcgggcccggtaccacgcgtcatatgactag

SEQ ID NO: 4 - DNA, 10031 bp, synthetic construct
  source 1..10031: mol_type = other DNA
  misc_feature 1..10031: note = pMT2194
gaagaagggcgtaaataagaagctataatagcttaaaggtataattacctaacttagtaagcatactatattagccactatactataaagggctcctagaacgtaactacctatagcttagctaccactagctactataagggctagcgcccctcctagcttaatcgcctacctagtcgtatacctaccgcctacgactactagctagaaacgactacggctagaaacgctagcataggagagcccctcacgtaagagggctactagcgcgctctatagtaaccgtaccctagccagggacacctaggccagagctaggactctcgtacacctacagtaggagcgcccgtcagcactatccgtattcctagacaacctaggcctggctagtagtaagaaggactgacggataagcggatagatacgaccgtaggatagaggaacgattgtaaggacggtcataatagataaaagactatatttactaagttataacgggtcgtcatttttccttttgaaccctggtacctattatagcatttggctatctataggttagagctaactaacttactaatagggataataagctagggtcaatatatatttagatagcttctctaagtgcctctagtatttagaacaattaacgcttttattacctacttctgtagaccgtaacaattagtctaggagtgccgcttatagcgcgaatactgctgtggtatcatacctttacagcctctctaagctgtactgattatagccaccgagctatagcacaaatagctcgcttgacttggactttgatcaactacgatgcgaaaaagaaaagcgagaggaaaatgggaagaaaaaaatttttttaaaaaaaatgaaccatataatttaattagcaccgggattggctgttcgttgcctcttctagcttggttggggagagcggacggaatcccaaaatttccggcacgtatggtcgtttgtaattgggtctaccaaaagttgattagtagtgctcgcctgttgaagttaaggcgcctagaggcatataatattactaataatagtccttacactcttggggtccataaaccgtgtatccctgcgccgatgaatgtgctctccatttgagcaaatagaatagtcgacagcaccacgaattaacaggataaagtacggtatgatgggtcagttcagcaatgtgtgagtagtgtactgtggtaagaaggtaggcggtagagagcgggctctccagaccacagaaagagggctccgtcccacaaaggcccagacccacttccaggggtatttatctcaactacatgcaactgggacatacagacaaacgcgaactacgttaaagtgagaactgaagaggtcaaggcatcaagatggttcacatggtctgaaaagaagccattttgttacgtgaaaaaaaaggtttatgtactggtggagcgaagcactccagagtgaaaaataaaactatatacagaagaatcgcgcgtgtctcagaacctccttcagagaggttcgtcgtaataatgggcgctttctactctggacttgcctctttgtgctttacattgggggtcgagcctttccaatcagaagatgcacttaccaaagtagcgctaggctacacagcgctttacccccgaatcaggcgtggggtcactaccccgacgcccttttttttttttttttttgtattcacagagcggtttgcacaatctagatttgttcctcccgcctagtcggtcgccttgtacttggcccggtactcctccgtcctcctgatgtcctcctcggatgcgagccccttgataccctcgatgatgtcgtccaaggtgagaatcgcaaagatgggcagaccatactccttccggagctcaccgatcgcgctcggcatcggcttgctatcgtcgccatccggcgaaggcagcttctccatgcggtccagcgcgaccacgatgccggctacgataccgccctccttctcaatcttggcgatcgcctcacgcttcgccgtgccagccgtgatcacatcatccacgataagcaccctcttgcccttgagcggcgcccccacaatgttgccgccctcaccgtggtcctttgcctccttgcggtcgaacgagtagcacgtggtatggccgtacgtggccggatccagctggcccagccgaatggtggccgccgtcgccaacgggatccccttgtaggccgggccgaagacgatgtcgaactggatcgcgccgctccggtgcgcctcgatgatggcgtgcgcgtacgccgtcgcgagcgcctcgagcagatcggcccggtagaagtctcccgcgttgaagaagtagggcgagatgcgcttcgacttgagctcgaagctgccaaacttgaggatgttgccggcgatggccgcccggaggaagtcggctttgtaagaagcgagtggggccatgtctgtcggttcgtacgtgtcttacgggttcttgggctggaagtttatttgttaccggagggaactcctggtgaagattcgaaaaaaagaaatgagggagttttgtcaaacgtccccctatcgcagagctgaagagagcccctccatcgcgaatttcttggtggggttggattcatggtggggcccaagggtcgttaaacttttttgtatgtctacggagttacacagtactccgtacgtcgtaccccacttgcaatgcttctcgagctgggcttgggatattttgataacgcacaccgcctgccttatcttatcattttgtgctctccgcaaaaaattatttcgggcgacttggggacggcaaatcgacaccgccccgccaggtgctccctatctgacaactcccaaacacccccatcgccaaattcggacagcaccctgcgcgttgcctgccatggcctggaaggatatcgcgcctgtgcctacgtaagggttcaattcgtatatatgctgttcctttcctgaactaagctgatcattttcgtacagggcacaggagtttatcgacatcatcctttcgtaagtttgaaggctaggttatacgtagcggccgctcgacgatgcgtctcgtggaaagttaggcacctcccccctttcggggcgagtagccgaagcgggggcatcaactgattaacaggcttgttaaaggaagtcttcacggtcggcatacaaatcggccaagacgttgaagatgccataatacataatatgtatgtacaataaccgggcacatacggtgcaactgctagtcagcaattgcgcctcgctttttgacacagagggcaatgcagaacgtcgaatcgaccgagtcagttgctgttggcgtcatgatggctctagggtcagacagaaggtcagagaaagcatgccttagctcgaagccgctcagatgtaattacgcaacgctcggcgttcgagtttacggaggacgacggctacaagatggggctgcttaaagttaccttaaatagaaaatagtgcctggcttaagagatcatgtccgcggggctagcaaggatgtcgggtcttaactcgacggctcgcctagatttcgtgaaaagggaactcactccccgacaggcccgcaagtgaatatgtaattactcaatggaagttctcgaaacggagtccagaaatgatgtggttctgtgggaatgcggcaagaggcgacgttgccgtgaatgcgtgaacattcccgcctcttcttcttctcgtcttcttccttcttcttctttcgggtcgcggatggttgacggccagcgtgcgcacggctgcgtgttatcgagcgtcggtacgtctagccaacatcccgtagacacgacgaccaagcgtcttgagaatgcaacaacgtctcggaacctggcacgcatcttccgccgcaggtcggcagacgccgcctgggcaataccacccctgtccaggccctttccccgcaggcagagccgcgctcttcctttcatggttattcaggaacgtggcttccgagattctcgcctgttctcccccagtcaacctgccgaccgtaacccggttccaccaccgcggactgtccgcaaaacctggttcgcccgagattaatatgctatttccggactaagtgcacaacacacaagcaccccttccgcctcgcgctctagaatctgctttctaacccggttctcgggcccttccctttcgcgacgcctccgctctccttaccaggcaccatccgcaataggtaaggtagccaaccgttttggagcgtgattctgccaaggaccgcatccttgcattcgccatctggtcaaggacccctctttcccgctccattctggtggctctatcgggacggcgttccccatggctctccaggagagtgatgtgcgagtctggagagccggggttggcgtcacgatgctgcccacctagggccggccagcccggcactgcgctcccgttgatccgtctatccccgtcaagagcaccagccccggcgctcgtgaattttcgacttgttcgacttgctacaggtgataaagaggatgcacgccgccctcgatcggcctgtgtggtttctctccctcgtgccaaaccactcccacctcccgccccgagatagttgcttgtttcgctccgtgagagggacacacacccatggtgactccctctctgaagaaggcagctctggctgccctgtccctcttcccgctcctctccctggctagcgaaaccgaaccttccggatatcagctggagaaggtggtcattctctcgcgtcacggtgtccgagcccccaccaagatgacacagacgatgcgcgatgtcactcctcatcagtggcctgagtggcccgtgaagctcggctacatcactcctcgtggagaacacctcatcagcctgatgggcggtttctatagggaacggttccagcagcagggattgcttcccaacgacacctgtccgacccccgacgccgtctacgtgtggaccgacgttaaccagcgtacccgcaagactggagaggctttcctcgccggtcttgcgcctcagtgtgatctggccatccaccaccagcagaacatcacgcaggctgacccgctgtttcacccggtcaaggccggtatctgttcgatgaacaagtctcagacctatgcggctgtcgagaagcaggctggcggccctattgagacgctaaaccagcgctaccaggccgaactggcattgatgtcctctgtgttggatttccccaagtccccatattgccagcagcataacatcggcaaactgtgcgacttttcacaggctatgcctagccgcctcaacatctccgatgacgggaatgaggtgcaactcgaaggcgccgtcggtctttcctccacgctcgccgagatcttcctactggaatacgctcagggtatgcctgtggtcgcctggggcaacattcacaacgagagccagtggaagagcctccttaacttgcacaacgcccatttcaacctgatgcacagaacgccctacattgccaagcaccagggaacccctttacttcaggctatcagcaacgctctcaacccaaatgcaactgagtcgaagctccccgatatctctcccgacaacaagatccttttcattgccggccacgacaccaacatcgcaaacatcggaggcatgttgggtatgaactggactctcccgggccagccagacaatactccgcccggcggtggactggttttcgaactctggcagaacccggataaccatcagcagtacgttgcggtgaagatgatctaccagaccatggaccagctgcgcaattccgagaagctggacttgaagagcaaccctgctgggatcgtccccattgagatcgaaggttgcgagaacatcggtaccgacaagctgtgccagctggatacttttcagaagcgtgttgcccaggtcattgagcccgcgtgccaaatctaatagcacgaacctctctgaaggaggttctgagacacgcgcgattcttctgtatatagttttatttttcactctggagtgcttcgctccaccagtacataaacctttttttcacgtaacaaaatggcttcttttcagaccatgtgaaccatcttgatgccttgacctcttcagttctcactttaacgtagttcgcgtttgtctgtatgtcccagatctacgtacaagacgagtgtgccagtcagccatcgaatgatgggtcgtcatggacaacggagcaatgccatctcgggactggggccaccgacctgccggcagggggaggagaaaaaaaagggggcggcgtcggcaagatggcaagaggtctgggagatagtcaaggacgaagggccaagcgggtcgtgtcgcttgcactctggtgaggctcgctagtgcttgcggtgggtgaggtggatcagagtagaggcccgatggagaatgggcgctgcggtgacaagcctaaccggtcgatagcggctagtaggctgggcggcgatgtcacgttatggttgccacggccgggcacggactgagggcatcgttcgaggacgtggagccgtgcgatggaagcgattaaggttgctgaagggtcggcgagggcaaccacacacctcattggaggagtctgggcgcggcagccaggttatcaggggtgacacggaaggcgtccgccgagtgcgactctgcgaagcccaggccgccctgcggaagaaccttggttcacgatgctctctgatgaccgcagaagagcgtatttgacgctccgcgcaaatgcgatccgaagtgccggtgcgaggctcctgtcgcctgctcgtctccgccctgactctcaacgcagcacgaacagcgctgccgtagatactccttcggagcctaccctggtgtacactagattcaagcaaaaggcgcacgagagcggagaaagccgcagcacaccccatcccttcccttcgcaacggatctcgcttctccccgcattttagtgtagtcccggtggcccacttaccccggcttctgcagtcttggtatttggtggtttctgatgtgcggggagagaggccttcagaaaaaaatgtgtctcttcgtggaactcgccgtgtgtgcgactttaacttgtatggaagttgaggtcgatttccccagatcttgaggctcaaaaactcccaaatttggcaccaaacgactgggacgcaaaaccgggtacctggtgccatcttcaatatacttgggaatgcagccgcggtgattgtcggtctgggcgcaacctcatcggcgttggcgggcgatcagcaggtgcaacggggggtgcaaaccgggaaatgagaagagcccagaccaacgaggaccgcggcatcgacctccgatcaccctaagttgggcctagtgccgccccaccaaggaacggaccagggtagtttgctcctcccgatcactggtagtcgtgccacagtgcagcgcattcaagggcaagccgctttcccaaatagagcaggaagggctggtttcaactttgaggtcaggacatccgtaacgttattccactttcggtgttctctttcttccgcagcttcttcggcagcgagttagttgcgcaactctcgtcacttttgcaatactttcatcgacgttccccaaatcggatacctcatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgctcgatttaaatctcgagaggcctgacgtcgggcccggtaccacgcgtcatatgactagt

SEQ ID NO: 5 - DNA, 9634 bp, synthetic construct
  source 1..9634: mol_type = other DNA
  misc_feature 1..9634: note = pMT2195
gaagaagggcgtaaataagaagctataatagcttaaaggtataattacctaacttagtaagcatactatattagccactatactataaagggctcctagaacgtaactacctatagcttagctaccactagctactataagggctagcgcccctcctagcttaatcgcctacctagtcgtatacctaccgcctacgactactagctagaaacgactacggctagaaacgctagcataggagagcccctcacgtaagagggctactagcgcgctctatagtaaccgtaccctagccagggacacctaggccagagctaggactctcgtacacctacagtaggagcgcccgtcagcactatccgtattcctagacaacctaggcctggctagtagtaagaaggactgacggataagcggatagatacgaccgtaggatagaggaacgattgtaaggacggtcataatagataaaagactatatttactaagttataacgggtcgtcatttttccttttgaaccctggtacctattatagcatttggctatctataggttagagctaactaacttactaatagggataataagctagggtcaatatatatttagatagcttctctaagtgcctctagtatttagaacaattaacgcttttattacctacttctgtagaccgtaacaattagtctaggagtgccgcttatagcgcgaatactgctgtggtatcatacctttacagcctctctaagctgtactgattatagccaccgagctatagcacaaatagctcgcttgacttggactttgatcaactacgatgcgaaaaagaaaagcgagaggaaaatgggaagaaaaaaatttttttaaaaaaaatgaaccatataatttaattagcaccgggattggctgttcgttgcctcttctagcttggttggggagagcggacggaatcccaaaatttccggcacgtatggtcgtttgtaattgggtctaccaaaagttgattagtagtgctcgcctgttgaagttaaggcgcctagaggcatataatattactaataatagtccttacactcttggggtccataaaccgtgtatccctgcgccgatgaatgtgctctccatttgagcaaatagaatagtcgacagcaccacgaattaacaggataaagtacggtatgatgggtcagttcagcaatgtgtgagtagtgtactgtggtaagaaggtaggcggtagagagcgggctctccagaccacagaaagagggctccgtcccacaaaggcccagacccacttccaggggtatttatctcaactacatgcaactgggacatacagacaaacgcgaactacgttaaagtgagaactgaagaggtcaaggcatcaagatggttcacatggtctgaaaagaagccattttgttacgtgaaaaaaaaggtttatgtactggtggagcgaagcactccagagtgaaaaataaaactatatacagaagaatcgcgcgtgtctcagaacctccttcagagaggttcgtcgtaataatgggcgctttctactctggacttgcctctttgtgctttacattgggggtcgagcctttccaatcagaagatgcacttaccaaagtagcgctaggctacacagcgctttacccccgaatcaggcgtggggtcactaccccgacgcccttttttttttttttttttgtattcacagagcggtttgcacaatctagatttgttcctcccgcctagtcggtcgccttgtacttggcccggtactcctccgtcctcctgatgtcctcctcggatgcgagccccttgataccctcgatgatgtcgtccaaggtgagaatcgcaaagatgggcagaccatactccttccggagctcaccgatcgcgctcggcatcggcttgctatcgtcgccatccggcgaaggcagcttctccatgcggtccagcgcgaccacgatgccggctacgataccgccctccttctcaatcttggcgatcgcctcacgcttcgccgtgccagccgtgatcacatcatccacgataagcaccctcttgcccttgagcggcgcccccacaatgttgccgccctcaccgtggtcctttgcctccttgcggtcgaacgagtagcacgtggtatggccgtacgtggccggatccagctggcccagccgaatggtggccgccgtcgccaacgggatccccttgtaggccgggccgaagacgatgtcgaactggatcgcgccgctccggtgcgcctcgatgatggcgtgcgcgtacgccgtcgcgagcgcctcgagcagatcggcccggtagaagtctcccgcgttgaagaagtagggcgagatgcgcttcgacttgagctcgaagctgccaaacttgaggatgttgccggcgatggccgcccggaggaagtcggctttgtaagaagcgagtggggccatgtctgtcggttcgtacgtgtcttacgggttcttgggctggaagtttatttgttaccggagggaactcctggtgaagattcgaaaaaaagaaatgagggagttttgtcaaacgtccccctatcgcagagctgaagagagcccctccatcgcgaatttcttggtggggttggattcatggtggggcccaagggtcgttaaacttttttgtatgtctacggagttacacagtactccgtacgtcgtaccccacttgcaatgcttctcgagctgggcttgggatattttgataacgcacaccgcctgccttatcttatcattttgtgctctccgcaaaaaattatttcgggcgacttggggacggcaaatcgacaccgccccgccaggtgctccctatctgacaactcccaaacacccccatcgccaaattcggacagcaccctgcgcgttgcctgccatggcctggaaggatatcgcgcctgtgcctacgtaagggttcaattcgtatatatgctgttcctttcctgaactaagctgatcattttcgtacagggcacaggagtttatcgacatcatcctttcgtaagtttgaaggctaggttatacgtagcggccgctcgaggatacgagctctctcggggacgaatagatgaaggcaatctgacagatttgctctcaaaaaaagactgatatctcttccaccatgcactgtatgtacattacatacattatccccctccactggattcgcacaacggaaagcaatggcgcgctgattcaagaaccatcagggctgtcattggcttgttttgtgccgtggccgcggtgacgcccactatgactctctgggcaggcggcaactgggtgccagatatattaatccggggcatagcgcatatcttccttgatttgtagagtactagtacactaacccccttctccacatggggccactgttcggtagatctgcccgaagtgcaagtgcggggggggccaaactaggtaatatcctcccgctctcccgagtgcgcggactaaccgtcattgctcccagaggcttgcactctatcgcaggccttttccaataaggatggggcgttcggcggtgatgatgccggtcgtgcggggcatacggggagggtagatagaaaataacgacgctggtgttttggagaggggagggggactattaggggagggaaatacaggggcagggggtgagacgggtgacgttccggcggaacctcgcgcttgtcaaacaagcagccctgttaggttgctctagactagtgtacatacatacatatgtacatactgtatgtactgcacatactttaacttggtgcttccctgtgagccgccaggaacatcacaactgcaagcggaaaaggccccatatacggggcggcttgtcgggatggctccccccttcggaacgggtctgacttccgaggattttacctgcttcatttgggtattctgcgatggcctgttcaacccttcccctggccgaaccgtttcttggctcgatcctagtgtacactacactactcgtagactgcctgcccgacgatccgcgggaacgggccaggagtgtggagtggagacgggcggcggtgatgtcgtgtaattaaatatataagtgagagtgttttttgactgccccgggttctggtagttgaagggaagttcgatgctctctgctgtcgtcgctctcgtcgctctcgtcggcatcctccatccgtccgcctttgataacccgctccccgactcagtcaagacgacgcatacttggcaccatggtgactccctctctgaagaaggcagctctggctgccctgtccctcttcccgctcctctccctggctagcgaaaccgaaccttccggatatcagctggagaaggtggtcattctctcgcgtcacggtgtccgagcccccaccaagatgacacagacgatgcgcgatgtcactcctcatcagtggcctgagtggcccgtgaagctcggctacatcactcctcgtggagaacacctcatcagcctgatgggcggtttctatagggaacggttccagcagcagggattgcttcccaacgacacctgtccgacccccgacgccgtctacgtgtggaccgacgttaaccagcgtacccgcaagactggagaggctttcctcgccggtcttgcgcctcagtgtgatctggccatccaccaccagcagaacatcacgcaggctgacccgctgtttcacccggtcaaggccggtatctgttcgatgaacaagtctcagacctatgcggctgtcgagaagcaggctggcggccctattgagacgctaaaccagcgctaccaggccgaactggcattgatgtcctctgtgttggatttccccaagtccccatattgccagcagcataacatcggcaaactgtgcgacttttcacaggctatgcctagccgcctcaacatctccgatgacgggaatgaggtgcaactcgaaggcgccgtcggtctttcctccacgctcgccgagatcttcctactggaatacgctcagggtatgcctgtggtcgcctggggcaacattcacaacgagagccagtggaagagcctccttaacttgcacaacgcccatttcaacctgatgcacagaacgccctacattgccaagcaccagggaacccctttacttcaggctatcagcaacgctctcaacccaaatgcaactgagtcgaagctccccgatatctctcccgacaacaagatccttttcattgccggccacgacaccaacatcgcaaacatcggaggcatgttgggtatgaactggactctcccgggccagccagacaatactccgcccggcggtggactggttttcgaactctggcagaacccggataaccatcagcagtacgttgcggtgaagatgatctaccagaccatggaccagctgcgcaattccgagaagctggacttgaagagcaaccctgctgggatcgtccccattgagatcgaaggttgcgagaacatcggtaccgacaagctgtgccagctggatacttttcagaagcgtgttgcccaggtcattgagcccgcgtgccaaatctaatagcacgaacctctctgaaggaggttctgagacacgcgcgattcttctgtatatagttttatttttcactctggagtgcttcgctccaccagtacataaacctttttttcacgtaacaaaatggcttcttttcagaccatgtgaaccatcttgatgccttgacctcttcagttctcactttaacgtagttcgcgtttgtctgtatgtcccagatctacgtacaagacgagtgtgccagtcagccatcgaatgatgggtcgtcatggacaacggagcaatgccatctcgggactggggccaccgacctgccggcagggggaggagaaaaaaaagggggcggcgtcggcaagatggcaagaggtctgggagatagtcaaggacgaagggccaagcgggtcgtgtcgcttgcactctggtgaggctcgctagtgcttgcggtgggtgaggtggatcagagtagaggcccgatggagaatgggcgctgcggtgacaagcctaaccggtcgatagcggctagtaggctgggcggcgatgtcacgttatggttgccacggccgggcacggactgagggcatcgttcgaggacgtggagccgtgcgatggaagcgattaaggttgctgaagggtcggcgagggcaaccacacacctcattggaggagtctgggcgcggcagccaggttatcaggggtgacacggaaggcgtccgccgagtgcgactctgcgaagcccaggccgccctgcggaagaaccttggttcacgatgctctctgatgaccgcagaagagcgtatttgacgctccgcgcaaatgcgatccgaagtgccggtgcgaggctcctgtcgcctgctcgtctccgccctgactctcaacgcagcacgaacagcgctgccgtagatactccttcggagcctaccctggtgtacactagattcaagcaaaaggcgcacgagagcggagaaagccgcagcacaccccatcccttcccttcgcaacggatctcgcttctccccgcattttagtgtagtcccggtggcccacttaccccggcttctgcagtcttggtatttggtggtttctgatgtgcggggagagaggccttcagaaaaaaatgtgtctcttcgtggaactcgccgtgtgtgcgactttaacttgtatggaagttgaggtcgatttccccagatcttgaggctcaaaaactcccaaatttggcaccaaacgactgggacgcaaaaccgggtacctggtgccatcttcaatatacttgggaatgcagccgcggtgattgtcggtctgggcgcaacctcatcggcgttggcgggcgatcagcaggtgcaacggggggtgcaaaccgggaaatgagaagagcccagaccaacgaggaccgcggcatcgacctccgatcaccctaagttgggcctagtgccgccccaccaaggaacggaccagggtagtttgctcctcccgatcactggtagtcgtgccacagtgcagcgcattcaagggcaagccgctttcccaaatagagcaggaagggctggtttcaactttgaggtcaggacatccgtaacgttattccactttcggtgttctctttcttccgcagcttcttcggcagcgagttagttgcgcaactctcgtcacttttgcaatactttcatcgacgttccccaaatcggatacctcatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgctcgatttaaatctcgagaggcctgacgtcgggcccggtaccacgcgtcatatgactagt

SEQ ID NO: 6 - DNA, 9941 bp, synthetic construct
  source 1..9941: mol_type = other DNA
  misc_feature 1..9941: note = pMT2198
gaagaagggcgtaaataagaagctataatagcttaaaggtataattacctaacttagtaagcatactatattagccactatactataaagggctcctagaacgtaactacctatagcttagctaccactagctactataagggctagcgcccctcctagcttaatcgcctacctagtcgtatacctaccgcctacgactactagctagaaacgactacggctagaaacgctagcataggagagcccctcacgtaagagggctactagcgcgctctatagtaaccgtaccctagccagggacacctaggccagagctaggactctcgtacacctacagtaggagcgcccgtcagcactatccgtattcctagacaacctaggcctggctagtagtaagaaggactgacggataagcggatagatacgaccgtaggatagaggaacgattgtaaggacggtcataatagataaaagactatatttactaagttataacgggtcgtcatttttccttttgaaccctggtacctattatagcatttggctatctataggttagagctaactaacttactaatagggataataagctagggtcaatatatatttagatagcttctctaagtgcctctagtatttagaacaattaacgcttttattacctacttctgtagaccgtaacaattagtctaggagtgccgcttatagcgcgaatactgctgtggtatcatacctttacagcctctctaagctgtactgattatagccaccgagctatagcacaaatagctcgcttgacttggactttgatcaactacgatgcgaaaaagaaaagcgagaggaaaatgggaagaaaaaaatttttttaaaaaaaatgaaccatataatttaattagcaccgggattggctgttcgttgcctcttctagcttggttggggagagcggacggaatcccaaaatttccggcacgtatggtcgtttgtaattgggtctaccaaaagttgattagtagtgctcgcctgttgaagttaaggcgcctagaggcatataatattactaataatagtccttacactcttggggtccataaaccgtgtatccctgcgccgatgaatgtgctctccatttgagcaaatagaatagtcgacagcaccacgaattaacaggataaagtacggtatgatgggtcagttcagcaatgtgtgagtagtgtactgtggtaagaaggtaggcggtagagagcgggctctccagaccacagaaagagggctccgtcccacaaaggcccagacccacttccaggggtatttatctcaactacatgcaactgggacatacagacaaacgcgaactacgttaaagtgagaactgaagaggtcaaggcatcaagatggttcacatggtctgaaaagaagccattttgttacgtgaaaaaaaaggtttatgtactggtggagcgaagcactccagagtgaaaaataaaactatatacagaagaatcgcgcgtgtctcagaacctccttcagagaggttcgtcgtaataatgggcgctttctactctggacttgcctctttgtgctttacattgggggtcgagcctttccaatcagaagatgcacttaccaaagtagcgctaggctacacagcgctttacccccgaatcaggcgtggggtcactaccccgacgcccttttttttttttttttttgtattcacagagcggtttgcacaatctagatttgttcctcccgcctagtcggtcgccttgtacttggcccggtactcctccgtcctcctgatgtcctcctcggatgcgagccccttgataccctcgatgatgtcgtccaaggtgagaatcgcaaagatgggcagaccatactccttccggagctcaccgatcgcgctcggcatcggcttgctatcgtcgccatccggcgaaggcagcttctccatgcggtccagcgcgaccacgatgccggctacgataccgccctccttctcaatcttggcgatcgcctcacgcttcgccgtgccagccgtgatcacatcatccacgataagcaccctcttgcccttgagcggcgcccccacaatgttgccgccctcaccgtggtcctttgcctccttgcggtcgaacgagtagcacgtggtatggccgtacgtggccggatccagctggcccagccgaatggtggccgccgtcgccaacgggatccccttgtaggccgggccgaagacgatgtcgaactggatcgcgccgctccggtgcgcctcgatgatggcgtgcgcgtacgccgtcgcgagcgcctcgagcagatcggcccggtagaagtctcccgcgttgaagaagtagggcgagatgcgcttcgacttgagctcgaagctgccaaacttgaggatgttgccggcgatggccgcccggaggaagtcggctttgtaagaagcgagtggggccatgtctgtcggttcgtacgtgtcttacgggttcttgggctggaagtttatttgttaccggagggaactcctggtgaagattcgaaaaaaagaaatgagggagttttgtcaaacgtccccctatcgcagagctgaagagagcccctccatcgcgaatttcttggtggggttggattcatggtggggcccaagggtcgttaaacttttttgtatgtctacggagttacacagtactccgtacgtcgtaccccacttgcaatgcttctcgagctgggcttgggatattttgataacgcacaccgcctgccttatcttatcattttgtgctctccgcaaaaaattatttcgggcgacttggggacggcaaatcgacaccgccccgccaggtgctccctatctgacaactcccaaacacccccatcgccaaattcggacagcaccctgcgcgttgcctgccatggcctggaaggatatcgcgcctgtgcctacgtaagggttcaattcgtatatatgctgttcctttcctgaactaagctgatcattttcgtacagggcacaggagtttatcgacatcatcctttcgtaagtttgaaggctaggttatacgtagcggccgctcgatgccgcggtcctcgttggtctgggctcttctcatttcccggtttgcaccccccgttgcacctgctgatcgcccgccaacgccgatgaggttgcgcccagaccgacaatcaccgcggctgcattcccaagtatattgaagatggcaccaggtacccggttttgcgtcccagtcgtttggtgccaaatttgggagtttttgagcctcaagatctggggaaatcgacctcaacttccatacaagttaaagtcgcacacacggcgagttccacgaagagacacatttttttctgaaggcctctctccccgcacatcagaaaccaccaaataccaagactgcagaagccggggtaagtgggccaccgggactacactaaaatgcggggagaagcgagatccgttgcgaagggaagggatggggtgtgctgcggctttctccgctctcgtgcgccttttgcttgaatctagtgtacaccagggtaggctccgaaggagtatctacggcagcgctgttcgtgctgcgttgagagtcagggcggagacgagcaggcgacaggagcctcgcaccggcacttcggatcgcatttgcgcggagcgtcaaatacgctcttctgcggtcatcagagagcatcgtgaaccaaggttcttccgcagggcggcctgggcttcgcagagtcgcactcggcggacgccttccgtgtcacccctgataacctggctgccgcgcccagactcctccaatgaggtgtgtggttgccctcgccgacccttcagcaaccttaatcgcttccatcgcacggctccacgtcctcgaacgatgccctcagtccgtgcccggccgtggcaaccataacgtgacatcgccgcccagcctactagccgctatcgaccggttaggcttgtcaccgcagcgcccattctccatcgggcctctactctgatccacctcacccaccgcaagcactagcgagcctcaccagagtgcaagcgacacgacccgcttggcccttcgtccttgactatctcccagacctcttgccatcttgccgacgccgcccccttttttttctcctccccctgccggcaggtcggtggccccagtcccgagatggcattgctccgttgtccatgacgacccatcattcgatggctgactggcacactcgtcttgtttgagcatcgacggcccgcggcccgtctcccacggtacggaacctcgttgtacagtacctctcgtaatgatacccaacaccggggccgagcgctgggagggcggcgttcccgagaagccgggaaggcggctggccggctgacctttgtgacttggcgatggatgcggccatggagaatgtccgtccgaagcgacgcgacaattagcctggctaccatcgatataaattgggtgattcccagctcttgatgggcgtgtcttctgcctggcagccctcgtcttcagatcaagcaactgtgtgctgatcctcttccgccatggtgactccctctctgaagaaggcagctctggctgccctgtccctcttcccgctcctctccctggctagcgaaaccgaaccttccggatatcagctggagaaggtggtcattctctcgcgtcacggtgtccgagcccccaccaagatgacacagacgatgcgcgatgtcactcctcatcagtggcctgagtggcccgtgaagctcggctacatcactcctcgtggagaacacctcatcagcctgatgggcggtttctatagggaacggttccagcagcagggattgcttcccaacgacacctgtccgacccccgacgccgtctacgtgtggaccgacgttaaccagcgtacccgcaagactggagaggctttcctcgccggtcttgcgcctcagtgtgatctggccatccaccaccagcagaacatcacgcaggctgacccgctgtttcacccggtcaaggccggtatctgttcgatgaacaagtctcagacctatgcggctgtcgagaagcaggctggcggccctattgagacgctaaaccagcgctaccaggccgaactggcattgatgtcctctgtgttggatttccccaagtccccatattgccagcagcataacatcggcaaactgtgcgacttttcacaggctatgcctagccgcctcaacatctccgatgacgggaatgaggtgcaactcgaaggcgccgtcggtctttcctccacgctcgccgagatcttcctactggaatacgctcagggtatgcctgtggtcgcctggggcaacattcacaacgagagccagtggaagagcctccttaacttgcacaacgcccatttcaacctgatgcacagaacgccctacattgccaagcaccagggaacccctttacttcaggctatcagcaacgctctcaacccaaatgcaactgagtcgaagctccccgatatctctcccgacaacaagatccttttcattgccggccacgacaccaacatcgcaaacatcggaggcatgttgggtatgaactggactctcccgggccagccagacaatactccgcccggcggtggactggttttcgaactctggcagaacccggataaccatcagcagtacgttgcggtgaagatgatctaccagaccatggaccagctgcgcaattccgagaagctggacttgaagagcaaccctgctgggatcgtccccattgagatcgaaggttgcgagaacatcggtaccgacaagctgtgccagctggatacttttcagaagcgtgttgcccaggtcattgagcccgcgtgccaaatctaatagcacgaacctctctgaaggaggttctgagacacgcgcgattcttctgtatatagttttatttttcactctggagtgcttcgctccaccagtacataaacctttttttcacgtaacaaaatggcttcttttcagaccatgtgaaccatcttgatgccttgacctcttcagttctcactttaacgtagttcgcgtttgtctgtatgtcccagatctacgtacaagacgagtgtgccagtcagccatcgaatgatgggtcgtcatggacaacggagcaatgccatctcgggactggggccaccgacctgccggcagggggaggagaaaaaaaagggggcggcgtcggcaagatggcaagaggtctgggagatagtcaaggacgaagggccaagcgggtcgtgtcgcttgcactctggtgaggctcgctagtgcttgcggtgggtgaggtggatcagagtagaggcccgatggagaatgggcgctgcggtgacaagcctaaccggtcgatagcggctagtaggctgggcggcgatgtcacgttatggttgccacggccgggcacggactgagggcatcgttcgaggacgtggagccgtgcgatggaagcgattaaggttgctgaagggtcggcgagggcaaccacacacctcattggaggagtctgggcgcggcagccaggttatcaggggtgacacggaaggcgtccgccgagtgcgactctgcgaagcccaggccgccctgcggaagaaccttggttcacgatgctctctgatgaccgcagaagagcgtatttgacgctccgcgcaaatgcgatccgaagtgccggtgcgaggctcctgtcgcctgctcgtctccgccctgactctcaacgcagcacgaacagcgctgccgtagatactccttcggagcctaccctggtgtacactagattcaagcaaaaggcgcacgagagcggagaaagccgcagcacaccccatcccttcccttcgcaacggatctcgcttctccccgcattttagtgtagtcccggtggcccacttaccccggcttctgcagtcttggtatttggtggtttctgatgtgcggggagagaggccttcagaaaaaaatgtgtctcttcgtggaactcgccgtgtgtgcgactttaacttgtatggaagttgaggtcgatttccccagatcttgaggctcaaaaactcccaaatttggcaccaaacgactgggacgcaaaaccgggtacctggtgccatcttcaatatacttgggaatgcagccgcggtgattgtcggtctgggcgcaacctcatcggcgttggcgggcgatcagcaggtgcaacggggggtgcaaaccgggaaatgagaagagcccagaccaacgaggaccgcggcatcgacctccgatcaccctaagttgggcctagtgccgccccaccaaggaacggaccagggtagtttgctcctcccgatcactggtagtcgtgccacagtgcagcgcattcaagggcaagccgctttcccaaatagagcaggaagggctggtttcaactttgaggtcaggacatccgtaacgttattccactttcggtgttctctttcttccgcagcttcttcggcagcgagttagttgcgcaactctcgtcacttttgcaatactttcatcgacgttccccaaatcggatacctcatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgctcgatttaaatctcgagaggcctgacgtcgggcccggtaccacgcgtcatatgactagt

SEQ ID NO: 7 - DNA, 10607 bp, synthetic construct
  source 1..10607: mol_type = other DNA
  misc_feature 1..10607: note = pMT2443
gaagaagggcgtaaataagaagctataatagcttaaaggtataattacctaacttagtaagcatactatattagccactatactataaagggctcctagaacgtaactacctatagcttagctaccactagctactataagggctagcgcccctcctagcttaatcgcctacctagtcgtatacctaccgcctacgactactagctagaaacgactacggctagaaacgctagcataggagagcccctcacgtaagagggctactagcgcgctctatagtaaccgtaccctagccagggacacctaggccagagctaggactctcgtacacctacagtaggagcgcccgtcagcactatccgtattcctagacaacctaggcctggctagtagtaagaaggactgacggataagcggatagatacgaccgtaggatagaggaacgattgtaaggacggtcataatagataaaagactatatttactaagttataacgggtcgtcatttttccttttgaaccctggtacctattatagcatttggctatctataggttagagctaactaacttactaatagggataataagctagggtcaatatatatttagatagcttctctaagtgcctctagtatttagaacaattaacgcttttattacctacttctgtagaccgtaacaattagtctaggagtgccgcttatagcgcgaatactgctgtggtatcatacctttacagcctctctaagctgtactgattatagccaccgagctatagcacaaatagctcgcttgacttggactttgatcaactacgatgcgaaaaagaaaagcgagaggaaaatgggaagaaaaaaatttttttaaaaaaaatgaaccatataatttaattagcaccgggattggctgttcgttgcctcttctagcttggttggggagagcggacggaatcccaaaatttccggcacgtatggtcgtttgtaattgggtctaccaaaagttgattagtagtgctcgcctgttgaagttaaggcgcctagaggcatataatattactaataatagtccttacactcttggggtccataaaccgtgtatccctgcgccgatgaatgtgctctccatttgagcaaatagaatagtcgacagcaccacgaattaacaggataaagtacggtatgatgggtcagttcagcaatgtgtgagtagtgtactgtggtaagaaggtaggcggtagagagcgggctctccagaccacagaaagagggctccgtcccacaaaggcccagacccacttccaggggtatttatctcaactacatgcaactgggacatacagacaaacgcgaactacgttaaagtgagaactgaagaggtcaaggcatcaagatggttcacatggtctgaaaagaagccattttgttacgtgaaaaaaaaggtttatgtactggtggagcgaagcactccagagtgaaaaataaaactatatacagaagaatcgcgcgtgtctcagaacctccttcagagaggttcgtcgtaataatgggcgctttctactctggacttgcctctttgtgctttacattgggggtcgagcctttccaatcagaagatgcacttaccaaagtagcgctaggctacacagcgctttacccccgaatcaggcgtggggtcactaccccgacgcccttttttttttttttttttgtattcacagagcggtttgcacaatctagatttgttcctcccgcctagtcggtcgccttgtacttggcccggtactcctccgtcctcctgatgtcctcctcggatgcgagccccttgataccctcgatgatgtcgtccaaggtgagaatcgcaaagatgggcagaccatactccttccggagctcaccgatcgcgctcggcatcggcttgctatcgtcgccatccggcgaaggcagcttctccatgcggtccagcgcgaccacgatgccggctacgataccgccctccttctcaatcttggcgatcgcctcacgcttcgccgtgccagccgtgatcacatcatccacgataagcaccctcttgcccttgagcggcgcccccacaatgttgccgccctcaccgtggtcctttgcctccttgcggtcgaacgagtagcacgtggtatggccgtacgtggccggatccagctggcccagccgaatggtggccgccgtcgccaacgggatccccttgtaggccgggccgaagacgatgtcgaactggatcgcgccgctccggtgcgcctcgatgatggcgtgcgcgtacgccgtcgcgagcgcctcgagcagatcggcccggtagaagtctcccgcgttgaagaagtagggcgagatgcgcttcgacttgagctcgaagctgccaaacttgaggatgttgccggcgatggccgcccggaggaagtcggctttgtaagaagcgagtggggccatgtctgtcggttcgtacgtgtcttacgggttcttgggctggaagtttatttgttaccggagggaactcctggtgaagattcgaaaaaaagaaatgagggagttttgtcaaacgtccccctatcgcagagctgaagagagcccctccatcgcgaatttcttggtggggttggattcatggtggggcccaagggtcgttaaacttttttgtatgtctacggagttacacagtactccgtacgtcgtaccccacttgcaatgcttctcgagctgggcttgggatattttgataacgcacaccgcctgccttatcttatcattttgtgctctccgcaaaaaattatttcgggcgacttggggacggcaaatcgacaccgccccgccaggtgctccctatctgacaactcccaaacacccccatcgccaaattcggacagcaccctgcgcgttgcctgccatggcctggaaggatatcgcgcctgtgcctacgtaagggttcaattcgtatatatgctgttcctttcctgaactaagctgatcattttcgtacagggcacaggagtttatcgacatcatcctttcgtaagtttgaaggctaggttatacgtagcggccgctcgaccaatagcagagtgcggagaagtctgagagagactctggcagcggagtcccttgcactttaattacttgatactccatataccgtttcggccactcagattctaggggtagggcgggagcagaggcgaaaattgggttgtagaatatgaggagctagggttgttaaactcaaagaacttcttgctcttgttcttagtcttctctcctgggaaaagggggtttttccgaaagcggcgctatacgaagccagaggctactttccttgctttggatggcccttgtccaccgttcttgtttcccgtttgtcaattgcgacgttgccggcaacctaggtcctaataattaggtagatatttcggtagaggtagtttaattatgcttcagtagagaaatcgttgtctccacgtctcgcaaccttgcgaaacttcgccacattgaagatagcattgtctgagttgattttaaccctttccagagacgatataatagtgcaagtttctttgatcggaatcatcgacattcggattttcccttaattatatgaagtattcggcccacggaaccgggccccgagcaggttgaaccgcgcaaaacctcaaccgagtcacctcgcgtccatgtttgtcatggaatcaggctccgaatcccgtcagatcagtcagttctggtggctatggacgcgggagttacggccagtcgtcccgttgttctggggggttgatcaacaggaggaagagatctgagatcgaactacacccattgatttatcgacgcataatcaagtttaataaaaaccaaacagcgtgtttggtgctaccaccgaatgcgagatccgggctagcccgcggaaggatgatggccacagatctagcgtcatgtatgattattacctatgcatctatcttcgtatctgcctcgggttggcaacacctgaccgagagacgactcgacaacctgacacttggcaaaagacatttcggttgacagcgggagaactccagcgaggaagtcgcccagagatgcggatgagaagacaacgccgagacgtgccggcgttggctctccacgaatcggagccgactcttccgtttggccaatctccgggataaatcccagcggcgggtcacgtcacgtttcatggggaggcgcggacagccatcccagccaggccatggaagagaacaattcttgggggtagcgaccgagccaaaaggggggggggggaagcgggaggggaagaagtggtattagagcacgcaccggaaaacgcatttgggcccttgccaacaaacaccacaccccgcgtcctgggagcaagacatccaggatgcaacccagtaggggatgccaagaagcatctacggcaccatctgccggcgcctcgcctgttagagtcccggcacccgccaatggggccgtgctgggccctgcccggcaatgctggcgcagcggcatcaacaacattgctcggggaggggcccgattttattgattagcaaaaaaacaattaaattacccttccattccagcagagcttctcctccacgcggcggcgggaccgcttgtggacggcggtacactacaaccgcggggctccagtctccgtgctgggcgtgcagatcacgacccggaagagaaatgatcgcggtctgacgccgggtacggagtactgagccgccaaccacagccgatggaccgtgatatctcaatgcgttcaagcaacacagcaacaccctggacgagtctctcctcccctaccaccccctccccccctgccctggccgcgaacggggcgcgtaccccagatttctactccgtactgacaccccaatctattcccgctggcgtcgcccagtctggggcggtccggccaagactctcggtgcacgataccgcgacgaaatcggattaaccgttggctgatcaattccaagtcaagggagaagtggtatggaaagtcggctcagttttccactgcccccgacaggcaggttccggatctggacagcagtcttccgaatctttggcagagactcatgataatataaaaaggcaaatgaggcggcgccttggacaggtccattctcccaccgctcaaccagcctccaattcctcagaagtctgttgctctctcgcagtcgcagtcaagccatggtgactccctctctgaagaaggcagctctggctgccctgtccctcttcccgctcctctccctggctagcgaaaccgaaccttccggatatcagctggagaaggtggtcattctctcgcgtcacggtgtccgagcccccaccaagatgacacagacgatgcgcgatgtcactcctcatcagtggcctgagtggcccgtgaagctcggctacatcactcctcgtggagaacacctcatcagcctgatgggcggtttctatagggaacggttccagcagcagggattgcttcccaacgacacctgtccgacccccgacgccgtctacgtgtggaccgacgttaaccagcgtacccgcaagactggagaggctttcctcgccggtcttgcgcctcagtgtgatctggccatccaccaccagcagaacatcacgcaggctgacccgctgtttcacccggtcaaggccggtatctgttcgatgaacaagtctcagacctatgcggctgtcgagaagcaggctggcggccctattgagacgctaaaccagcgctaccaggccgaactggcattgatgtcctctgtgttggatttccccaagtccccatattgccagcagcataacatcggcaaactgtgcgacttttcacaggctatgcctagccgcctcaacatctccgatgacgggaatgaggtgcaactcgaaggcgccgtcggtctttcctccacgctcgccgagatcttcctactggaatacgctcagggtatgcctgtggtcgcctggggcaacattcacaacgagagccagtggaagagcctccttaacttgcacaacgcccatttcaacctgatgcacagaacgccctacattgccaagcaccagggaacccctttacttcaggctatcagcaacgctctcaacccaaatgcaactgagtcgaagctccccgatatctctcccgacaacaagatccttttcattgccggccacgacaccaacatcgcaaacatcggaggcatgttgggtatgaactggactctcccgggccagccagacaatactccgcccggcggtggactggttttcgaactctggcagaacccggataaccatcagcagtacgttgcggtgaagatgatctaccagaccatggaccagctgcgcaattccgagaagctggacttgaagagcaaccctgctgggatcgtccccattgagatcgaaggttgcgagaacatcggtaccgacaagctgtgccagctggatacttttcagaagcgtgttgcccaggtcattgagcccgcgtgccaaatctaatagcacgaacctctctgaaggaggttctgagacacgcgcgattcttctgtatatagttttatttttcactctggagtgcttcgctccaccagtacataaacctttttttcacgtaacaaaatggcttcttttcagaccatgtgaaccatcttgatgccttgacctcttcagttctcactttaacgtagttcgcgtttgtctgtatgtcccagatctacgtacaagacgagtgtgccagtcagccatcgaatgatgggtcgtcatggacaacggagcaatgccatctcgggactggggccaccgacctgccggcagggggaggagaaaaaaaagggggcggcgtcggcaagatggcaagaggtctgggagatagtcaaggacgaagggccaagcgggtcgtgtcgcttgcactctggtgaggctcgctagtgcttgcggtgggtgaggtggatcagagtagaggcccgatggagaatgggcgctgcggtgacaagcctaaccggtcgatagcggctagtaggctgggcggcgatgtcacgttatggttgccacggccgggcacggactgagggcatcgttcgaggacgtggagccgtgcgatggaagcgattaaggttgctgaagggtcggcgagggcaaccacacacctcattggaggagtctgggcgcggcagccaggttatcaggggtgacacggaaggcgtccgccgagtgcgactctgcgaagcccaggccgccctgcggaagaaccttggttcacgatgctctctgatgaccgcagaagagcgtatttgacgctccgcgcaaatgcgatccgaagtgccggtgcgaggctcctgtcgcctgctcgtctccgccctgactctcaacgcagcacgaacagcgctgccgtagatactccttcggagcctaccctggtgtacactagattcaagcaaaaggcgcacgagagcggagaaagccgcagcacaccccatcccttcccttcgcaacggatctcgcttctccccgcattttagtgtagtcccggtggcccacttaccccggcttctgcagtcttggtatttggtggtttctgatgtgcggggagagaggccttcagaaaaaaatgtgtctcttcgtggaactcgccgtgtgtgcgactttaacttgtatggaagttgaggtcgatttccccagatcttgaggctcaaaaactcccaaatttggcaccaaacgactgggacgcaaaaccgggtacctggtgccatcttcaatatacttgggaatgcagccgcggtgattgtcggtctgggcgcaacctcatcggcgttggcgggcgatcagcaggtgcaacggggggtgcaaaccgggaaatgagaagagcccagaccaacgaggaccgcggcatcgacctccgatcaccctaagttgggcctagtgccgccccaccaaggaacggaccagggtagtttgctcctcccgatcactggtagtcgtgccacagtgcagcgcattcaagggcaagccgctttcccaaatagagcaggaagggctggtttcaactttgaggtcaggacatccgtaacgttattccactttcggtgttctctttcttccgcagcttcttcggcagcgagttagttgcgcaactctcgtcacttttgcaatactttcatcgacgttccccaaatcggatacctcatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgctcgatttaaatctcgagaggcctgacgtcgggcccggtaccacgcgtcatatgactagt

SEQ ID NO: 8 - DNA, 10088 bp, synthetic construct
  source 1..10088: mol_type = other DNA
  misc_feature 1..10088: note = pMT2193
gaagaagggcgtaaataagaagctataatagcttaaaggtataattacctaacttagtaagcatactatattagccactatactataaagggctcctagaacgtaactacctatagcttagctaccactagctactataagggctagcgcccctcctagcttaatcgcctacctagtcgtatacctaccgcctacgactactagctagaaacgactacggctagaaacgctagcataggagagcccctcacgtaagagggctactagcgcgctctatagtaaccgtaccctagccagggacacctaggccagagctaggactctcgtacacctacagtaggagcgcccgtcagcactatccgtattcctagacaacctaggcctggctagtagtaagaaggactgacggataagcggatagatacgaccgtaggatagaggaacgattgtaaggacggtcataatagataaaagactatatttactaagttataacgggtcgtcatttttccttttgaaccctggtacctattatagcatttggctatctataggttagagctaactaacttactaatagggataataagctagggtcaatatatatttagatagcttctctaagtgcctctagtatttagaacaattaacgcttttattacctacttctgtagaccgtaacaattagtctaggagtgccgcttatagcgcgaatactgctgtggtatcatacctttacagcctctctaagctgtactgattatagccaccgagctatagcacaaatagctcgcttgacttggactttgatcaactacgatgcgaaaaagaaaagcgagaggaaaatgggaagaaaaaaatttttttaaaaaaaatgaaccatataatttaattagcaccgggattggctgttcgttgcctcttctagcttggttggggagagcggacggaatcccaaaatttccggcacgtatggtcgtttgtaattgggtctaccaaaagttgattagtagtgctcgcctgttgaagttaaggcgcctagaggcatataatattactaataatagtccttacactcttggggtccataaaccgtgtatccctgcgccgatgaatgtgctctccatttgagcaaatagaatagtcgacagcaccacgaattaacaggataaagtacggtatgatgggtcagttcagcaatgtgtgagtagtgtactgtggtaagaaggtaggcggtagagagcgggctctccagaccacagaaagagggctccgtcccacaaaggcccagacccacttccaggggtatttatctcaactacatgcaactgggacatacagacaaacgcgaactacgttaaagtgagaactgaagaggtcaaggcatcaagatggttcacatggtctgaaaagaagccattttgttacgtgaaaaaaaaggtttatgtactggtggagcgaagcactccagagtgaaaaataaaactatatacagaagaatcgcgcgtgtctcagaacctccttcagagaggttcgtcgtaataatgggcgctttctactctggacttgcctctttgtgctttacattgggggtcgagcctttccaatcagaagatgcacttaccaaagtagcgctaggctacacagcgctttacccccgaatcaggcgtggggtcactaccccgacgcccttttttttttttttttttgtattcacagagcggtttgcacaatctagatttgttcctcccgcctagtcggtcgccttgtacttggcccggtactcctccgtcctcctgatgtcctcctcggatgcgagccccttgataccctcgatgatgtcgtccaaggtgagaatcgcaaagatgggcagaccatactccttccggagctcaccgatcgcgctcggcatcggcttgctatcgtcgccatccggcgaaggcagcttctccatgcggtccagcgcgaccacgatgccggctacgataccgccctccttctcaatcttggcgatcgcctcacgcttcgccgtgccagccgtgatcacatcatccacgataagcaccctcttgcccttgagcggcgcccccacaatgttgccgccctcaccgtggtcctttgcctccttgcggtcgaacgagtagcacgtggtatggccgtacgtggccggatccagctggcccagccgaatggtggccgccgtcgccaacgggatccccttgtaggccgggccgaagacgatgtcgaactggatcgcgccgctccggtgcgcctcgatgatggcgtgcgcgtacgccgtcgcgagcgcctcgagcagatcggcccggtagaagtctcccgcgttgaagaagtagggcgagatgcgcttcgacttgagctcgaagctgccaaacttgaggatgttgccggcgatggccgcccggaggaagtcggctttgtaagaagcgagtggggccatgtctgtcggttcgtacgtgtcttacgggttcttgggctggaagtttatttgttaccggagggaactcctggtgaagattcgaaaaaaagaaatgagggagttttgtcaaacgtccccctatcgcagagctgaagagagcccctccatcgcgaatttcttggtggggttggattcatggtggggcccaagggtcgttaaacttttttgtatgtctacggagttacacagtactccgtacgtcgtaccccacttgcaatgcttctcgagctgggcttgggatattttgataacgcacaccgcctgccttatcttatcattttgtgctctccgcaaaaaattatttcgggcgacttggggacggcaaatcgacaccgccccgccaggtgctccctatctgacaactcccaaacacccccatcgccaaattcggacagcaccctgcgcgttgcctgccatggcctggaaggatatcgcgcctgtgcctacgtaagggttcaattcgtatatatgctgttcctttcctgaactaagctgatcattttcgtacagggcacaggagtttatcgacatcatcctttcgtaagtttgaaggctaggttatacgtagcggccgctcgacattcggattttcccttaattatatgaagtattcggcccacggaaccgggccccgagcaggttgaaccgcgcaaaacctcaaccgagtcacctcgcgtccatgtttgtcatggaatcaggctccgaatcccgtcagatcagtcagttctggtggctatggacgcgggagttacggccagtcgtcccgttgttctggggggttgatcaacaggaggaagagatctgagatcgaactacacccattgatttatcgacgcataatcaagtttaataaaaaccaaacagcgtgtttggtgctaccaccgaatgcgagatccgggctagcccgcggaaggatgatggccacagatctagcgtcatgtatgattattacctatgcatctatcttcgtatctgcctcgggttggcaacacctgaccgagagacgactcgacaacctgacacttggcaaaagacatttcggttgacagcgggagaactccagcgaggaagtcgcccagagatgcggatgagaagacaacgccgagacgtgccggcgttggctctccacgaatcggagccgactcttccgtttggccaatctccgggataaatcccagcggcgggtcacgtcacgtttcatggggaggcgcggacagccatcccagccaggccatggaagagaacaattcttgggggtagcgaccgagccaaaaggggggggggggaagcgggaggggaagaagtggtattagagcacgcaccggaaaacgcatttgggcccttgccaacaaacaccacaccccgcgtcctgggagcaagacatccaggatgcaacccagtaggggatgccaagaagcatctacggcaccatctgccggcgcctcgcctgttagagtcccggcacccgccaatggggccgtgctgggccctgcccggcaatgctggcgcagcggcatcaacaacattgctcggggaggggcccgattttattgattagcaaaaaaacaattaaattacccttccattccagcagagcttctcctccacgcggcggcgggaccgcttgtggacggcggtacactacaaccgcggggctccagtctccgtgctgggcgtgcagatcacgacccggaagagaaatgatcgcggtctgacgccgggtacggagtactgagccgccaaccacagccgatggaccgtgatatctcaatgcgttcaagcaacacagcaacaccctggacgagtctctcctcccctaccaccccctccccccctgccctggccgcgaacggggcgcgtaccccagatttctactccgtactgacaccccaatctattcccgctggcgtcgcccagtctggggcggtccggccaagactctcggtgcacgataccgcgacgaaatcggattaaccgttggctgatcaattccaagtcaagggagaagtggtatggaaagtcggctcagttttccactgcccccgacaggcaggttccggatctggacagcagtcttccgaatctttggcagagactcatgataatataaaaaggcaaatgaggcggcgccttggacaggtccattctcccaccgctcaaccagcctccaattcctcagaagtctgttgctctctcgcagtcgcagtcaccatggtgactccctctctgaagaaggcagctctggctgccctgtccctcttcccgctcctctccctggctagcgaaaccgaaccttccggatatcagctggagaaggtggtcattctctcgcgtcacggtgtccgagcccccaccaagatgacacagacgatgcgcgatgtcactcctcatcagtggcctgagtggcccgtgaagctcggctacatcactcctcgtggagaacacctcatcagcctgatgggcggtttctatagggaacggttccagcagcagggattgcttcccaacgacacctgtccgacccccgacgccgtctacgtgtggaccgacgttaaccagcgtacccgcaagactggagaggctttcctcgccggtcttgcgcctcagtgtgatctggccatccaccaccagcagaacatcacgcaggctgacccgctgtttcacccggtcaaggccggtatctgttcgatgaacaagtctcagacctatgcggctgtcgagaagcaggctggcggccctattgagacgctaaaccagcgctaccaggccgaactggcattgatgtcctctgtgttggatttccccaagtccccatattgccagcagcataacatcggcaaactgtgcgacttttcacaggctatgcctagccgcctcaacatctccgatgacgggaatgaggtgcaactcgaaggcgccgtcggtctttcctccacgctcgccgagatcttcctactggaatacgctcagggtatgcctgtggtcgcctggggcaacattcacaacgagagccagtggaagagcctccttaacttgcacaacgcccatttcaacctgatgcacagaacgccctacattgccaagcaccagggaacccctttacttcaggctatcagcaacgctctcaacccaaatgcaactgagtcgaagctccccgatatctctcccgacaacaagatccttttcattgccggccacgacaccaacatcgcaaacatcggaggcatgttgggtatgaactggactctcccgggccagccagacaatactccgcccggcggtggactggttttcgaactctggcagaacccggataaccatcagcagtacgttgcggtgaagatgatctaccagaccatggaccagctgcgcaattccgagaagctggacttgaagagcaaccctgctgggatcgtccccattgagatcgaaggttgcgagaacatcggtaccgacaagctgtgccagctggatacttttcagaagcgtgttgcccaggtcattgagcccgcgtgccaaatctaatagcacgaacctctctgaaggaggttctgagacacgcgcgattcttctgtatatagttttatttttcactctggagtgcttcgctccaccagtacataaacctttttttcacgtaacaaaatggcttcttttcagaccatgtgaaccatcttgatgccttgacctcttcagttctcactttaacgtagttcgcgtttgtctgtatgtcccagatctacgtacaagacgagtgtgccagtcagccatcgaatgatgggtcgtcatggacaacggagcaatgccatctcgggactggggccaccgacctgccggcagggggaggagaaaaaaaagggggcggcgtcggcaagatggcaagaggtctgggagatagtcaaggacgaagggccaagcgggtcgtgtcgcttgcactctggtgaggctcgctagtgcttgcggtgggtgaggtggatcagagtagaggcccgatggagaatgggcgctgcggtgacaagcctaaccggtcgatagcggctagtaggctgggcggcgatgtcacgttatggttgccacggccgggcacggactgagggcatcgttcgaggacgtggagccgtgcgatggaagcgattaaggttgctgaagggtcggcgagggcaaccacacacctcattggaggagtctgggcgcggcagccaggttatcaggggtgacacggaaggcgtccgccgagtgcgactctgcgaagcccaggccgccctgcggaagaaccttggttcacgatgctctctgatgaccgcagaagagcgtatttgacgctccgcgcaaatgcgatccgaagtgccggtgcgaggctcctgtcgcctgctcgtctccgccctgactctcaacgcagcacgaacagcgctgccgtagatactccttcggagcctaccctggtgtacactagattcaagcaaaaggcgcacgagagcggagaaagccgcagcacaccccatcccttcccttcgcaacggatctcgcttctccccgcattttagtgtagtcccggtggcccacttaccccggcttctgcagtcttggtatttggtggtttctgatgtgcggggagagaggccttcagaaaaaaatgtgtctcttcgtggaactcgccgtgtgtgcgactttaacttgtatggaagttgaggtcgatttccccagatcttgaggctcaaaaactcccaaatttggcaccaaacgactgggacgcaaaaccgggtacctggtgccatcttcaatatacttgggaatgcagccgcggtgattgtcggtctgggcgcaacctcatcggcgttggcgggcgatcagcaggtgcaacggggggtgcaaaccgggaaatgagaagagcccagaccaacgaggaccgcggcatcgacctccgatcaccctaagttgggcctagtgccgccccaccaaggaacggaccagggtagtttgctcctcccgatcactggtagtcgtgccacagtgcagcgcattcaagggcaagccgctttcccaaatagagcaggaagggctggtttcaactttgaggtcaggacatccgtaacgttattccactttcggtgttctctttcttccgcagcttcttcggcagcgagttagttgcgcaactctcgtcacttttgcaatactttcatcgacgttccccaaatcggatacctcatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgctcgatttaaatctcgagaggcctgacgtcgggcccggtaccacgcgtcatatgactagt

SEQ ID NO: 9 - DNA, 9755 bp, synthetic construct
  source 1..9755: mol_type = other DNA
  misc_feature 1..9755: note = pMT2650
gaagaagggcgtaaataagaagctataatagcttaaaggtataattacctaacttagtaagcatactatattagccactatactataaagggctcctagaacgtaactacctatagcttagctaccactagctactataagggctagcgcccctcctagcttaatcgcctacctagtcgtatacctaccgcctacgactactagctagaaacgactacggctagaaacgctagcataggagagcccctcacgtaagagggctactagcgcgctctatagtaaccgtaccctagccagggacacctaggccagagctaggactctcgtacacctacagtaggagcgcccgtcagcactatccgtattcctagacaacctaggcctggctagtagtaagaaggactgacggataagcggatagatacgaccgtaggatagaggaacgattgtaaggacggtcataatagataaaagactatatttactaagttataacgggtcgtcatttttccttttgaaccctggtacctattatagcatttggctatctataggttagagctaactaacttactaatagggataataagctagggtcaatatatatttagatagcttctctaagtgcctctagtatttagaacaattaacgcttttattacctacttctgtagaccgtaacaattagtctaggagtgccgcttatagcgcgaatactgctgtggtatcatacctttacagcctctctaagctgtactgattatagccaccgagctatagcacaaatagctcgcttgacttggactttgatcaactacgatgcgaaaaagaaaagcgagaggaaaatgggaagaaaaaaatttttttaaaaaaaatgaaccatataatttaattagcaccgggattggctgttcgttgcctcttctagcttggttggggagagcggacggaatcccaaaatttccggcacgtatggtcgtttgtaattgggtctaccaaaagttgattagtagtgctcgcctgttgaagttaaggcgcctagaggcatataatattactaataatagtccttacactcttggggtccataaaccgtgtatccctgcgccgatgaatgtgctctccatttgagcaaatagaatagtcgacagcaccacgaattaacaggataaagtacggtatgatgggtcagttcagcaatgtgtgagtagtgtactgtggtaagaaggtaggcggtagagagcgggctctccagaccacagaaagagggctccgtcccacaaaggcccagacccacttccaggggtatttatctcaactacatgcaactgggacatacagacaaacgcgaactacgttaaagtgagaactgaagaggtcaaggcatcaagatggttcacatggtctgaaaagaagccattttgttacgtgaaaaaaaaggtttatgtactggtggagcgaagcactccagagtgaaaaataaaactatatacagaagaatcgcgcgtgtctcagaacctccttcagagaggttcgtcgtaataatgggcgctttctactctggacttgcctctttgtgctttacattgggggtcgagcctttccaatcagaagatgcacttaccaaagtagcgctaggctacacagcgctttacccccgaatcaggcgtggggtcactaccccgacgcccttttttttttttttttttgtattcacagagcggtttgcacaatctagatttgttcctcccgcctagtcggtcgccttgtacttggcccggtactcctccgtcctcctgatgtcctcctcggatgcgagccccttgataccctcgatgatgtcgtccaaggtgagaatcgcaaagatgggcagaccatactccttccggagctcaccgatcgcgctcggcatcggcttgctatcgtcgccatccggcgaaggcagcttctccatgcggtccagcgcgaccacgatgccggctacgataccgccctccttctcaatcttggcgatcgcctcacgcttcgccgtgccagccgtgatcacatcatccacgataagcaccctcttgcccttgagcggcgcccccacaatgttgccgccctcaccgtggtcctttgcctccttgcggtcgaacgagtagcacgtggtatggccgtacgtggccggatccagctggcccagccgaatggtggccgccgtcgccaacgggatccccttgtaggccgggccgaagacgatgtcgaactggatcgcgccgctccggtgcgcctcgatgatggcgtgcgcgtacgccgtcgcgagcgcctcgagcagatcggcccggtagaagtctcccgcgttgaagaagtagggcgagatgcgcttcgacttgagctcgaagctgccaaacttgaggatgttgccggcgatggccgcccggaggaagtcggctttgtaagaagcgagtggggccatgtctgtcggttcgtacgtgtcttacgggttcttgggctggaagtttatttgttaccggagggaactcctggtgaagattcgaaaaaaagaaatgagggagttttgtcaaacgtccccctatcgcagagctgaagagagcccctccatcgcgaatttcttggtggggttggattcatggtggggcccaagggtcgttaaacttttttgtatgtctacggagttacacagtactccgtacgtcgtaccccacttgcaatgcttctcgagctgggcttgggatattttgataacgcacaccgcctgccttatcttatcattttgtgctctccgcaaaaaattatttcgggcgacttggggacggcaaatcgacaccgccccgccaggtgctccctatctgacaactcccaaacacccccatcgccaaattcggacagcaccctgcgcgttgcctgccatggcctggaaggatatcgcgcctgtgcctacgtaagggttcaattcgtatatatgctgttcctttcctgaactaagctgatcattttcgtacagggcacaggagtttatcgacatcatcctttcgtaagtttgaaggctaggttatacgtagcggccgctcgagtctcttgttgtgaaccttgctgttgcatgggcaggaccggtgcatcatgaacaggacggtgcgctgtgttttttttttctcggggtcttgattgtttgttgaatctcccttttcgaggatacgagctctctcggggacgaatagatgaaggcaatctgacagatttgctctcaaaaaaagactgatatctcttccaccatgcactgtatgtacattacatacattatccccctccactggattcgcacaacggaaagcaatggcgcgctgattcaagaaccatcagggctgtcattggcttgttttgtgccgtggccgcggtgacgcccactatgactctctgggcaggcggcaactgggtgccagatatattaatccggggcatagcgcatatcttccttgatttgtagagtactagtacactaacccccttctccacatggggccactgttcggtagatctgcccgaagtgcaagtgcggggggggccaaactaggtaatatcctcccgctctcccgagtgcgcggactaaccgtcattgctcccagaggcttgcactctatcgcaggccttttccaataaggatggggcgttcggcggtgatgatgccggtcgtgcggggcatacggggagggtagatagaaaataacgacgctggtgttttggagaggggagggggactattaggggagggaaatacaggggcagggggtgagacgggtgacgttccggcggaacctcgcgcttgtcaaacaagcagccctgttaggttgctctagactagtgtacatacatacatatgtacatactgtatgtactgcacatactttaacttggtgcttccctgtgagccgccaggaacatcacaactgcaagcggaaaaggccccatatacggggcggcttgtcgggatggctccccccttcggaacgggtctgacttccgaggattttacctgcttcatttgggtattctgcgatggcctgttcaacccttcccctggccgaaccgtttcttggctcgatcctagtgtacactacactactcgtagactgcctgcccgacgatccgcgggaacgggccaggagtgtggagtggagacgggcggcggtgatgtcgtgtaattaaatatataagtgagagtgttttttgactgccccgggttctggtagttgaagggaagttcgatgctctctgctgtcgtcgctctcgtcgctctcgtcggcatcctccatccgtccgcctttgataacccgctccccgactcagtcaagacgacgcatacttggcaccatggtgactccctctctgaagaaggcagctctggctgccctgtccctcttcccgctcctctccctggctagcgaaaccgaaccttccggatatcagctggagaaggtggtcattctctcgcgtcacggtgtccgagcccccaccaagatgacacagacgatgcgcgatgtcactcctcatcagtggcctgagtggcccgtgaagctcggctacatcactcctcgtggagaacacctcatcagcctgatgggcggtttctatagggaacggttccagcagcagggattgcttcccaacgacacctgtccgacccccgacgccgtctacgtgtggaccgacgttaaccagcgtacccgcaagactggagaggctttcctcgccggtcttgcgcctcagtgtgatctggccatccaccaccagcagaacatcacgcaggctgacccgctgtttcacccggtcaaggccggtatctgttcgatgaacaagtctcagacctatgcggctgtcgagaagcaggctggcggccctattgagacgctaaaccagcgctaccaggccgaactggcattgatgtcctctgtgttggatttccccaagtccccatattgccagcagcataacatcggcaaactgtgcgacttttcacaggctatgcctagccgcctcaacatctccgatgacgggaatgaggtgcaactcgaaggcgccgtcggtctttcctccacgctcgccgagatcttcctactggaatacgctcagggtatgcctgtggtcgcctggggcaacattcacaacgagagccagtggaagagcctccttaacttgcacaacgcccatttcaacctgatgcacagaacgccctacattgccaagcaccagggaacccctttacttcaggctatcagcaacgctctcaacccaaatgcaactgagtcgaagctccccgatatctctcccgacaacaagatccttttcattgccggccacgacaccaacatcgcaaacatcggaggcatgttgggtatgaactggactctcccgggccagccagacaatactccgcccggcggtggactggttttcgaactctggcagaacccggataaccatcagcagtacgttgcggtgaagatgatctaccagaccatggaccagctgcgcaattccgagaagctggacttgaagagcaaccctgctgggatcgtccccattgagatcgaaggttgcgagaacatcggtaccgacaagctgtgccagctggatacttttcagaagcgtgttgcccaggtcattgagcccgcgtgccaaatctaatagcacgaacctctctgaaggaggttctgagacacgcgcgattcttctgtatatagttttatttttcactctggagtgcttcgctccaccagtacataaacctttttttcacgtaacaaaatggcttcttttcagaccatgtgaaccatcttgatgccttgacctcttcagttctcactttaacgtagttcgcgtttgtctgtatgtcccagatctacgtacaagacgagtgtgccagtcagccatcgaatgatgggtcgtcatggacaacggagcaatgccatctcgggactggggccaccgacctgccggcagggggaggagaaaaaaaagggggcggcgtcggcaagatggcaagaggtctgggagatagtcaaggacgaagggccaagcgggtcgtgtcgcttgcactctggtgaggctcgctagtgcttgcggtgggtgaggtggatcagagtagaggcccgatggagaatgggcgctgcggtgacaagcctaaccggtcgatagcggctagtaggctgggcggcgatgtcacgttatggttgccacggccgggcacggactgagggcatcgttcgaggacgtggagccgtgcgatggaagcgattaaggttgctgaagggtcggcgagggcaaccacacacctcattggaggagtctgggcgcggcagccaggttatcaggggtgacacggaaggcgtccgccgagtgcgactctgcgaagcccaggccgccctgcggaagaaccttggttcacgatgctctctgatgaccgcagaagagcgtatttgacgctccgcgcaaatgcgatccgaagtgccggtgcgaggctcctgtcgcctgctcgtctccgccctgactctcaacgcagcacgaacagcgctgccgtagatactccttcggagcctaccctggtgtacactagattcaagcaaaaggcgcacgagagcggagaaagccgcagcacaccccatcccttcccttcgcaacggatctcgcttctccccgcattttagtgtagtcccggtggcccacttaccccggcttctgcagtcttggtatttggtggtttctgatgtgcggggagagaggccttcagaaaaaaatgtgtctcttcgtggaactcgccgtgtgtgcgactttaacttgtatggaagttgaggtcgatttccccagatcttgaggctcaaaaactcccaaatttggcaccaaacgactgggacgcaaaaccgggtacctggtgccatcttcaatatacttgggaatgcagccgcggtgattgtcggtctgggcgcaacctcatcggcgttggcgggcgatcagcaggtgcaacggggggtgcaaaccgggaaatgagaagagcccagaccaacgaggaccgcggcatcgacctccgatcaccctaagttgggcctagtgccgccccaccaaggaacggaccagggtagtttgctcctcccgatcactggtagtcgtgccacagtgcagcgcattcaagggcaagccgctttcccaaatagagcaggaagggctggtttcaactttgaggtcaggacatccgtaacgttattccactttcggtgttctctttcttccgcagcttcttcggcagcgagttagttgcgcaactctcgtcacttttgcaatactttcatcgacgttccccaaatcggatacctcatcgatgctcttctgcgttaattaacaattgggatcctctagacccgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgctcgatttaaatctcgagaggcctgacgtcgggcccggtaccacgcgtcatatgactagt

SEQ ID NO: 10 - DNA, 10894 bp, synthetic construct
  source 1..10894: mol_type = other DNA
  misc_feature 1..10894: note = pMT2649
gaagaagggcgtaaataagaagctataatagcttaaaggtataattacctaacttagtaagcatactatattagccactatactataaagggctcctagaacgtaactacctatagcttagctaccactagctactataagggctagcgcccctcctagcttaatcgcctacctagtcgtatacctaccgcctacgactactagctagaaacgactacggctagaaacgctagcataggagagcccctcacgtaagagggctactagcgcgctctatagtaaccgtaccctagccagggacacctaggccagagctaggactctcgtacacctacagtaggagcgcccgtcagcactatccgtattcctagacaacctaggcctggctagtagtaagaaggactgacggataagcggatagatacgaccgtaggatagaggaacgattgtaaggacggtcataatagataaaagactatatttactaagttataacgggtcgtcatttttccttttgaaccctggtacctattatagcatttggctatctataggttagagctaactaacttactaatagggataataagctagggtcaatatatatttagatagcttctctaagtgcctctagtatttagaacaattaacgcttttattacctacttctgtagaccgtaacaattagtctaggagtgccgcttatagcgcgaatactgctgtggtatcatacctttacagcctctctaagctgtactgattatagccaccgagctatagcacaaatagctcgcttgacttggactttgatcaactacgatgcgaaaaagaaaagcgagaggaaaatgggaagaaaaaaatttttttaaaaaaaatgaaccatataatttaattagcaccgggattggctgttcgttgcctcttctagcttggttggggagagcggacggaatcccaaaatttccggcacgtatggtcgtttgtaattgggtctaccaaaagttgattagtagtgctcgcctgttgaagttaaggcgcctagaggcatataatattactaataatagtccttacactcttggggtccataaaccgtgtatccctgcgccgatgaatgtgctctccatttgagcaaatagaatagtcgacagcaccacgaattaacaggataaagtacggtatgatgggtcagttcagcaatgtgtgagtagtgtactgtggtaagaaggtaggcggtagagagcgggctctccagaccacagaaagagggctccgtcccacaaaggcccagacccacttccaggggtatttatctcaactacatgcaactgggacatacagacaaacgcgaactacgttaaagtgagaactgaagaggtcaaggcatcaagatggttcacatggtctgaaaagaagccattttgttacgtgaaaaaaaaggtttatgtactggtggagcgaagcactccagagtgaaaaataaaactatatacagaagaatcgcgcgtgtctcagaacctccttcagagaggttcgtcgtaataatgggcgctttctactctggacttgcctctttgtgctttacattgggggtcgagcctttccaatcagaagatgcacttaccaaagtagcgctaggctacacagcgctttacccccgaatcaggcgtggggtcactaccccgacgcccttttttttttttttttttgtattcacagagcggtttgcacaatctagatttgttcctcccgcctagtcggtcgccttgtacttggcccggtactcctccgtcctcctgatgtcctcctcggatgcgagccccttgataccctcgatgatgtcgtccaaggtgagaatcgcaaagatgggcagaccatactccttccggagctcaccgatcgcgctcggcatcggcttgctatcgtcgccatccggcgaaggcagcttctccatgcggtccagcgcgaccacgatgccggctacgataccgccctccttctcaatcttggcgatcgcctcacgcttcgccgtgccagccgtgatcacatcatccacgataagcaccctcttgcccttgagcggcgcccccacaatgttgccgccctcaccgtggtcctttgcctccttgcggtcgaacgagtagcacgtggtatggccgtacgtggccggatccagctggcccagccgaatggtggccgccgtcgccaacgggatccccttgtaggccgggccgaagacgatgtcgaactggatcgcgccgctccggtgcgcctcgatgatggcgtgcgcgtacgccgtcgcgagcgcctcgagcagatcggcccggtagaagtctcccgcgttgaagaagtagggcgagatgcgcttcgacttgagctcgaagctgccaaacttgaggatgttgccggcgatggccgcccggaggaagtcggctttgtaagaagcgagtggggccatgtctgtcggttcgtacgtgtcttacgggttcttgggctggaagtttatttgttaccggagggaactcctggtgaagattcgaaaaaaagaaatgagggagttttgtcaaacgtccccctatcgcagagctgaagagagcccctccatcgcgaatttcttggtggggttggattcatggtggggcccaagggtcgttaaacttttttgtatgtctacggagttacacagtactccgtacgtcgtaccccacttgcaatgcttctcgagctgggcttgggatattttgataacgcacaccgcctgccttatcttatcattttgtgctctccgcaaaaaattatttcgggcgacttggggacggcaaatcgacaccgccccgccaggtgctccctatctgacaactcccaaacacccccatcgccaaattcggacagcaccctgcgcgttgcctgccatggcctggaaggatatcgcgcctgtgcctacgtaagggttcaattcgtatatatgctgttcctttcctgaactaagctgatcattttcgtacagggcacaggagtttatcgacatcatcctttcgtaagtttgaaggctaggttatacgtagcggccgctcgatgtgaaatcctcgggcattaggcactagagtttagcgaaaaggtttttacagtactcggtagttcgtcgcctttttcgtgccgctgacggggagcgtttccaagttttggtgaccaaatcagtgactgaacgccaagaaactcctgtcaaccaagcaggcgcaccgctgaacccgcccacgtgggtccgacccccgttcactgctttcgatgaggtggttccatggaccaaggaaacacttgatggcgctcgtcaccagaacgacgaaggcttctcgacttcgatctcaatgtcgttccctatatgtactgtactgatacatacataccacgtaaaggtgccccttagcacgtcctgcagcaatcctcagagctccgctctcatcagatgcataccgcaattgataagtggctacggagtagttacgggcgacatatcaagcgtagcgaccttgattcccagtaccgagcctatacctattgaaataatctgcattatcccatcacagatcctccttagccaggcttacctggggccgcttctggagcttcacattcccatccaacccgctgccgctgtaaactgcgaatgggttcattgtattttttttttgttgttgttttgaatagaaaactctgattccgtatcacccattcatacaaaccactagtcccttattcgaggcgaccggctcctctctgcagaagatgcctcctctctcccacagtcacgggccagagaactcgaaaattctcgcagtacaagatccccctagagattagtctaatttaactcgaatcgcgaccataaccgagttgatggatattcaccctacacgcccaatgctcgagcttgctcgacgatgcgtctcgtggaaagttaggcacctcccccctttcggggcgagtagccgaagcgggggcatcaactgattaacaggcttgttaaaggaagtcttcacggtcggcatacaaatcggccaagacgttgaagatgccataatacataatatgtatgtacaataaccgggcacatacggtgcaactgctagtcagcaattgcgcctcgctttttgacacagagggcaatgcagaacgtcgaatcgaccgagtcagttgctgttggcgtcatgatggctctagggtcagacagaaggtcagagaaagcatgccttagctcgaagccgctcagatgtaattacgcaacgctcggcgttcgagtttacggaggacgacggctacaagatggggctgcttaaagttaccttaaatagaaaatagtgcctggcttaagagatcatgtccgcggggctagcaaggatgtcgggtcttaactcgacggctcgcctagatttcgtgaaaagggaactcactccccgacaggcccgcaagtgaatatgtaattactcaatggaagttctcgaaacggagtccagaaatgatgtggttctgtgggaatgcggcaagaggcgacgttgccgtgaatgcgtgaacattcccgcctcttcttcttctcgtcttcttccttcttcttctttcgggtcgcggatggttgacggccagcgtgcgcacggctgcgtgttatcgagcgtcggtacgtctagccaacatcccgtagacacgacgaccaagcgtcttgagaatgcaacaacgtctcggaacctggcacgcatcttccgccgcaggtcggcagacgccgcctgggcaataccacccctgtccaggccctttccccgcaggcagagccgcgctcttcctttcatggttattcaggaacgtggcttccgagattctcgcctgttctcccccagtcaacctgccgaccgtaacccggttccaccaccgcggactgtccgcaaaacctggttcgcccgagattaatatgctatttccggactaagtgcacaacacacaagcaccccttccgcctcgcgctctagaatctgctttctaacccggttctcgggcccttccctttcgcgacgcctccgctctccttaccaggcaccatccgcaataggtaaggtagccaaccgttttggagcgtgattctgccaaggaccgcatccttgcattcgccatctggtcaaggacccctctttcccgctccattctggtggctctatcgggacggcgttccccatggctctccaggagagtgatgtgcgagtctggagagccggggttggcgtcacgatgctgcccacctagggccggccagcccggcactgcgctcccgttgatccgtctatccccgtcaagagcaccagccccggcgctcgtgaattttcgacttgttcgacttgctacaggtgataaagaggatgcacgccgccctcgatcggcctgtgtggtttctctccctcgtgccaaaccactcccacctcccgccccgagatagttgcttgtttcgctccgtgagagggacacacaccatggtgactccctctctgaagaaggcagctctggctgccctgtccctcttcccgctcctctccctggctagcgaaaccgaaccttccggatatcagctggagaaggtggtcattctctcgcgtcacggtgtccgagcccccaccaagatgacacagacgatgcgcgatgtcactcctcatcagtggcctgagtggcccgtgaagctcggctacatcactcctcgtggagaacacctcatcagcctgatgggcggtttctatagggaacggttccagcagcagggattgcttcccaacgacacctgtccgacccccgacgccgtctacgtgtggaccgacgttaaccagcgtacccgcaagactggagaggctttcctcgccggtcttgcgcctcagtgtgatctggccatccaccaccagcagaacatcacgcaggctgacccgctgtttcacccggtcaaggccggtatctgttcgatgaacaagtctcagacctatgcggctgtcgagaagcaggctggcggccctattgagacgctaaaccagcgctaccaggccgaactggcattgatgtcctctgtgttggatttccccaagtccccatattgccagcagcataacatcggcaaactgtgcgacttttcacaggctatgcctagccgcctcaacatctccgatgacgggaatgaggtgcaactcgaaggcgccgtcggtctttcctccacgctcgccgagatcttcctactggaatacgctcagggtatgcctgtggtcgcctggggcaacattcacaacgagagccagtggaagagcctccttaacttgcacaacgcccatttcaacctgatgcacagaacgccctacattgccaagcaccagggaacccctttacttcaggctatcagcaacgctctcaacccaaatgcaactgagtcgaagctccccgatatctctcccgacaacaagatccttttcattgccggccacgacaccaacatcgcaaacatcggaggcatgttgggtatgaactggactctcccgggccagccagacaatactccgcccggcggtggactggttttcgaactctggcagaacccggataaccatcagcagtacgttgcggtgaagatgatctaccagaccatggaccagctgcgcaattccgagaagctggacttgaagagcaaccctgctgggatcgtccccattgagatcgaaggttgcgagaacatcggtaccgacaagctgtgccagctggatacttttcagaagcgtgttgcccaggtcattgagcccgcgtgccaaatctaatagcacgaacctctctgaaggaggttctgagacacgcgcgattcttctgtatatagttttatttttcactctggagtgcttcgctccaccagtacataaacctttttttcacgtaacaaaatggcttcttttcagaccatgtgaaccatcttgatgccttgacctcttcagttctcactttaacgtagttcgcgtttgtctgtatgtcccagatctacgtacaagacgagtgtgccagtcagccatcgaatgatgggtcgtcatggacaacggagcaatgccatctcgggactggggccaccgacctgccggcagggggaggagaaaaaaaagggggcggcgtcggcaagatggcaagaggtctgggagatagtcaaggacgaagggccaagcgggtcgtgtcgcttgcactctggtgaggctcgctagtgcttgcggtgggtgaggtggatcagagtagaggcccgatggagaatgggcgctgcggtgacaagcctaaccggtcgatagcggctagtaggctgggcggcgatgtcacgttatggttgccacggccgggcacggactgagggcatcgttcgaggacgtggagccgtgcgatggaagcgattaaggttgctgaagggtcggcgagggcaaccacacacctcattggaggagtctgggcgcggcagccaggttatcaggggtgacacggaaggcgtccgccgagtgcgactctgcgaagcccaggccgccctgcggaagaaccttggttcacgatgctctctgatgaccgcagaagagcgtatttgacgctccgcgcaaatgcgatccgaagtgccggtgcgaggctcctgtcgcctgctcgtctccgccctgactctcaacgcagcacgaacagcgctgccgtagatactccttcggagcctaccctggtgtacactagattcaagcaaaaggcgcacgagagcggagaaagccgcagcacaccccatcccttcccttcgcaacggatctcgcttctccccgcattttagtgtagtcccggtggcccacttaccccggcttctgcagtcttggtatttggtggtttctgatgtgcggggagagaggccttcagaaaaaaatgtgtctcttcgtggaactcgccgtgtgtgcgactttaacttgtatggaagttgaggtcgatttccccagatcttgaggctcaaaaactcccaaatttggcaccaaacgactgggacgcaaaaccgggtacctggtgccatcttcaatatacttgggaatgcagccgcggtgattgtcggtctgggcgcaacctcatcggcgttggcgggcgatcagcaggtgcaacggggggtgcaaaccgggaaatgagaagagcccagaccaacgaggaccgcggcatcgacctccgatcaccctaagttgggcctagtgccgccccaccaaggaacggaccagggtagtttgctcctcccgatcactggtagtcgtgccacagtgcagcgcattcaagggcaagccgctttcccaaatagagcaggaagggctggtttcaactttgaggtcaggacatccgtaacgttattccactttcggtgttctctttcttccgcagcttcttcggcagcgagttagttgcgcaactctcgtcacttttgcaatactttcatcgacgttccccaaatcggatacctcatcgatgctcttctgcgttaattaacaattgggatcctctagacccgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgctcgatttaaatctcgagaggcctgacgtcgggcccggtaccacgcgtcatatgactagt

SEQ ID NO: 11 - DNA, 10468 bp, synthetic construct
  source 1..10468: mol_type = other DNA
  misc_feature 1..10468: note = pMT2652
gaagaagggcgtaaataagaagctataatagcttaaaggtataattacctaacttagtaagcatactatattagccactatactataaagggctcctagaacgtaactacctatagcttagctaccactagctactataagggctagcgcccctcctagcttaatcgcctacctagtcgtatacctaccgcctacgactactagctagaaacgactacggctagaaacgctagcataggagagcccctcacgtaagagggctactagcgcgctctatagtaaccgtaccctagccagggacacctaggccagagctaggactctcgtacacctacagtaggagcgcccgtcagcactatccgtattcctagacaacctaggcctggctagtagtaagaaggactgacggataagcggatagatacgaccgtaggatagaggaacgattgtaaggacggtcataatagataaaagactatatttactaagttataacgggtcgtcatttttccttttgaaccctggtacctattatagcatttggctatctataggttagagctaactaacttactaatagggataataagctagggtcaatatatatttagatagcttctctaagtgcctctagtatttagaacaattaacgcttttattacctacttctgtagaccgtaacaattagtctaggagtgccgcttatagcgcgaatactgctgtggtatcatacctttacagcctctctaagctgtactgattatagccaccgagctatagcacaaatagctcgcttgacttggactttgatcaactacgatgcgaaaaagaaaagcgagaggaaaatgggaagaaaaaaatttttttaaaaaaaatgaaccatataatttaattagcaccgggattggctgttcgttgcctcttctagcttggttggggagagcggacggaatcccaaaatttccggcacgtatggtcgtttgtaattgggtctaccaaaagttgattagtagtgctcgcctgttgaagttaaggcgcctagaggcatataatattactaataatagtccttacactcttggggtccataaaccgtgtatccctgcgccgatgaatgtgctctccatttgagcaaatagaatagtcgacagcaccacgaattaacaggataaagtacggtatgatgggtcagttcagcaatgtgtgagtagtgtactgtggtaagaaggtaggcggtagagagcgggctctccagaccacagaaagagggctccgtcccacaaaggcccagacccacttccaggggtatttatctcaactacatgcaactgggacatacagacaaacgcgaactacgttaaagtgagaactgaagaggtcaaggcatcaagatggttcacatggtctgaaaagaagccattttgttacgtgaaaaaaaaggtttatgtactggtggagcgaagcactccagagtgaaaaataaaactatatacagaagaatcgcgcgtgtctcagaacctccttcagagaggttcgtcgtaataatgggcgctttctactctggacttgcctctttgtgctttacattgggggtcgagcctttccaatcagaagatgcacttaccaaagtagcgctaggctacacagcgctttacccccgaatcaggcgtggggtcactaccccgacgcccttttttttttttttttttgtattcacagagcggtttgcacaatctagatttgttcctcccgcctagtcggtcgccttgtacttggcccggtactcctccgtcctcctgatgtcctcctcggatgcgagccccttgataccctcgatgatgtcgtccaaggtgagaatcgcaaagatgggcagaccatactccttccggagctcaccgatcgcgctcggcatcggcttgctatcgtcgccatccggcgaaggcagcttctccatgcggtccagcgcgaccacgatgccggctacgataccgccctccttctcaatcttggcgatcgcctcacgcttcgccgtgccagccgtgatcacatcatccacgataagcaccctcttgcccttgagcggcgcccccacaatgttgccgccctcaccgtggtcctttgcctccttgcggtcgaacgagtagcacgtggtatggccgtacgtggccggatccagctggcccagccgaatggtggccgccgtcgccaacgggatccccttgtaggccgggccgaagacgatgtcgaactggatcgcgccgctccggtgcgcctcgatgatggcgtgcgcgtacgccgtcgcgagcgcctcgagcagatcggcccggtagaagtctcccgcgttgaagaagtagggcgagatgcgcttcgacttgagctcgaagctgccaaacttgaggatgttgccggcgatggccgcccggaggaagtcggctttgtaagaagcgagtggggccatgtctgtcggttcgtacgtgtcttacgggttcttgggctggaagtttatttgttaccggagggaactcctggtgaagattcgaaaaaaagaaatgagggagttttgtcaaacgtccccctatcgcagagctgaagagagcccctccatcgcgaatttcttggtggggttggattcatggtggggcccaagggtcgttaaacttttttgtatgtctacggagttacacagtactccgtacgtcgtaccccacttgcaatgcttctcgagctgggcttgggatattttgataacgcacaccgcctgccttatcttatcattttgtgctctccgcaaaaaattatttcgggcgacttggggacggcaaatcgacaccgccccgccaggtgctccctatctgacaactcccaaacacccccatcgccaaattcggacagcaccctgcgcgttgcctgccatggcctggaaggatatcgcgcctgtgcctacgtaagggttcaattcgtatatatgctgttcctttcctgaactaagctgatcattttcgtacagggcacaggagtttatcgacatcatcctttcgtaagtttgaaggctaggttatacgtagcggccgctcgaacctcacgggcagaagctggaggcgcccgggagagagtggatcgagttgcatgcgcaagcgcccttcatctcttcagccgagaatctgttgcctaattacatagtagatgctgggaacagtctgagaagcctgcgcaggttcttcattaattaccaagaacgcgggagaggtgggaagacgccgaaaactcgacgcggaccattcgctgttttccgatcttacaaaaaaaaggtatccgatttggggaacgtcgatgaaagtattgcaaaagtgacgagagttgcgcaactaactcgctgccgaagaagctgcggaagaaagagaacaccgaaagtggaataacgttacggatgtcctgacctcaaagttgaaaccagcccttcctgctctatttgggaaagcggcttgcccttgaatgcgctgcactgtggcacgactaccagtgatcgggaggagcaaactaccctggtccgttccttggtggggcggcactaggcccaacttagggtgatcggaggtcgatgccgcggtcctcgttggtctgggctcttctcatttcccggtttgcaccccccgttgcacctgctgatcgcccgccaacgccgatgaggttgcgcccagaccgacaatcaccgcggctgcattcccaagtatattgaagatggcaccaggtacccggttttgcgtcccagtcgtttggtgccaaatttgggagtttttgagcctcaagatctggggaaatcgacctcaacttccatacaagttaaagtcgcacacacggcgagttccacgaagagacacatttttttctgaaggcctctctccccgcacatcagaaaccaccaaataccaagactgcagaagccggggtaagtgggccaccgggactacactaaaatgcggggagaagcgagatccgttgcgaagggaagggatggggtgtgctgcggctttctccgctctcgtgcgccttttgcttgaatctagtgtacaccagggtaggctccgaaggagtatctacggcagcgctgttcgtgctgcgttgagagtcagggcggagacgagcaggcgacaggagcctcgcaccggcacttcggatcgcatttgcgcggagcgtcaaatacgctcttctgcggtcatcagagagcatcgtgaaccaaggttcttccgcagggcggcctgggcttcgcagagtcgcactcggcggacgccttccgtgtcacccctgataacctggctgccgcgcccagactcctccaatgaggtgtgtggttgccctcgccgacccttcagcaaccttaatcgcttccatcgcacggctccacgtcctcgaacgatgccctcagtccgtgcccggccgtggcaaccataacgtgacatcgccgcccagcctactagccgctatcgaccggttaggcttgtcaccgcagcgcccattctccatcgggcctctactctgatccacctcacccaccgcaagcactagcgagcctcaccagagtgcaagcgacacgacccgcttggcccttcgtccttgactatctcccagacctcttgccatcttgccgacgccgcccccttttttttctcctccccctgccggcaggtcggtggccccagtcccgagatggcattgctccgttgtccatgacgacccatcattcgatggctgactggcacactcgtcttgtttgagcatcgacggcccgcggcccgtctcccacggtacggaacctcgttgtacagtacctctcgtaatgatacccaacaccggggccgagcgctgggagggcggcgttcccgagaagccgggaaggcggctggccggctgacctttgtgacttggcgatggatgcggccatggagaatgtccgtccgaagcgacgcgacaattagcctggctaccatcgatataaattgggtgattcccagctcttgatgggcgtgtcttctgcctggcagccctcgtcttcagatcaagcaactgtgtgctgatcctcttccgccatggtgactccctctctgaagaaggcagctctggctgccctgtccctcttcccgctcctctccctggctagcgaaaccgaaccttccggatatcagctggagaaggtggtcattctctcgcgtcacggtgtccgagcccccaccaagatgacacagacgatgcgcgatgtcactcctcatcagtggcctgagtggcccgtgaagctcggctacatcactcctcgtggagaacacctcatcagcctgatgggcggtttctatagggaacggttccagcagcagggattgcttcccaacgacacctgtccgacccccgacgccgtctacgtgtggaccgacgttaaccagcgtacccgcaagactggagaggctttcctcgccggtcttgcgcctcagtgtgatctggccatccaccaccagcagaacatcacgcaggctgacccgctgtttcacccggtcaaggccggtatctgttcgatgaacaagtctcagacctatgcggctgtcgagaagcaggctggcggccctattgagacgctaaaccagcgctaccaggccgaactggcattgatgtcctctgtgttggatttccccaagtccccatattgccagcagcataacatcggcaaactgtgcgacttttcacaggctatgcctagccgcctcaacatctccgatgacgggaatgaggtgcaactcgaaggcgccgtcggtctttcctccacgctcgccgagatcttcctactggaatacgctcagggtatgcctgtggtcgcctggggcaacattcacaacgagagccagtggaagagcctccttaacttgcacaacgcccatttcaacctgatgcacagaacgccctacattgccaagcaccagggaacccctttacttcaggctatcagcaacgctctcaacccaaatgcaactgagtcgaagctccccgatatctctcccgacaacaagatccttttcattgccggccacgacaccaacatcgcaaacatcggaggcatgttgggtatgaactggactctcccgggccagccagacaatactccgcccggcggtggactggttttcgaactctggcagaacccggataaccatcagcagtacgttgcggtgaagatgatctaccagaccatggaccagctgcgcaattccgagaagctggacttgaagagcaaccctgctgggatcgtccccattgagatcgaaggttgcgagaacatcggtaccgacaagctgtgccagctggatacttttcagaagcgtgttgcccaggtcattgagcccgcgtgccaaatctaatagcacgaacctctctgaaggaggttctgagacacgcgcgattcttctgtatatagttttatttttcactctggagtgcttcgctccaccagtacataaacctttttttcacgtaacaaaatggcttcttttcagaccatgtgaaccatcttgatgccttgacctcttcagttctcactttaacgtagttcgcgtttgtctgtatgtcccagatctacgtacaagacgagtgtgccagtcagccatcgaatgatgggtcgtcatggacaacggagcaatgccatctcgggactggggccaccgacctgccggcagggggaggagaaaaaaaagggggcggcgtcggcaagatggcaagaggtctgggagatagtcaaggacgaagggccaagcgggtcgtgtcgcttgcactctggtgaggctcgctagtgcttgcggtgggtgaggtggatcagagtagaggcccgatggagaatgggcgctgcggtgacaagcctaaccggtcgatagcggctagtaggctgggcggcgatgtcacgttatggttgccacggccgggcacggactgagggcatcgttcgaggacgtggagccgtgcgatggaagcgattaaggttgctgaagggtcggcgagggcaaccacacacctcattggaggagtctgggcgcggcagccaggttatcaggggtgacacggaaggcgtccgccgagtgcgactctgcgaagcccaggccgccctgcggaagaaccttggttcacgatgctctctgatgaccgcagaagagcgtatttgacgctccgcgcaaatgcgatccgaagtgccggtgcgaggctcctgtcgcctgctcgtctccgccctgactctcaacgcagcacgaacagcgctgccgtagatactccttcggagcctaccctggtgtacactagattcaagcaaaaggcgcacgagagcggagaaagccgcagcacaccccatcccttcccttcgcaacggatctcgcttctccccgcattttagtgtagtcccggtggcccacttaccccggcttctgcagtcttggtatttggtggtttctgatgtgcggggagagaggccttcagaaaaaaatgtgtctcttcgtggaactcgccgtgtgtgcgactttaacttgtatggaagttgaggtcgatttccccagatcttgaggctcaaaaactcccaaatttggcaccaaacgactgggacgcaaaaccgggtacctggtgccatcttcaatatacttgggaatgcagccgcggtgattgtcggtctgggcgcaacctcatcggcgttggcgggcgatcagcaggtgcaacggggggtgcaaaccgggaaatgagaagagcccagaccaacgaggaccgcggcatcgacctccgatcaccctaagttgggcctagtgccgccccaccaaggaacggaccagggtagtttgctcctcccgatcactggtagtcgtgccacagtgcagcgcattcaagggcaagccgctttcccaaatagagcaggaagggctggtttcaactttgaggtcaggacatccgtaacgttattccactttcggtgttctctttcttccgcagcttcttcggcagcgagttagttgcgcaactctcgtcacttttgcaatactttcatcgacgttccccaaatcggatacctcatcgatgctcttctgcgttaattaacaattgggatcctctagacccgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgctcgatttaaatctcgagaggcctgacgtcgggcccggtaccacgcgtcatatgactagt

SEQ ID NO: 12 - DNA, 9974 bp, synthetic construct
  source 1..9974: mol_type = other DNA
  misc_feature 1..9974: note = pMT2196
gaagaagggcgtaaataagaagctataatagcttaaaggtataattacctaacttagtaagcatactatattagccactatactataaagggctcctagaacgtaactacctatagcttagctaccactagctactataagggctagcgcccctcctagcttaatcgcctacctagtcgtatacctaccgcctacgactactagctagaaacgactacggctagaaacgctagcataggagagcccctcacgtaagagggctactagcgcgctctatagtaaccgtaccctagccagggacacctaggccagagctaggactctcgtacacctacagtaggagcgcccgtcagcactatccgtattcctagacaacctaggcctggctagtagtaagaaggactgacggataagcggatagatacgaccgtaggatagaggaacgattgtaaggacggtcataatagataaaagactatatttactaagttataacgggtcgtcatttttccttttgaaccctggtacctattatagcatttggctatctataggttagagctaactaacttactaatagggataataagctagggtcaatatatatttagatagcttctctaagtgcctctagtatttagaacaattaacgcttttattacctacttctgtagaccgtaacaattagtctaggagtgccgcttatagcgcgaatactgctgtggtatcatacctttacagcctctctaagctgtactgattatagccaccgagctatagcacaaatagctcgcttgacttggactttgatcaactacgatgcgaaaaagaaaagcgagaggaaaatgggaagaaaaaaatttttttaaaaaaaatgaaccatataatttaattagcaccgggattggctgttcgttgcctcttctagcttggttggggagagcggacggaatcccaaaatttccggcacgtatggtcgtttgtaattgggtctaccaaaagttgattagtagtgctcgcctgttgaagttaaggcgcctagaggcatataatattactaataatagtccttacactcttggggtccataaaccgtgtatccctgcgccgatgaatgtgctctccatttgagcaaatagaatagtcgacagcaccacgaattaacaggataaagtacggtatgatgggtcagttcagcaatgtgtgagtagtgtactgtggtaagaaggtaggcggtagagagcgggctctccagaccacagaaagagggctccgtcccacaaaggcccagacccacttccaggggtatttatctcaactacatgcaactgggacatacagacaaacgcgaactacgttaaagtgagaactgaagaggtcaaggcatcaagatggttcacatggtctgaaaagaagccattttgttacgtgaaaaaaaaggtttatgtactggtggagcgaagcactccagagtgaaaaataaaactatatacagaagaatcgcgcgtgtctcagaacctccttcagagaggttcgtcgtaataatgggcgctttctactctggacttgcctctttgtgctttacattgggggtcgagcctttccaatcagaagatgcacttaccaaagtagcgctaggctacacagcgctttacccccgaatcaggcgtggggtcactaccccgacgcccttttttttttttttttttgtattcacagagcggtttgcacaatctagatttgttcctcccgcctagtcggtcgccttgtacttggcccggtactcctccgtcctcctgatgtcctcctcggatgcgagccccttgataccctcgatgatgtcgtccaaggtgagaatcgcaaagatgggcagaccatactccttccggagctcaccgatcgcgctcggcatcggcttgctatcgtcgccatccggcgaaggcagcttctccatgcggtccagcgcgaccacgatgccggctacgataccgccctccttctcaatcttggcgatcgcctcacgcttcgccgtgccagccgtgatcacatcatccacgataagcaccctcttgcccttgagcggcgcccccacaatgttgccgccctcaccgtggtcctttgcctccttgcggtcgaacgagtagcacgtggtatggccgtacgtggccggatccagctggcccagccgaatggtggccgccgtcgccaacgggatccccttgtaggccgggccgaagacgatgtcgaactggatcgcgccgctccggtgcgcctcgatgatggcgtgcgcgtacgccgtcgcgagcgcctcgagcagatcggcccggtagaagtctcccgcgttgaagaagtagggcgagatgcgcttcgacttgagctcgaagctgccaaacttgaggatgttgccggcgatggccgcccggaggaagtcggctttgtaagaagcgagtggggccatgtctgtcggttcgtacgtgtcttacgggttcttgggctggaagtttatttgttaccggagggaactcctggtgaagattcgaaaaaaagaaatgagggagttttgtcaaacgtccccctatcgcagagctgaagagagcccctccatcgcgaatttcttggtggggttggattcatggtggggcccaagggtcgttaaacttttttgtatgtctacggagttacacagtactccgtacgtcgtaccccacttgcaatgcttctcgagctgggcttgggatattttgataacgcacaccgcctgccttatcttatcattttgtgctctccgcaaaaaattatttcgggcgacttggggacggcaaatcgacaccgccccgccaggtgctccctatctgacaactcccaaacacccccatcgccaaattcggacagcaccctgcgcgttgcctgccatggcctggaaggatatcgcgcctgtgcctacgtaagggttcaattcgtatatatgctgttcctttcctgaactaagctgatcattttcgtacagggcacaggagtttatcgacatcatcctttcgtaagtttgaaggctaggttatacgtagcggccgctcgaagactaattacatgaaaactgccaggactacatggagccttaggtagcaaagagaggctaccctcctttcaacgcaacaccatagttaagtagagcccaaagtcgcgctcgtcgatgactacccgcccccacgggctactttagcgtgacaaggtcttccgcgcccatggttcggatttggatcggtagcatacaacgcacttacgtcattttcgattattgccgacgcttgaggtcctaggtacctccatttggcaccatcattgacttgctacgggaagggtaagtcaggtacacctagtgtaactgtttcgggatgacgagcaaagccggctgccatgaaccgttcgtacacagcacaccagtaccgcgtctccaaaaacggccgtggctgtgtgtccctggttagctcctcagtcttgagctcgatggttgctaattatgtgtagcgcgcgtcggtagcgacccaatcccctccccccttctctcctggcaagaagtcgtcacatctcgggagctcccctgcctggcacgcggcggaagtacgggacggagggccaccaagttgtcctggtattcggaccggttcaggttggcagcgaatccgcgcatcggaggaatggtgaagaaggaagggggccttaaggggtattgccttgtggagcacgtatcatgcgccatcgcacaccacacaccgcggacagcggttgtgtttctcttggactgcaaggacagcaaagcatgcaggtacctgagtgtccaggactgcctaaatacattggcgcccggacgtgtggatcgaccgagggtccccggctcccgagggaatgggaaatctaggtcgggcacacgttgtatgcgcgcaggagttcatcaagaatgacaaaggccatgagcgagagatgacgcagcggccggagactcggttcgaaataatagtctctacttctgcttcacttgaagaaccgagactctcaccgcgtcgcctgcgcgggattcatgcactccacacggcgcccgcaggagtcgtttgactggaccgcgggccactgggcatggccagctgggcggcggagccaaatgccgtcgatgtgtttggaatcgtcaacaatatcggcgtggcgcctgagtggtcgccgctgcattttagccggccgggccaaggcggcagagcgctcccgaaacccaggacggtgtaaccctggctggaatccgggaatgccaggtcttgccaaggagagtaagtcggggccatggaccggaaaccttctcgatctggggaggatttgcaaataccgacgacgagcggccggcggccggccgggggtggggggaaaggggagaaagcagagaagcaggctccttggccaggggccagggcatataaggacgggaataaggcctcgtagcgaagcggcgttctgaaaaggaggaacagaactaacgaagacgacccaagcataccaaggtcgtgttgattagcacccccgattcttccagttctttccgccatctgccatggtgactccctctctgaagaaggcagctctggctgccctgtccctcttcccgctcctctccctggctagcgaaaccgaaccttccggatatcagctggagaaggtggtcattctctcgcgtcacggtgtccgagcccccaccaagatgacacagacgatgcgcgatgtcactcctcatcagtggcctgagtggcccgtgaagctcggctacatcactcctcgtggagaacacctcatcagcctgatgggcggtttctatagggaacggttccagcagcagggattgcttcccaacgacacctgtccgacccccgacgccgtctacgtgtggaccgacgttaaccagcgtacccgcaagactggagaggctttcctcgccggtcttgcgcctcagtgtgatctggccatccaccaccagcagaacatcacgcaggctgacccgctgtttcacccggtcaaggccggtatctgttcgatgaacaagtctcagacctatgcggctgtcgagaagcaggctggcggccctattgagacgctaaaccagcgctaccaggccgaactggcattgatgtcctctgtgttggatttccccaagtccccatattgccagcagcataacatcggcaaactgtgcgacttttcacaggctatgcctagccgcctcaacatctccgatgacgggaatgaggtgcaactcgaaggcgccgtcggtctttcctccacgctcgccgagatcttcctactggaatacgctcagggtatgcctgtggtcgcctggggcaacattcacaacgagagccagtggaagagcctccttaacttgcacaacgcccatttcaacctgatgcacagaacgccctacattgccaagcaccagggaacccctttacttcaggctatcagcaacgctctcaacccaaatgcaactgagtcgaagctccccgatatctctcccgacaacaagatccttttcattgccggccacgacaccaacatcgcaaacatcggaggcatgttgggtatgaactggactctcccgggccagccagacaatactccgcccggcggtggactggttttcgaactctggcagaacccggataaccatcagcagtacgttgcggtgaagatgatctaccagaccatggaccagctgcgcaattccgagaagctggacttgaagagcaaccctgctgggatcgtccccattgagatcgaaggttgcgagaacatcggtaccgacaagctgtgccagctggatacttttcagaagcgtgttgcccaggtcattgagcccgcgtgccaaatctaatagcacgaacctctctgaaggaggttctgagacacgcgcgattcttctgtatatagttttatttttcactctggagtgcttcgctccaccagtacataaacctttttttcacgtaacaaaatggcttcttttcagaccatgtgaaccatcttgatgccttgacctcttcagttctcactttaacgtagttcgcgtttgtctgtatgtcccagatctacgtacaagacgagtgtgccagtcagccatcgaatgatgggtcgtcatggacaacggagcaatgccatctcgggactggggccaccgacctgccggcagggggaggagaaaaaaaagggggcggcgtcggcaagatggcaagaggtctgggagatagtcaaggacgaagggccaagcgggtcgtgtcgcttgcactctggtgaggctcgctagtgcttgcggtgggtgaggtggatcagagtagaggcccgatggagaatgggcgctgcggtgacaagcctaaccggtcgatagcggctagtaggctgggcggcgatgtcacgttatggttgccacggccgggcacggactgagggcatcgttcgaggacgtggagccgtgcgatggaagcgattaaggttgctgaagggtcggcgagggcaaccacacacctcattggaggagtctgggcgcggcagccaggttatcaggggtgacacggaaggcgtccgccgagtgcgactctgcgaagcccaggccgccctgcggaagaaccttggttcacgatgctctctgatgaccgcagaagagcgtatttgacgctccgcgcaaatgcgatccgaagtgccggtgcgaggctcctgtcgcctgctcgtctccgccctgactctcaacgcagcacgaacagcgctgccgtagatactccttcggagcctaccctggtgtacactagattcaagcaaaaggcgcacgagagcggagaaagccgcagcacaccccatcccttcccttcgcaacggatctcgcttctccccgcattttagtgtagtcccggtggcccacttaccccggcttctgcagtcttggtatttggtggtttctgatgtgcggggagagaggccttcagaaaaaaatgtgtctcttcgtggaactcgccgtgtgtgcgactttaacttgtatggaagttgaggtcgatttccccagatcttgaggctcaaaaactcccaaatttggcaccaaacgactgggacgcaaaaccgggtacctggtgccatcttcaatatacttgggaatgcagccgcggtgattgtcggtctgggcgcaacctcatcggcgttggcgggcgatcagcaggtgcaacggggggtgcaaaccgggaaatgagaagagcccagaccaacgaggaccgcggcatcgacctccgatcaccctaagttgggcctagtgccgccccaccaaggaacggaccagggtagtttgctcctcccgatcactggtagtcgtgccacagtgcagcgcattcaagggcaagccgctttcccaaatagagcaggaagggctggtttcaactttgaggtcaggacatccgtaacgttattccactttcggtgttctctttcttccgcagcttcttcggcagcgagttagttgcgcaactctcgtcacttttgcaatactttcatcgacgttccccaaatcggatacctcatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgctcgatttaaatctcgagaggcctgacgtcgggcccggtaccacgcgtcatatgactagt

SEQ ID NO: 13 - DNA, 10736 bp, synthetic construct
  source 1..10736: mol_type = other DNA
  misc_feature 1..10736: note = pMT2653
gaagaagggcgtaaataagaagctataatagcttaaaggtataattacctaacttagtaagcatactatattagccactatactataaagggctcctagaacgtaactacctatagcttagctaccactagctactataagggctagcgcccctcctagcttaatcgcctacctagtcgtatacctaccgcctacgactactagctagaaacgactacggctagaaacgctagcataggagagcccctcacgtaagagggctactagcgcgctctatagtaaccgtaccctagccagggacacctaggccagagctaggactctcgtacacctacagtaggagcgcccgtcagcactatccgtattcctagacaacctaggcctggctagtagtaagaaggactgacggataagcggatagatacgaccgtaggatagaggaacgattgtaaggacggtcataatagataaaagactatatttactaagttataacgggtcgtcatttttccttttgaaccctggtacctattatagcatttggctatctataggttagagctaactaacttactaatagggataataagctagggtcaatatatatttagatagcttctctaagtgcctctagtatttagaacaattaacgcttttattacctacttctgtagaccgtaacaattagtctaggagtgccgcttatagcgcgaatactgctgtggtatcatacctttacagcctctctaagctgtactgattatagccaccgagctatagcacaaatagctcgcttgacttggactttgatcaactacgatgcgaaaaagaaaagcgagaggaaaatgggaagaaaaaaatttttttaaaaaaaatgaaccatataatttaattagcaccgggattggctgttcgttgcctcttctagcttggttggggagagcggacggaatcccaaaatttccggcacgtatggtcgtttgtaattgggtctaccaaaagttgattagtagtgctcgcctgttgaagttaaggcgcctagaggcatataatattactaataatagtccttacactcttggggtccataaaccgtgtatccctgcgccgatgaatgtgctctccatttgagcaaatagaatagtcgacagcaccacgaattaacaggataaagtacggtatgatgggtcagttcagcaatgtgtgagtagtgtactgtggtaagaaggtaggcggtagagagcgggctctccagaccacagaaagagggctccgtcccacaaaggcccagacccacttccaggggtatttatctcaactacatgcaactgggacatacagacaaacgcgaactacgttaaagtgagaactgaagaggtcaaggcatcaagatggttcacatggtctgaaaagaagccattttgttacgtgaaaaaaaaggtttatgtactggtggagcgaagcactccagagtgaaaaataaaactatatacagaagaatcgcgcgtgtctcagaacctccttcagagaggttcgtcgtaataatgggcgctttctactctggacttgcctctttgtgctttacattgggggtcgagcctttccaatcagaagatgcacttaccaaagtagcgctaggctacacagcgctttacccccgaatcaggcgtggggtcactaccccgacgcccttttttttttttttttttgtattcacagagcggtttgcacaatctagatttgttcctcccgcctagtcggtcgccttgtacttggcccggtactcctccgtcctcctgatgtcctcctcggatgcgagccccttgataccctcgatgatgtcgtccaaggtgagaatcgcaaagatgggcagaccatactccttccggagctcaccgatcgcgctcggcatcggcttgctatcgtcgccatccggcgaaggcagcttctccatgcggtccagcgcgaccacgatgccggctacgataccgccctccttctcaatcttggcgatcgcctcacgcttcgccgtgccagccgtgatcacatcatccacgataagcaccctcttgcccttgagcggcgcccccacaatgttgccgccctcaccgtggtcctttgcctccttgcggtcgaacgagtagcacgtggtatggccgtacgtggccggatccagctggcccagccgaatggtggccgccgtcgccaacgggatccccttgtaggccgggccgaagacgatgtcgaactggatcgcgccgctccggtgcgcctcgatgatggcgtgcgcgtacgccgtcgcgagcgcctcgagcagatcggcccggtagaagtctcccgcgttgaagaagtagggcgagatgcgcttcgacttgagctcgaagctgccaaacttgaggatgttgccggcgatggccgcccggaggaagtcggctttgtaagaagcgagtggggccatgtctgtcggttcgtacgtgtcttacgggttcttgggctggaagtttatttgttaccggagggaactcctggtgaagattcgaaaaaaagaaatgagggagttttgtcaaacgtccccctatcgcagagctgaagagagcccctccatcgcgaatttcttggtggggttggattcatggtggggcccaagggtcgttaaacttttttgtatgtctacggagttacacagtactccgtacgtcgtaccccacttgcaatgcttctcgagctgggcttgggatattttgataacgcacaccgcctgccttatcttatcattttgtgctctccgcaaaaaattatttcgggcgacttggggacggcaaatcgacaccgccccgccaggtgctccctatctgacaactcccaaacacccccatcgccaaattcggacagcaccctgcgcgttgcctgccatggcctggaaggatatcgcgcctgtgcctacgtaagggttcaattcgtatatatgctgttcctttcctgaactaagctgatcattttcgtacagggcacaggagtttatcgacatcatcctttcgtaagtttgaaggctaggttatacgtagcggccgctcgatggtttgctgcttcagctctcaaataaatctggggtcggaggggtgaaggtgtggagaggggaggtttatcggcatttccatggctgtacgggaagttgttgaaccgcccgagagttgatttccgttattacttattgatttgcagtaatccatagattgcgcgacagtgtccggctgtgtgacttttgtgccctgtcttgacagccaaaccgagagctcaagggtggatgagtgtagggtcggttggtcggtattgcgtgcagtcacggtctgggaaggtggtggagctctttctccggagctccggtcagttccttccaaatgaacgcgattgaaaccacgcgccttccgcagcactgcctcattgctataagaaacttggcggtaccatcctaggtcatttttttttggtgatggatcgatccaaggaatagtctccctccctctctctctctcccccctcccgaaactgtctgaaactgccagtatggaacccaccctccacagctcctggaaacaacattgaccccggcatgagcgaatcaccgcttccacggaccgcttccttacagatagaagcgcatcaataaatgcgcatctgagcaattcgccactttcgagcaacacatgaggtggtgaacgaaaaataaaaggggaagggcggaaagggtgagtgggctgttgttcgaggagatcgaacccagccagcttggctcgggggccgttcccggattttccgggcgtgacgtgactgcgaagactaattacatgaaaactgccaggactacatggagccttaggtagcaaagagaggctaccctcctttcaacgcaacaccatagttaagtagagcccaaagtcgcgctcgtcgatgactacccgcccccacgggctactttagcgtgacaaggtcttccgcgcccatggttcggatttggatcggtagcatacaacgcacttacgtcattttcgattattgccgacgcttgaggtcctaggtacctccatttggcaccatcattgacttgctacgggaagggtaagtcaggtacacctagtgtaactgtttcgggatgacgagcaaagccggctgccatgaaccgttcgtacacagcacaccagtaccgcgtctccaaaaacggccgtggctgtgtgtccctggttagctcctcagtcttgagctcgatggttgctaattatgtgtagcgcgcgtcggtagcgacccaatcccctccccccttctctcctggcaagaagtcgtcacatctcgggagctcccctgcctggcacgcggcggaagtacgggacggagggccaccaagttgtcctggtattcggaccggttcaggttggcagcgaatccgcgcatcggaggaatggtgaagaaggaagggggccttaaggggtattgccttgtggagcacgtatcatgcgccatcgcacaccacacaccgcggacagcggttgtgtttctcttggactgcaaggacagcaaagcatgcaggtacctgagtgtccaggactgcctaaatacattggcgcccggacgtgtggatcgaccgagggtccccggctcccgagggaatgggaaatctaggtcgggcacacgttgtatgcgcgcaggagttcatcaagaatgacaaaggccatgagcgagagatgacgcagcggccggagactcggttcgaaataatagtctctacttctgcttcacttgaagaaccgagactctcaccgcgtcgcctgcgcgggattcatgcactccacacggcgcccgcaggagtcgtttgactggaccgcgggccactgggcatggccagctgggcggcggagccaaatgccgtcgatgtgtttggaatcgtcaacaatatcggcgtggcgcctgagtggtcgccgctgcattttagccggccgggccaaggcggcagagcgctcccgaaacccaggacggtgtaaccctggctggaatccgggaatgccaggtcttgccaaggagagtaagtcggggccatggaccggaaaccttctcgatctggggaggatttgcaaataccgacgacgagcggccggcggccggccgggggtggggggaaaggggagaaagcagagaagcaggctccttggccaggggccagggcatataaggacgggaataaggcctcgtagcgaagcggcgttctgaaaaggaggaacagaactaacgaagacgacccaagcataccaaggtcgtgttgattagcacccccgattcttccagttctttccgccatctgccatggtgactccctctctgaagaaggcagctctggctgccctgtccctcttcccgctcctctccctggctagcgaaaccgaaccttccggatatcagctggagaaggtggtcattctctcgcgtcacggtgtccgagcccccaccaagatgacacagacgatgcgcgatgtcactcctcatcagtggcctgagtggcccgtgaagctcggctacatcactcctcgtggagaacacctcatcagcctgatgggcggtttctatagggaacggttccagcagcagggattgcttcccaacgacacctgtccgacccccgacgccgtctacgtgtggaccgacgttaaccagcgtacccgcaagactggagaggctttcctcgccggtcttgcgcctcagtgtgatctggccatccaccaccagcagaacatcacgcaggctgacccgctgtttcacccggtcaaggccggtatctgttcgatgaacaagtctcagacctatgcggctgtcgagaagcaggctggcggccctattgagacgctaaaccagcgctaccaggccgaactggcattgatgtcctctgtgttggatttccccaagtccccatattgccagcagcataacatcggcaaactgtgcgacttttcacaggctatgcctagccgcctcaacatctccgatgacgggaatgaggtgcaactcgaaggcgccgtcggtctttcctccacgctcgccgagatcttcctactggaatacgctcagggtatgcctgtggtcgcctggggcaacattcacaacgagagccagtggaagagcctccttaacttgcacaacgcccatttcaacctgatgcacagaacgccctacattgccaagcaccagggaacccctttacttcaggctatcagcaacgctctcaacccaaatgcaactgagtcgaagctccccgatatctctcccgacaacaagatccttttcattgccggccacgacaccaacatcgcaaacatcggaggcatgttgggtatgaactggactctcccgggccagccagacaatactccgcccggcggtggactggttttcgaactctggcagaacccggataaccatcagcagtacgttgcggtgaagatgatctaccagaccatggaccagctgcgcaattccgagaagctggacttgaagagcaaccctgctgggatcgtccccattgagatcgaaggttgcgagaacatcggtaccgacaagctgtgccagctggatacttttcagaagcgtgttgcccaggtcattgagcccgcgtgccaaatctaatagcacgaacctctctgaaggaggttctgagacacgcgcgattcttctgtatatagttttatttttcactctggagtgcttcgctccaccagtacataaacctttttttcacgtaacaaaatggcttcttttcagaccatgtgaaccatcttgatgccttgacctcttcagttctcactttaacgtagttcgcgtttgtctgtatgtcccagatctacgtacaagacgagtgtgccagtcagccatcgaatgatgggtcgtcatggacaacggagcaatgccatctcgggactggggccaccgacctgccggcagggggaggagaaaaaaaagggggcggcgtcggcaagatggcaagaggtctgggagatagtcaaggacgaagggccaagcgggtcgtgtcgcttgcactctggtgaggctcgctagtgcttgcggtgggtgaggtggatcagagtagaggcccgatggagaatgggcgctgcggtgacaagcctaaccggtcgatagcggctagtaggctgggcggcgatgtcacgttatggttgccacggccgggcacggactgagggcatcgttcgaggacgtggagccgtgcgatggaagcgattaaggttgctgaagggtcggcgagggcaaccacacacctcattggaggagtctgggcgcggcagccaggttatcaggggtgacacggaaggcgtccgccgagtgcgactctgcgaagcccaggccgccctgcggaagaaccttggttcacgatgctctctgatgaccgcagaagagcgtatttgacgctccgcgcaaatgcgatccgaagtgccggtgcgaggctcctgtcgcctgctcgtctccgccctgactctcaacgcagcacgaacagcgctgccgtagatactccttcggagcctaccctggtgtacactagattcaagcaaaaggcgcacgagagcggagaaagccgcagcacaccccatcccttcccttcgcaacggatctcgcttctccccgcattttagtgtagtcccggtggcccacttaccccggcttctgcagtcttggtatttggtggtttctgatgtgcggggagagaggccttcagaaaaaaatgtgtctcttcgtggaactcgccgtgtgtgcgactttaacttgtatggaagttgaggtcgatttccccagatcttgaggctcaaaaactcccaaatttggcaccaaacgactgggacgcaaaaccgggtacctggtgccatcttcaatatacttgggaatgcagccgcggtgattgtcggtctgggcgcaacctcatcggcgttggcgggcgatcagcaggtgcaacggggggtgcaaaccgggaaatgagaagagcccagaccaacgaggaccgcggcatcgacctccgatcaccctaagttgggcctagtgccgccccaccaaggaacggaccagggtagtttgctcctcccgatcactggtagtcgtgccacagtgcagcgcattcaagggcaagccgctttcccaaatagagcaggaagggctggtttcaactttgaggtcaggacatccgtaacgttattccactttcggtgttctctttcttccgcagcttcttcggcagcgagttagttgcgcaactctcgtcacttttgcaatactttcatcgacgttccccaaatcggatacctcatcgatgctcttctgcgttaattaacaattgggatcctctagacccgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgctcgatttaaatctcgagaggcctgacgtcgggcccggtaccacgcgtcatatgactagt

SEQ ID NO: 14 - DNA, 9631 bp, synthetic construct
  source 1..9631: mol_type = other DNA
  misc_feature 1..9631: note = pMT2197
gaagaagggcgtaaataagaagctataatagcttaaaggtataattacctaacttagtaagcatactatattagccactatactataaagggctcctagaacgtaactacctatagcttagctaccactagctactataagggctagcgcccctcctagcttaatcgcctacctagtcgtatacctaccgcctacgactactagctagaaacgactacggctagaaacgctagcataggagagcccctcacgtaagagggctactagcgcgctctatagtaaccgtaccctagccagggacacctaggccagagctaggactctcgtacacctacagtaggagcgcccgtcagcactatccgtattcctagacaacctaggcctggctagtagtaagaaggactgacggataagcggatagatacgaccgtaggatagaggaacgattgtaaggacggtcataatagataaaagactatatttactaagttataacgggtcgtcatttttccttttgaaccctggtacctattatagcatttggctatctataggttagagctaactaacttactaatagggataataagctagggtcaatatatatttagatagcttctctaagtgcctctagtatttagaacaattaacgcttttattacctacttctgtagaccgtaacaattagtctaggagtgccgcttatagcgcgaatactgctgtggtatcatacctttacagcctctctaagctgtactgattatagccaccgagctatagcacaaatagctcgcttgacttggactttgatcaactacgatgcgaaaaagaaaagcgagaggaaaatgggaagaaaaaaatttttttaaaaaaaatgaaccatataatttaattagcaccgggattggctgttcgttgcctcttctagcttggttggggagagcggacggaatcccaaaatttccggcacgtatggtcgtttgtaattgggtctaccaaaagttgattagtagtgctcgcctgttgaagttaaggcgcctagaggcatataatattactaataatagtccttacactcttggggtccataaaccgtgtatccctgcgccgatgaatgtgctctccatttgagcaaatagaatagtcgacagcaccacgaattaacaggataaagtacggtatgatgggtcagttcagcaatgtgtgagtagtgtactgtggtaagaaggtaggcggtagagagcgggctctccagaccacagaaagagggctccgtcccacaaaggcccagacccacttccaggggtatttatctcaactacatgcaactgggacatacagacaaacgcgaactacgttaaagtgagaactgaagaggtcaaggcatcaagatggttcacatggtctgaaaagaagccattttgttacgtgaaaaaaaaggtttatgtactggtggagcgaagcactccagagtgaaaaataaaactatatacagaagaatcgcgcgtgtctcagaacctccttcagagaggttcgtcgtaataatgggcgctttctactctggacttgcctctttgtgctttacattgggggtcgagcctttccaatcagaagatgcacttaccaaagtagcgctaggctacacagcgctttacccccgaatcaggcgtggggtcactaccccgacgcccttttttttttttttttttgtattcacagagcggtttgcacaatctagatttgttcctcccgcctagtcggtcgccttgtacttggcccggtactcctccgtcctcctgatgtcctcctcggatgcgagccccttgataccctcgatgatgtcgtccaaggtgagaatcgcaaagatgggcagaccatactccttccggagctcaccgatcgcgctcggcatcggcttgctatcgtcgccatccggcgaaggcagcttctccatgcggtccagcgcgaccacgatgccggctacgataccgccctccttctcaatcttggcgatcgcctcacgcttcgccgtgccagccgtgatcacatcatccacgataagcaccctcttgcccttgagcggcgcccccacaatgttgccgccctcaccgtggtcctttgcctccttgcggtcgaacgagtagcacgtggtatggccgtacgtggccggatccagctggcccagccgaatggtggccgccgtcgccaacgggatccccttgtaggccgggccgaagacgatgtcgaactggatcgcgccgctccggtgcgcctcgatgatggcgtgcgcgtacgccgtcgcgagcgcctcgagcagatcggcccggtagaagtctcccgcgttgaagaagtagggcgagatgcgcttcgacttgagctcgaagctgccaaacttgaggatgttgccggcgatggccgcccggaggaagtcggctttgtaagaagcgagtggggccatgtctgtcggttcgtacgtgtcttacgggttcttgggctggaagtttatttgttaccggagggaactcctggtgaagattcgaaaaaaagaaatgagggagttttgtcaaacgtccccctatcgcagagctgaagagagcccctccatcgcgaatttcttggtggggttggattcatggtggggcccaagggtcgttaaacttttttgtatgtctacggagttacacagtactccgtacgtcgtaccccacttgcaatgcttctcgagctgggcttgggatattttgataacgcacaccgcctgccttatcttatcattttgtgctctccgcaaaaaattatttcgggcgacttggggacggcaaatcgacaccgccccgccaggtgctccctatctgacaactcccaaacacccccatcgccaaattcggacagcaccctgcgcgttgcctgccatggcctggaaggatatcgcgcctgtgcctacgtaagggttcaattcgtatatatgctgttcctttcctgaactaagctgatcattttcgtacagggcacaggagtttatcgacatcatcctttcgtaagtttgaaggctaggttatacgtagcggccgctcgactcccgcgcgccgaggaagaacttcgggtctttgtgacgggagattccactgagtgagcattggccaaccaagcacacaattactccgtacatacacagtacttctgactccgtaaagtaaaccgtgtgtttcaaagatcggtaatccgtaacaggtactccgtatctaaggtaaatttaccctgtgcacggagcagaacctgaacttcttcccccctcttactcgagtagtcaccctactccaaccagcggcttttcaactcgcaaagtcttgtttataacagtgcatatacctgcatttcgtatctcgctagtgtaaagacgaccacacgcggacaaagaaagaaaaatccaattgcccgatggctcttagtttgaggacagcagcgaaggactacactgcgccgtagtgaccaggccaagaaacgcgaatcgtatattaacggcaaatcaaaatggattatatgccatttcgcttccgggttgcgtgctcgtccgaagtctggtgccgatcgattgcgaacccccggaatcgcgggatgattcctacagccgccgaaagggggggggggggaggggggtctggacgggacgtgcataacttcgaatttctagaatattgcggattgggttcccttcagccctgcgagcgcgcccccttctggaaccgcacccttcaccggttccacacacagaggacatgggtggaaatgtgtacctgacggttgcccctttgggacagtggagaggcggatgttcggataaccatccggagccgcagtgtcgaccaagatcttggcttaccatcgacaccaacatgcggactcgtccctcagtcatggagccttggctcgcggagcctccgttcgaagcggctatcccgtcctgccagcggaggatctcgtaccgcttccgcgaactgtgaatgtcctgggtataagagcatggcgcgaccttgtctcgtcaggaacggggaggaggagggcttggttagggtcgcgttcgtttggagattgctgagctctgagccttcggtccttggatccctgcggtccccgctctcctctctctctctctctctctctctctctctctcttcttcccacgctcgttcgacagacgcctccccttcttcgctctcctttccctcgcacgtagcacactaatagtgcaccatggtgactccctctctgaagaaggcagctctggctgccctgtccctcttcccgctcctctccctggctagcgaaaccgaaccttccggatatcagctggagaaggtggtcattctctcgcgtcacggtgtccgagcccccaccaagatgacacagacgatgcgcgatgtcactcctcatcagtggcctgagtggcccgtgaagctcggctacatcactcctcgtggagaacacctcatcagcctgatgggcggtttctatagggaacggttccagcagcagggattgcttcccaacgacacctgtccgacccccgacgccgtctacgtgtggaccgacgttaaccagcgtacccgcaagactggagaggctttcctcgccggtcttgcgcctcagtgtgatctggccatccaccaccagcagaacatcacgcaggctgacccgctgtttcacccggtcaaggccggtatctgttcgatgaacaagtctcagacctatgcggctgtcgagaagcaggctggcggccctattgagacgctaaaccagcgctaccaggccgaactggcattgatgtcctctgtgttggatttccccaagtccccatattgccagcagcataacatcggcaaactgtgcgacttttcacaggctatgcctagccgcctcaacatctccgatgacgggaatgaggtgcaactcgaaggcgccgtcggtctttcctccacgctcgccgagatcttcctactggaatacgctcagggtatgcctgtggtcgcctggggcaacattcacaacgagagccagtggaagagcctccttaacttgcacaacgcccatttcaacctgatgcacagaacgccctacattgccaagcaccagggaacccctttacttcaggctatcagcaacgctctcaacccaaatgcaactgagtcgaagctccccgatatctctcccgacaacaagatccttttcattgccggccacgacaccaacatcgcaaacatcggaggcatgttgggtatgaactggactctcccgggccagccagacaatactccgcccggcggtggactggttttcgaactctggcagaacccggataaccatcagcagtacgttgcggtgaagatgatctaccagaccatggaccagctgcgcaattccgagaagctggacttgaagagcaaccctgctgggatcgtccccattgagatcgaaggttgcgagaacatcggtaccgacaagctgtgccagctggatacttttcagaagcgtgttgcccaggtcattgagcccgcgtgccaaatctaatagcacgaacctctctgaaggaggttctgagacacgcgcgattcttctgtatatagttttatttttcactctggagtgcttcgctccaccagtacataaacctttttttcacgtaacaaaatggcttcttttcagaccatgtgaaccatcttgatgccttgacctcttcagttctcactttaacgtagttcgcgtttgtctgtatgtcccagatctacgtacaagacgagtgtgccagtcagccatcgaatgatgggtcgtcatggacaacggagcaatgccatctcgggactggggccaccgacctgccggcagggggaggagaaaaaaaagggggcggcgtcggcaagatggcaagaggtctgggagatagtcaaggacgaagggccaagcgggtcgtgtcgcttgcactctggtgaggctcgctagtgcttgcggtgggtgaggtggatcagagtagaggcccgatggagaatgggcgctgcggtgacaagcctaaccggtcgatagcggctagtaggctgggcggcgatgtcacgttatggttgccacggccgggcacggactgagggcatcgttcgaggacgtggagccgtgcgatggaagcgattaaggttgctgaagggtcggcgagggcaaccacacacctcattggaggagtctgggcgcggcagccaggttatcaggggtgacacggaaggcgtccgccgagtgcgactctgcgaagcccaggccgccctgcggaagaaccttggttcacgatgctctctgatgaccgcagaagagcgtatttgacgctccgcgcaaatgcgatccgaagtgccggtgcgaggctcctgtcgcctgctcgtctccgccctgactctcaacgcagcacgaacagcgctgccgtagatactccttcggagcctaccctggtgtacactagattcaagcaaaaggcgcacgagagcggagaaagccgcagcacaccccatcccttcccttcgcaacggatctcgcttctccccgcattttagtgtagtcccggtggcccacttaccccggcttctgcagtcttggtatttggtggtttctgatgtgcggggagagaggccttcagaaaaaaatgtgtctcttcgtggaactcgccgtgtgtgcgactttaacttgtatggaagttgaggtcgatttccccagatcttgaggctcaaaaactcccaaatttggcaccaaacgactgggacgcaaaaccgggtacctggtgccatcttcaatatacttgggaatgcagccgcggtgattgtcggtctgggcgcaacctcatcggcgttggcgggcgatcagcaggtgcaacggggggtgcaaaccgggaaatgagaagagcccagaccaacgaggaccgcggcatcgacctccgatcaccctaagttgggcctagtgccgccccaccaaggaacggaccagggtagtttgctcctcccgatcactggtagtcgtgccacagtgcagcgcattcaagggcaagccgctttcccaaatagagcaggaagggctggtttcaactttgaggtcaggacatccgtaacgttattccactttcggtgttctctttcttccgcagcttcttcggcagcgagttagttgcgcaactctcgtcacttttgcaatactttcatcgacgttccccaaatcggatacctcatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgctcgatttaaatctcgagaggcctgacgtcgggcccggtaccacgcgtcatatgactagt

SEQ ID NO: 15 - DNA, 9962 bp, synthetic construct
  source 1..9962: mol_type = other DNA
  misc_feature 1..9962: note = pMT2651
gaagaagggcgtaaataagaagctataatagcttaaaggtataattacctaacttagtaagcatactatattagccactatactataaagggctcctagaacgtaactacctatagcttagctaccactagctactataagggctagcgcccctcctagcttaatcgcctacctagtcgtatacctaccgcctacgactactagctagaaacgactacggctagaaacgctagcataggagagcccctcacgtaagagggctactagcgcgctctatagtaaccgtaccctagccagggacacctaggccagagctaggactctcgtacacctacagtaggagcgcccgtcagcactatccgtattcctagacaacctaggcctggctagtagtaagaaggactgacggataagcggatagatacgaccgtaggatagaggaacgattgtaaggacggtcataatagataaaagactatatttactaagttataacgggtcgtcatttttccttttgaaccctggtacctattatagcatttggctatctataggttagagctaactaacttactaatagggataataagctagggtcaatatatatttagatagcttctctaagtgcctctagtatttagaacaattaacgcttttattacctacttctgtagaccgtaacaattagtctaggagtgccgcttatagcgcgaatactgctgtggtatcatacctttacagcctctctaagctgtactgattatagccaccgagctatagcacaaatagctcgcttgacttggactttgatcaactacgatgcgaaaaagaaaagcgagaggaaaatgggaagaaaaaaatttttttaaaaaaaatgaaccatataatttaattagcaccgggattggctgttcgttgcctcttctagcttggttggggagagcggacggaatcccaaaatttccggcacgtatggtcgtttgtaattgggtctaccaaaagttgattagtagtgctcgcctgttgaagttaaggcgcctagaggcatataatattactaataatagtccttacactcttggggtccataaaccgtgtatccctgcgccgatgaatgtgctctccatttgagcaaatagaatagtcgacagcaccacgaattaacaggataaagtacggtatgatgggtcagttcagcaatgtgtgagtagtgtactgtggtaagaaggtaggcggtagagagcgggctctccagaccacagaaagagggctccgtcccacaaaggcccagacccacttccaggggtatttatctcaactacatgcaactgggacatacagacaaacgcgaactacgttaaagtgagaactgaagaggtcaaggcatcaagatggttcacatggtctgaaaagaagccattttgttacgtgaaaaaaaaggtttatgtactggtggagcgaagcactccagagtgaaaaataaaactatatacagaagaatcgcgcgtgtctcagaacctccttcagagaggttcgtcgtaataatgggcgctttctactctggacttgcctctttgtgctttacattgggggtcgagcctttccaatcagaagatgcacttaccaaagtagcgctaggctacacagcgctttacccccgaatcaggcgtggggtcactaccccgacgcccttttttttttttttttttgtattcacagagcggtttgcacaatctagatttgttcctcccgcctagtcggtcgccttgtacttggcccggtactcctccgtcctcctgatgtcctcctcggatgcgagccccttgataccctcgatgatgtcgtccaaggtgagaatcgcaaagatgggcagaccatactccttccggagctcaccgatcgcgctcggcatcggcttgctatcgtcgccatccggcgaaggcagcttctccatgcggtccagcgcgaccacgatgccggctacgataccgccctccttctcaatcttggcgatcgcctcacgcttcgccgtgccagccgtgatcacatcatccacgataagcaccctcttgcccttgagcggcgcccccacaatgttgccgccctcaccgtggtcctttgcctccttgcggtcgaacgagtagcacgtggtatggccgtacgtggccggatccagctggcccagccgaatggtggccgccgtcgccaacgggatccccttgtaggccgggccgaagacgatgtcgaactggatcgcgccgctccggtgcgcctcgatgatggcgtgcgcgtacgccgtcgcgagcgcctcgagcagatcggcccggtagaagtctcccgcgttgaagaagtagggcgagatgcgcttcgacttgagctcgaagctgccaaacttgaggatgttgccggcgatggccgcccggaggaagtcggctttgtaagaagcgagtggggccatgtctgtcggttcgtacgtgtcttacgggttcttgggctggaagtttatttgttaccggagggaactcctggtgaagattcgaaaaaaagaaatgagggagttttgtcaaacgtccccctatcgcagagctgaagagagcccctccatcgcgaatttcttggtggggttggattcatggtggggcccaagggtcgttaaacttttttgtatgtctacggagttacacagtactccgtacgtcgtaccccacttgcaatgcttctcgagctgggcttgggatattttgataacgcacaccgcctgccttatcttatcattttgtgctctccgcaaaaaattatttcgggcgacttggggacggcaaatcgacaccgccccgccaggtgctccctatctgacaactcccaaacacccccatcgccaaattcggacagcaccctgcgcgttgcctgccatggcctggaaggatatcgcgcctgtgcctacgtaagggttcaattcgtatatatgctgttcctttcctgaactaagctgatcattttcgtacagggcacaggagtttatcgacatcatcctttcgtaagtttgaaggctaggttatacgtagcggccgctcgattgggcgggaggaagcgttgcaacacgcactatcgccaaggtggactcggcgcaatctggaggttcggcccgcggaggacggaatccgggctgaatctgcgcaaaggctgaccctgcgatggtgggaaaatgtaaatatgtgaagttataggcatataggactcagcgatgacatggaaattgcagaggcatgtgggatttcagcgtttggcatgcattggtcggatctctcgccttgtctgatgtgatcccgccggaggtgtttcgctctctggggaagggaccccccctggccccccacctgccccgcatcatgcctcgccacgactcccgcgcgccgaggaagaacttcgggtctttgtgacgggagattccactgagtgagcattggccaaccaagcacacaattactccgtacatacacagtacttctgactccgtaaagtaaaccgtgtgtttcaaagatcggtaatccgtaacaggtactccgtatctaaggtaaatttaccctgtgcacggagcagaacctgaacttcttcccccctcttactcgagtagtcaccctactccaaccagcggcttttcaactcgcaaagtcttgtttataacagtgcatatacctgcatttcgtatctcgctagtgtaaagacgaccacacgcggacaaagaaagaaaaatccaattgcccgatggctcttagtttgaggacagcagcgaaggactacactgcgccgtagtgaccaggccaagaaacgcgaatcgtatattaacggcaaatcaaaatggattatatgccatttcgcttccgggttgcgtgctcgtccgaagtctggtgccgatcgattgcgaacccccggaatcgcgggatgattcctacagccgccgaaagggggggggggggaggggggtctggacgggacgtgcataacttcgaatttctagaatattgcggattgggttcccttcagccctgcgagcgcgcccccttctggaaccgcacccttcaccggttccacacacagaggacatgggtggaaatgtgtacctgacggttgcccctttgggacagtggagaggcggatgttcggataaccatccggagccgcagtgtcgaccaagatcttggcttaccatcgacaccaacatgcggactcgtccctcagtcatggagccttggctcgcggagcctccgttcgaagcggctatcccgtcctgccagcggaggatctcgtaccgcttccgcgaactgtgaatgtcctgggtataagagcatggcgcgaccttgtctcgtcaggaacggggaggaggagggcttggttagggtcgcgttcgtttggagattgctgagctctgagccttcggtccttggatccctgcggtccccgctctcctctctctctctctctctctctctctctctctcttcttcccacgctcgttcgacagacgcctccccttcttcgctctcctttccctcgcacgtagcacactaatagtgcaccatggtgactccctctctgaagaaggcagctctggctgccctgtccctcttcccgctcctctccctggctagcgaaaccgaaccttccggatatcagctggagaaggtggtcattctctcgcgtcacggtgtccgagcccccaccaagatgacacagacgatgcgcgatgtcactcctcatcagtggcctgagtggcccgtgaagctcggctacatcactcctcgtggagaacacctcatcagcctgatgggcggtttctatagggaacggttccagcagcagggattgcttcccaacgacacctgtccgacccccgacgccgtctacgtgtggaccgacgttaaccagcgtacccgcaagactggagaggctttcctcgccggtcttgcgcctcagtgtgatctggccatccaccaccagcagaacatcacgcaggctgacccgctgtttcacccggtcaaggccggtatctgttcgatgaacaagtctcagacctatgcggctgtcgagaagcaggctggcggccctattgagacgctaaaccagcgctaccaggccgaactggcattgatgtcctctgtgttggatttccccaagtccccatattgccagcagcataacatcggcaaactgtgcgacttttcacaggctatgcctagccgcctcaacatctccgatgacgggaatgaggtgcaactcgaaggcgccgtcggtctttcctccacgctcgccgagatcttcctactggaatacgctcagggtatgcctgtggtcgcctggggcaacattcacaacgagagccagtggaagagcctccttaacttgcacaacgcccatttcaacctgatgcacagaacgccctacattgccaagcaccagggaacccctttacttcaggctatcagcaacgctctcaacccaaatgcaactgagtcgaagctccccgatatctctcccgacaacaagatccttttcattgccggccacgacaccaacatcgcaaacatcggaggcatgttgggtatgaactggactctcccgggccagccagacaatactccgcccggcggtggactggttttcgaactctggcagaacccggataaccatcagcagtacgttgcggtgaagatgatctaccagaccatggaccagctgcgcaattccgagaagctggacttgaagagcaaccctgctgggatcgtccccattgagatcgaaggttgcgagaacatcggtaccgacaagctgtgccagctggatacttttcagaagcgtgttgcccaggtcattgagcccgcgtgccaaatctaatagcacgaacctctctgaaggaggttctgagacacgcgcgattcttctgtatatagttttatttttcactctggagtgcttcgctccaccagtacataaacctttttttcacgtaacaaaatggcttcttttcagaccatgtgaaccatcttgatgccttgacctcttcagttctcactttaacgtagttcgcgtttgtctgtatgtcccagatctacgtacaagacgagtgtgccagtcagccatcgaatgatgggtcgtcatggacaacggagcaatgccatctcgggactggggccaccgacctgccggcagggggaggagaaaaaaaagggggcggcgtcggcaagatggcaagaggtctgggagatagtcaaggacgaagggccaagcgggtcgtgtcgcttgcactctggtgaggctcgctagtgcttgcggtgggtgaggtggatcagagtagaggcccgatggagaatgggcgctgcggtgacaagcctaaccggtcgatagcggctagtaggctgggcggcgatgtcacgttatggttgccacggccgggcacggactgagggcatcgttcgaggacgtggagccgtgcgatggaagcgattaaggttgctgaagggtcggcgagggcaaccacacacctcattggaggagtctgggcgcggcagccaggttatcaggggtgacacggaaggcgtccgccgagtgcgactctgcgaagcccaggccgccctgcggaagaaccttggttcacgatgctctctgatgaccgcagaagagcgtatttgacgctccgcgcaaatgcgatccgaagtgccggtgcgaggctcctgtcgcctgctcgtctccgccctgactctcaacgcagcacgaacagcgctgccgtagatactccttcggagcctaccctggtgtacactagattcaagcaaaaggcgcacgagagcggagaaagccgcagcacaccccatcccttcccttcgcaacggatctcgcttctccccgcattttagtgtagtcccggtggcccacttaccccggcttctgcagtcttggtatttggtggtttctgatgtgcggggagagaggccttcagaaaaaaatgtgtctcttcgtggaactcgccgtgtgtgcgactttaacttgtatggaagttgaggtcgatttccccagatcttgaggctcaaaaactcccaaatttggcaccaaacgactgggacgcaaaaccgggtacctggtgccatcttcaatatacttgggaatgcagccgcggtgattgtcggtctgggcgcaacctcatcggcgttggcgggcgatcagcaggtgcaacggggggtgcaaaccgggaaatgagaagagcccagaccaacgaggaccgcggcatcgacctccgatcaccctaagttgggcctagtgccgccccaccaaggaacggaccagggtagtttgctcctcccgatcactggtagtcgtgccacagtgcagcgcattcaagggcaagccgctttcccaaatagagcaggaagggctggtttcaactttgaggtcaggacatccgtaacgttattccactttcggtgttctctttcttccgcagcttcttcggcagcgagttagttgcgcaactctcgtcacttttgcaatactttcatcgacgttccccaaatcggatacctcatcgatgctcttctgcgttaattaacaattgggatcctctagacccgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgctcgatttaaatctcgagaggcctgacgtcgggcccggtaccacgcgtcatatgactagt

SEQ ID NO: 16 - DNA, 1630 bp, Thermothelomyces thermophilus
  source 1..1630: mol_type = genomic DNA
  misc_feature 1..1630: note = cbh3 Promoter
cattcggattttcccttaattatatgaagtattcggcccacggaaccgggccccgagcaggttgaaccgcgcaaaacctcaaccgagtcacctcgcgtccatgtttgtcatggaatcaggctccgaatcccgtcagatcagtcagttctggtggctatggacgcgggagttacggccagtcgtcccgttgttctggggggttgatcaacaggaggaagagatctgagatcgaactacacccattgatttatcgacgcataatcaagtttaataaaaaccaaacagcgtgtttggtgctaccaccgaatgcgagatccgggctagcccgcggaaggatgatggccacagatctagcgtcatgtatgattattacctatgcatctatcttcgtatctgcctcgggttggcaacacctgaccgagagacgactcgacaacctgacacttggcaaaagacatttcggttgacagcgggagaactccagcgaggaagtcgcccagagatgcggatgagaagacaacgccgagacgtgccggcgttggctctccacgaatcggagccgactcttccgtttggccaatctccgggataaatcccagcggcgggtcacgtcacgtttcatggggaggcgcggacagccatcccagccaggccatggaagagaacaattcttgggggtagcgaccgagccaaaaggggggggggggaagcgggaggggaagaagtggtattagagcacgcaccggaaaacgcatttgggcccttgccaacaaacaccacaccccgcgtcctgggagcaagacatccaggatgcaacccagtaggggatgccaagaagcatctacggcaccatctgccggcgcctcgcctgttagagtcccggcacccgccaatggggccgtgctgggccctgcccggcaatgctggcgcagcggcatcaacaacattgctcggggaggggcccgattttattgattagcaaaaaaacaattaaattacccttccattccagcagagcttctcctccacgcggcggcgggaccgcttgtggacggcggtacactacaaccgcggggctccagtctccgtgctgggcgtgcagatcacgacccggaagagaaatgatcgcggtctgacgccgggtacggagtactgagccgccaaccacagccgatggaccgtgatatctcaatgcgttcaagcaacacagcaacaccctggacgagtctctcctcccctaccaccccctccccccctgccctggccgcgaacggggcgcgtaccccagatttctactccgtactgacaccccaatctattcccgctggcgtcgcccagtctggggcggtccggccaagactctcggtgcacgataccgcgacgaaatcggattaaccgttggctgatcaattccaagtcaagggagaagtggtatggaaagtcggctcagttttccactgcccccgacaggcaggttccggatctggacagcagtcttccgaatctttggcagagactcatgataatataaaaaggcaaatgaggcggcgccttggacaggtccattctcccaccgctcaaccagcctccaattcctcagaagtctgttgctctctcgcagtcgcagtca

SEQ ID NO: 17 - DNA, 2149 bp, Thermothelomyces thermophilus
  source 1..2149: mol_type = genomic DNA
  misc_feature 1..2149: note = cbh3 Promoter
ccaatagcagagtgcggagaagtctgagagagactctggcagcggagtcccttgcactttaattacttgatactccatataccgtttcggccactcagattctaggggtagggcgggagcagaggcgaaaattgggttgtagaatatgaggagctagggttgttaaactcaaagaacttcttgctcttgttcttagtcttctctcctgggaaaagggggtttttccgaaagcggcgctatacgaagccagaggctactttccttgctttggatggcccttgtccaccgttcttgtttcccgtttgtcaattgcgacgttgccggcaacctaggtcctaataattaggtagatatttcggtagaggtagtttaattatgcttcagtagagaaatcgttgtctccacgtctcgcaaccttgcgaaacttcgccacattgaagatagcattgtctgagttgattttaaccctttccagagacgatataatagtgcaagtttctttgatcggaatcatcgacattcggattttcccttaattatatgaagtattcggcccacggaaccgggccccgagcaggttgaaccgcgcaaaacctcaaccgagtcacctcgcgtccatgtttgtcatggaatcaggctccgaatcccgtcagatcagtcagttctggtggctatggacgcgggagttacggccagtcgtcccgttgttctggggggttgatcaacaggaggaagagatctgagatcgaactacacccattgatttatcgacgcataatcaagtttaataaaaaccaaacagcgtgtttggtgctaccaccgaatgcgagatccgggctagcccgcggaaggatgatggccacagatctagcgtcatgtatgattattacctatgcatctatcttcgtatctgcctcgggttggcaacacctgaccgagagacgactcgacaacctgacacttggcaaaagacatttcggttgacagcgggagaactccagcgaggaagtcgcccagagatgcggatgagaagacaacgccgagacgtgccggcgttggctctccacgaatcggagccgactcttccgtttggccaatctccgggataaatcccagcggcgggtcacgtcacgtttcatggggaggcgcggacagccatcccagccaggccatggaagagaacaattcttgggggtagcgaccgagccaaaaggggggggggggaagcgggaggggaagaagtggtattagagcacgcaccggaaaacgcatttgggcccttgccaacaaacaccacaccccgcgtcctgggagcaagacatccaggatgcaacccagtaggggatgccaagaagcatctacggcaccatctgccggcgcctcgcctgttagagtcccggcacccgccaatggggccgtgctgggccctgcccggcaatgctggcgcagcggcatcaacaacattgctcggggaggggcccgattttattgattagcaaaaaaacaattaaattacccttccattccagcagagcttctcctccacgcggcggcgggaccgcttgtggacggcggtacactacaaccgcggggctccagtctccgtgctgggcgtgcagatcacgacccggaagagaaatgatcgcggtctgacgccgggtacggagtactgagccgccaaccacagccgatggaccgtgatatctcaatgcgttcaagcaacacagcaacaccctggacgagtctctcctcccctaccaccccctccccccctgccctggccgcgaacggggcgcgtaccccagatttctactccgtactgacaccccaatctattcccgctggcgtcgcccagtctggggcggtccggccaagactctcggtgcacgataccgcgacgaaatcggattaaccgttggctgatcaattccaagtcaagggagaagtggtatggaaagtcggctcagttttccactgcccccgacaggcaggttccggatctggacagcagtcttccgaatctttggcagagactcatgataatataaaaaggcaaatgaggcggcgccttggacaggtccattctcccaccgctcaaccagcctccaattcctcagaagtctgttgctctctcgcagtcgcagtcaag

SEQ ID NO: 18 - DNA, 1178 bp, Thermothelomyces thermophilus
  source 1..1178: mol_type = genomic DNA
  misc_feature 1..1178: note = egl5 Promoter
ggatacgagctctctcggggacgaatagatgaaggcaatctgacagatttgctctcaaaaaaagactgatatctcttccaccatgcactgtatgtacattacatacattatccccctccactggattcgcacaacggaaagcaatggcgcgctgattcaagaaccatcagggctgtcattggcttgttttgtgccgtggccgcggtgacgcccactatgactctctgggcaggcggcaactgggtgccagatatattaatccggggcatagcgcatatcttccttgatttgtagagtactagtacactaacccccttctccacatggggccactgttcggtagatctgcccgaagtgcaagtgcggggggggccaaactaggtaatatcctcccgctctcccgagtgcgcggactaaccgtcattgctcccagaggcttgcactctatcgcaggccttttccaataaggatggggcgttcggcggtgatgatgccggtcgtgcggggcatacggggagggtagatagaaaataacgacgctggtgttttggagaggggagggggactattaggggagggaaatacaggggcagggggtgagacgggtgacgttccggcggaacctcgcgcttgtcaaacaagcagccctgttaggttgctctagactagtgtacatacatacatatgtacatactgtatgtactgcacatactttaacttggtgcttccctgtgagccgccaggaacatcacaactgcaagcggaaaaggccccatatacggggcggcttgtcgggatggctccccccttcggaacgggtctgacttccgaggattttacctgcttcatttgggtattctgcgatggcctgttcaacccttcccctggccgaaccgtttcttggctcgatcctagtgtacactacactactcgtagactgcctgcccgacgatccgcgggaacgggccaggagtgtggagtggagacgggcggcggtgatgtcgtgtaattaaatatataagtgagagtgttttttgactgccccgggttctggtagttgaagggaagttcgatgctctctgctgtcgtcgctctcgtcgctctcgtcggcatcctccatccgtccgcctttgataacccgctccccgactcagtcaagacgacgcatacttggcacc

SEQ ID NO: 19 - DNA, 1295 bp, Thermothelomyces thermophilus
  source 1..1295: mol_type = genomic DNA
  misc_feature 1..1295: note = egl5 Promoter
gtctcttgttgtgaaccttgctgttgcatgggcaggaccggtgcatcatgaacaggacggtgcgctgtgttttttttttctcggggtcttgattgtttgttgaatctcccttttcgaggatacgagctctctcggggacgaatagatgaaggcaatctgacagatttgctctcaaaaaaagactgatatctcttccaccatgcactgtatgtacattacatacattatccccctccactggattcgcacaacggaaagcaatggcgcgctgattcaagaaccatcagggctgtcattggcttgttttgtgccgtggccgcggtgacgcccactatgactctctgggcaggcggcaactgggtgccagatatattaatccggggcatagcgcatatcttccttgatttgtagagtactagtacactaacccccttctccacatggggccactgttcggtagatctgcccgaagtgcaagtgcggggggggccaaactaggtaatatcctcccgctctcccgagtgcgcggactaaccgtcattgctcccagaggcttgcactctatcgcaggccttttccaataaggatggggcgttcggcggtgatgatgccggtcgtgcggggcatacggggagggtagatagaaaataacgacgctggtgttttggagaggggagggggactattaggggagggaaatacaggggcagggggtgagacgggtgacgttccggcggaacctcgcgcttgtcaaacaagcagccctgttaggttgctctagactagtgtacatacatacatatgtacatactgtatgtactgcacatactttaacttggtgcttccctgtgagccgccaggaacatcacaactgcaagcggaaaaggccccatatacggggcggcttgtcgggatggctccccccttcggaacgggtctgacttccgaggattttacctgcttcatttgggtattctgcgatggcctgttcaacccttcccctggccgaaccgtttcttggctcgatcctagtgtacactacactactcgtagactgcctgcccgacgatccgcgggaacgggccaggagtgtggagtggagacgggcggcggtgatgtcgtgtaattaaatatataagtgagagtgttttttgactgccccgggttctggtagttgaagggaagttcgatgctctctgctgtcgtcgctctcgtcgctctcgtcggcatcctccatccgtccgcctttgataacccgctccccgactcagtcaagacgacgcatacttggcacc

SEQ ID NO: 20 - DNA, 1574 bp, Thermothelomyces thermophilus
  source 1..1574: mol_type = genomic DNA
  misc_feature 1..1574: note = cbh2 Promoter
cgatgcgtctcgtggaaagttaggcacctcccccctttcggggcgagtagccgaagcgggggcatcaactgattaacaggcttgttaaaggaagtcttcacggtcggcatacaaatcggccaagacgttgaagatgccataatacataatatgtatgtacaataaccgggcacatacggtgcaactgctagtcagcaattgcgcctcgctttttgacacagagggcaatgcagaacgtcgaatcgaccgagtcagttgctgttggcgtcatgatggctctagggtcagacagaaggtcagagaaagcatgccttagctcgaagccgctcagatgtaattacgcaacgctcggcgttcgagtttacggaggacgacggctacaagatggggctgcttaaagttaccttaaatagaaaatagtgcctggcttaagagatcatgtccgcggggctagcaaggatgtcgggtcttaactcgacggctcgcctagatttcgtgaaaagggaactcactccccgacaggcccgcaagtgaatatgtaattactcaatggaagttctcgaaacggagtccagaaatgatgtggttctgtgggaatgcggcaagaggcgacgttgccgtgaatgcgtgaacattcccgcctcttcttcttctcgtcttcttccttcttcttctttcgggtcgcggatggttgacggccagcgtgcgcacggctgcgtgttatcgagcgtcggtacgtctagccaacatcccgtagacacgacgaccaagcgtcttgagaatgcaacaacgtctcggaacctggcacgcatcttccgccgcaggtcggcagacgccgcctgggcaataccacccctgtccaggccctttccccgcaggcagagccgcgctcttcctttcatggttattcaggaacgtggcttccgagattctcgcctgttctcccccagtcaacctgccgaccgtaacccggttccaccaccgcggactgtccgcaaaacctggttcgcccgagattaatatgctatttccggactaagtgcacaacacacaagcaccccttccgcctcgcgctctagaatctgctttctaacccggttctcgggcccttccctttcgcgacgcctccgctctccttaccaggcaccatccgcaataggtaaggtagccaaccgttttggagcgtgattctgccaaggaccgcatccttgcattcgccatctggtcaaggacccctctttcccgctccattctggtggctctatcgggacggcgttccccatggctctccaggagagtgatgtgcgagtctggagagccggggttggcgtcacgatgctgcccacctagggccggccagcccggcactgcgctcccgttgatccgtctatccccgtcaagagcaccagccccggcgctcgtgaattttcgacttgttcgacttgctacaggtgataaagaggatgcacgccgccctcgatcggcctgtgtggtttctctccctcgtgccaaaccactcccacctcccgccccgagatagttgcttgtttcgctccgtgagagggacacacacc

SEQ ID NO: 21 - DNA, 2434 bp, Thermothelomyces thermophilus
  source 1..2434: mol_type = genomic DNA
  misc_feature 1..2434: note = cbh2 Promoter
tgtgaaatcctcgggcattaggcactagagtttagcgaaaaggtttttacagtactcggtagttcgtcgcctttttcgtgccgctgacggggagcgtttccaagttttggtgaccaaatcagtgactgaacgccaagaaactcctgtcaaccaagcaggcgcaccgctgaacccgcccacgtgggtccgacccccgttcactgctttcgatgaggtggttccatggaccaaggaaacacttgatggcgctcgtcaccagaacgacgaaggcttctcgacttcgatctcaatgtcgttccctatatgtactgtactgatacatacataccacgtaaaggtgccccttagcacgtcctgcagcaatcctcagagctccgctctcatcagatgcataccgcaattgataagtggctacggagtagttacgggcgacatatcaagcgtagcgaccttgattcccagtaccgagcctatacctattgaaataatctgcattatcccatcacagatcctccttagccaggcttacctggggccgcttctggagcttcacattcccatccaacccgctgccgctgtaaactgcgaatgggttcattgtattttttttttgttgttgttttgaatagaaaactctgattccgtatcacccattcatacaaaccactagtcccttattcgaggcgaccggctcctctctgcagaagatgcctcctctctcccacagtcacgggccagagaactcgaaaattctcgcagtacaagatccccctagagattagtctaatttaactcgaatcgcgaccataaccgagttgatggatattcaccctacacgcccaatgctcgagcttgctcgacgatgcgtctcgtggaaagttaggcacctcccccctttcggggcgagtagccgaagcgggggcatcaactgattaacaggcttgttaaaggaagtcttcacggtcggcatacaaatcggccaagacgttgaagatgccataatacataatatgtatgtacaataaccgggcacatacggtgcaactgctagtcagcaattgcgcctcgctttttgacacagagggcaatgcagaacgtcgaatcgaccgagtcagttgctgttggcgtcatgatggctctagggtcagacagaaggtcagagaaagcatgccttagctcgaagccgctcagatgtaattacgcaacgctcggcgttcgagtttacggaggacgacggctacaagatggggctgcttaaagttaccttaaatagaaaatagtgcctggcttaagagatcatgtccgcggggctagcaaggatgtcgggtcttaactcgacggctcgcctagatttcgtgaaaagggaactcactccccgacaggcccgcaagtgaatatgtaattactcaatggaagttctcgaaacggagtccagaaatgatgtggttctgtgggaatgcggcaagaggcgacgttgccgtgaatgcgtgaacattcccgcctcttcttcttctcgtcttcttccttcttcttctttcgggtcgcggatggttgacggccagcgtgcgcacggctgcgtgttatcgagcgtcggtacgtctagccaacatcccgtagacacgacgaccaagcgtcttgagaatgcaacaacgtctcggaacctggcacgcatcttccgccgcaggtcggcagacgccgcctgggcaataccacccctgtccaggccctttccccgcaggcagagccgcgctcttcctttcatggttattcaggaacgtggcttccgagattctcgcctgttctcccccagtcaacctgccgaccgtaacccggttccaccaccgcggactgtccgcaaaacctggttcgcccgagattaatatgctatttccggactaagtgcacaacacacaagcaccccttccgcctcgcgctctagaatctgctttctaacccggttctcgggcccttccctttcgcgacgcctccgctctccttaccaggcaccatccgcaataggtaaggtagccaaccgttttggagcgtgattctgccaaggaccgcatccttgcattcgccatctggtcaaggacccctctttcccgctccattctggtggctctatcgggacggcgttccccatggctctccaggagagtgatgtgcgagtctggagagccggggttggcgtcacgatgctgcccacctagggccggccagcccggcactgcgctcccgttgatccgtctatccccgtcaagagcaccagccccggcgctcgtgaattttcgacttgttcgacttgctacaggtgataaagaggatgcacgccgccctcgatcggcctgtgtggtttctctccctcgtgccaaaccactcccacctcccgccccgagatagttgcttgtttcgctccgtgagagggacacacacc

SEQ ID NO: 22 - DNA, 1485 bp, Thermothelomyces thermophilus
  source 1..1485: mol_type = genomic DNA
  misc_feature 1..1485: note = cbh1 Promoter
tgccgcggtcctcgttggtctgggctcttctcatttcccggtttgcaccccccgttgcacctgctgatcgcccgccaacgccgatgaggttgcgcccagaccgacaatcaccgcggctgcattcccaagtatattgaagatggcaccaggtacccggttttgcgtcccagtcgtttggtgccaaatttgggagtttttgagcctcaagatctggggaaatcgacctcaacttccatacaagttaaagtcgcacacacggcgagttccacgaagagacacatttttttctgaaggcctctctccccgcacatcagaaaccaccaaataccaagactgcagaagccggggtaagtgggccaccgggactacactaaaatgcggggagaagcgagatccgttgcgaagggaagggatggggtgtgctgcggctttctccgctctcgtgcgccttttgcttgaatctagtgtacaccagggtaggctccgaaggagtatctacggcagcgctgttcgtgctgcgttgagagtcagggcggagacgagcaggcgacaggagcctcgcaccggcacttcggatcgcatttgcgcggagcgtcaaatacgctcttctgcggtcatcagagagcatcgtgaaccaaggttcttccgcagggcggcctgggcttcgcagagtcgcactcggcggacgccttccgtgtcacccctgataacctggctgccgcgcccagactcctccaatgaggtgtgtggttgccctcgccgacccttcagcaaccttaatcgcttccatcgcacggctccacgtcctcgaacgatgccctcagtccgtgcccggccgtggcaaccataacgtgacatcgccgcccagcctactagccgctatcgaccggttaggcttgtcaccgcagcgcccattctccatcgggcctctactctgatccacctcacccaccgcaagcactagcgagcctcaccagagtgcaagcgacacgacccgcttggcccttcgtccttgactatctcccagacctcttgccatcttgccgacgccgcccccttttttttctcctccccctgccggcaggtcggtggccccagtcccgagatggcattgctccgttgtccatgacgacccatcattcgatggctgactggcacactcgtcttgtttgagcatcgacggcccgcggcccgtctcccacggtacggaacctcgttgtacagtacctctcgtaatgatacccaacaccggggccgagcgctgggagggcggcgttcccgagaagccgggaaggcggctggccggctgacctttgtgacttggcgatggatgcggccatggagaatgtccgtccgaagcgacgcgacaattagcctggctaccatcgatataaattgggtgattcccagctcttgatgggcgtgtcttctgcctggcagccctcgtcttcagatcaagcaactgtgtgctgatcctcttccgcc

SEQ ID NO: 23 - DNA, 2008 bp, Thermothelomyces thermophilus
  source 1..2008: mol_type = genomic DNA
  misc_feature 1..2008: note = cbh1 Promoter
acctcacgggcagaagctggaggcgcccgggagagagtggatcgagttgcatgcgcaagcgcccttcatctcttcagccgagaatctgttgcctaattacatagtagatgctgggaacagtctgagaagcctgcgcaggttcttcattaattaccaagaacgcgggagaggtgggaagacgccgaaaactcgacgcggaccattcgctgttttccgatcttacaaaaaaaaggtatccgatttggggaacgtcgatgaaagtattgcaaaagtgacgagagttgcgcaactaactcgctgccgaagaagctgcggaagaaagagaacaccgaaagtggaataacgttacggatgtcctgacctcaaagttgaaaccagcccttcctgctctatttgggaaagcggcttgcccttgaatgcgctgcactgtggcacgactaccagtgatcgggaggagcaaactaccctggtccgttccttggtggggcggcactaggcccaacttagggtgatcggaggtcgatgccgcggtcctcgttggtctgggctcttctcatttcccggtttgcaccccccgttgcacctgctgatcgcccgccaacgccgatgaggttgcgcccagaccgacaatcaccgcggctgcattcccaagtatattgaagatggcaccaggtacccggttttgcgtcccagtcgtttggtgccaaatttgggagtttttgagcctcaagatctggggaaatcgacctcaacttccatacaagttaaagtcgcacacacggcgagttccacgaagagacacatttttttctgaaggcctctctccccgcacatcagaaaccaccaaataccaagactgcagaagccggggtaagtgggccaccgggactacactaaaatgcggggagaagcgagatccgttgcgaagggaagggatggggtgtgctgcggctttctccgctctcgtgcgccttttgcttgaatctagtgtacaccagggtaggctccgaaggagtatctacggcagcgctgttcgtgctgcgttgagagtcagggcggagacgagcaggcgacaggagcctcgcaccggcacttcggatcgcatttgcgcggagcgtcaaatacgctcttctgcggtcatcagagagcatcgtgaaccaaggttcttccgcagggcggcctgggcttcgcagagtcgcactcggcggacgccttccgtgtcacccctgataacctggctgccgcgcccagactcctccaatgaggtgtgtggttgccctcgccgacccttcagcaaccttaatcgcttccatcgcacggctccacgtcctcgaacgatgccctcagtccgtgcccggccgtggcaaccataacgtgacatcgccgcccagcctactagccgctatcgaccggttaggcttgtcaccgcagcgcccattctccatcgggcctctactctgatccacctcacccaccgcaagcactagcgagcctcaccagagtgcaagcgacacgacccgcttggcccttcgtccttgactatctcccagacctcttgccatcttgccgacgccgcccccttttttttctcctccccctgccggcaggtcggtggccccagtcccgagatggcattgctccgttgtccatgacgacccatcattcgatggctgactggcacactcgtcttgtttgagcatcgacggcccgcggcccgtctcccacggtacggaacctcgttgtacagtacctctcgtaatgatacccaacaccggggccgagcgctgggagggcggcgttcccgagaagccgggaaggcggctggccggctgacctttgtgacttggcgatggatgcggccatggagaatgtccgtccgaagcgacgcgacaattagcctggctaccatcgatataaattgggtgattcccagctcttgatgggcgtgtcttctgcctggcagccctcgtcttcagatcaagcaactgtgtgctgatcctcttccgcc

(19) United States
(12) Patent Application Publication
(10) Pub. No.: US 2026/0209782 A1
(43) Pub. Date: Jul. 23, 2026

(54) NEW CELLULASE PROMOTERS FOR FUNGAL PROTEIN PRODUCTION

(71) Applicant: BASF SE, Ludwigshafen (DE)

(72) Inventors: Katja Gemperlein, Ludwigshafen (DE); Stefan Haefner, Ludwigshafen (DE); Sebastian Christopher Spohner, Ludwigshafen (DE)

(21) Appl. No.: 19/139,907

(22) PCT Filed: Dec. 15, 2023
(86) PCT No.: PCT/EP2023/086105

§ 371 Date: Jun. 17, 2025

(30) Foreign Application Priority Data
Dec. 19, 2022 (EP) 22214739.9

(51) Int. Cl. C12N 15/80; C12N 9/16

(52) U.S. Cl. CPC C12N 15/80; C12N 9/16

(57) ABSTRACT

The present invention relates to novel promoters for robust and strong expression in filamentous fungi.

The present invention relates to novel promoters for strong expression in filamentous fungi.

### INTRODUCTION

Biocatalytical and fermentative production of recombinant proteins and fine chemicals has been applied in industrial scale for a long time. In recent years, demand for products derived from biological processes is increasing. Various organisms have been used in such production including bacteria and fungi. Especially filamentous fungi, although difficult to transform and modify genetically have been used more and more as they have been proven to be highly efficient in biocatalytical and fermentative production.

Filamentous fungi have been shown to be excellent hosts for the production of a variety of proteins. Fungal strains such as Aspergillus, Trichoderma, Penicillium and Thermothelomyces have been applied in the industrial production of a wide range of enzymes, since they can secrete large amounts of protein into the fermentation broth. The protein-secreting capacity of these fungi makes them preferred hosts for the targeted production of specific enzymes or enzyme mixtures.

Wild type Thermothelomyces thermophilus (T. thermophilus) C1 (also known as Myceliophthora thermophila, previously described as Chrysosporium lucknowense) is a thermotolerant ascomycetous filamentous fungus which has been described as being attractive for production of cellulases and other proteins on a commercial scale. Only recently, the strain has also been shown to be highly efficient for producing various fine chemicals, e.g. in WO20/161682 it is disclosed that T. thermophilus C1 is capable of producing cannabinoids and precursors thereof. WO00/20555 and US2012/0005812 disclose transformation systems for T. thermophilus C1 and describe expressing and secreting heterologous proteins or polypeptides. Also disclosed is a process for producing large amounts of polypeptides or proteins in an economical manner. WO15/004241 discloses multiple proteases deficient filamentous fungal cells and methods useful for the production of heterologous proteins.

However, for strong expression of recombinant proteins in filamentous fungi, especially in T. thermophilus C1, only a limited number of suitable promoters are known in the art (e.g. Visser et al 2011, Industrial Biotechnology 7 (3), US2014/127788). There is a need for further regulatory elements functional in T. thermophilus C1 that allow robust and strong expression systems of recombinant proteins.

### DETAILED DESCRIPTION OF THE INVENTION

Surprisingly we found that promoters of the cbh3 gene and the cbh2 gene derived from Thermothelomyces thermophilus having the sequence of SEQ ID NO: 16, 17, 20 or 21 and fragments and derivatives thereof are capable to direct strong and robust expression in filamentous fungi of the phylum Ascomycota, especially T. thermophilus C1.

A first embodiment of the invention is an isolated promoter capable of conferring strong and reliable expression in a microorganism of the phylum Ascomycota, preferably Acremonium, Aspergillus, Agaricus, Aureobasidium, Cryptococcus, Corynascus, Chrysosporium, Filibasidium, Fusarium, Humicola, Magnaporthe, Monascus, Mucor, Myceliophthora, Mortierella, Neocallimastix, Neurospora, Paecilomyces, Penicillium, Piromyces, Phanerochaete, Podospora, Pycnoporus, Rhizopus, Schizophyllum, Sordaria, Talaromyces, Rasamsonia, Thermoascus, Thermothelomyces, Thielavia, Tolypocladium, Trametes and Trichoderma, more preferred Aspergillus niger, Aspergillus oryzae, Aspergillus fumigatus, Neurospora crassa, Penicillium chrysogenum, Penicillium citrinum, Acremonium chrysogenum, Trichoderma reesei, Rasamsonia emersonii (formerly known as Talaromyces emersonii), Aspergillus sojae, Thermothelomyces heterothallica and Thermothelomyces thermophilus (formerly known as Myceliophthora thermophila and as Chrysosporium lucknowense), most preferably Thermothelomyces thermophilus, comprising a sequence selected from the group consisting of:
(i) a sequence set forth in SEQ ID NO: 16, 17, 20 or 21,
(ii) a sequence having at least 70% sequence identity for example at least 71%, 72%, 73%, 74%, 75%, 76%, 77%, 78%, 79%, preferably at least 80%, for example 81%, 82%, 83%, 84%, 85%, 86%, 87%, 88% or 89%, more preferably at least 90%, for example at least 91%, 92%, 93%, 94%, 95%, 96% or 97%, even more preferably at least 98% most preferably at least 99% over a sequence of at least 250, 300, 400, 500, 600 preferably 700, more preferably 800, even more preferably 900, even more preferably 1000, even more preferably 1100, even more preferably 1200, even more preferably 1300, even more preferably 1400 consecutive nucleic acid base pairs, most preferably the entire length of SEQ ID NO: 16, 17, 20 or 21, and;
(iii) a sequence that hybridizes under medium stringency conditions, preferably high stringency conditions, to a sequence of (i) or (ii) or a complementary sequence thereto; and
(iv) a fragment of at least 100 consecutive bases, preferably at least 200, 300, 400 or 500 consecutive bases, more preferably at least 600, 700, or 800 consecutive bases, even more preferably at least 900, 1000, 1100, 1200, 1300 or 1400 consecutive bases of a nucleic acid molecule of i) to iii), in a most preferred embodiment the fragments are comprising the 3′ end of the sequence of SEQ ID NO: 16, 17, 20 or 21, (i) to (iii)
   wherein the promoters comprising a sequence as defined in (ii) to (iv) are conferring expression in a microorganism of the phylum Ascomycota.

The promoter comprising SEQ ID NO: 16, 17, 20 or 21 and functional fragments and derivative thereof surprisingly lead to robust and strong expression of heterologous nucleic acid molecules operably linked thereto in the respective fungal cell. The promoters of the invention significantly outperformed the promoter Pcbh1 (SEQ ID NO: 22 and 23) and Pegl5 (SEQ ID NO: 18 and 19) used as standard promotor for expression of heterologous nucleic acid molecules in the respective fungal cell (Visser et al 2011, Industrial Biotechnology 7 (3), US2014127788). Hence, in a further embodiment of the invention, the promoter comprising SEQ ID NO: 16, 17, 20 or 21 and functional fragments and derivatives thereof direct expression of a heterologous nucleic acid molecule operably linked thereto statistically significantly higher than the Pcbh1 promoter operably linked to the same heterologous sequence in the same genetic locus of a fungal cell grown under comparable conditions. Preferably, the expression derived from the promoters of the invention is at least 1,1 time, preferably 1,2 time, more preferably 1,3 time, more preferably at least 1,4 time higher than the expression derived from the Pcbh1 promoter (SEQ ID NO: 22 or 23).

A further embodiment of the invention is an expression construct comprising an isolated promoter as defined above, operably linked to a heterologous nucleic acid. The heterologous nucleic acid may comprise an ORF, a functional RNA and/or a regulatory sequence e.g. terminator or enhancer.

A further embodiment of the invention is an expression vector comprising an isolated promoter or the expression construct as defined above. The vector may be a cloning vector or an expression vector and may be selected from plasmids suitable for transformation of filamentous fungal cells, preferably Thermothelomyces thermophilus cells, and in particular plasmids suitable for expression of proteins in filamentous fungal cells, preferably Thermothelomyces thermophilus cells, e.g. plasmids which are capable of autonomous replication in other organisms, preferably in bacteria, in particular E. coli, and which can be prepared, e.g. digested, for genomic insertional transformation of filamentous fungal cells, preferably Thermothelomyces thermophilus cells.

A method for producing an expression construct, said method comprising operably linking an isolated promoter as defined above to a heterologous nucleic acid is also enclosed in the invention at hand. The heterologous nucleic acid may be a sequence to be expressed in a fungal host cell, such as a nucleic acid molecule encoding a polypeptide or protein or a functional RNA. The heterologous nucleic acid may further comprise an additional regulatory sequence such as an enhancer or terminator.

A further embodiment of the invention is a process for producing a vector, said method comprising linking an isolated promoter or an expression construct as defined above to a vector. According to the invention, the isolated promoter may be used for the production of an expression construct and both the isolated promoter as defined above and the expression construct as defined above may be used for the production of an expression vector.

Microorganisms of the phylum Ascomycota, preferably Acremonium, Aspergillus, Agaricus, Aureobasidium, Cryptococcus, Corynascus, Chrysosporium, Filibasidium, Fusarium, Humicola, Magnaporthe, Monascus, Mucor, Myceliophthora, Mortierella, Neocallimastix, Neurospora, Paecilomyces, Penicillium, Piromyces, Phanerochaete, Podospora, Pycnoporus, Rhizopus, Schizophyllum, Sordaria, Talaromyces, Rasamsonia, Thermoascus, Thermothelomyces, Thielavia, Tolypocladium, Trametes and Trichoderma, more preferred Aspergillus niger, Aspergillus oryzae, Aspergillus fumigatus, Neurospora crassa, Penicillium chrysogenum, Penicillium citrinum, Acremonium chrysogenum, Trichoderma reesei, Rasamsonia emersonii (formerly known as Talaromyces emersonii), Aspergillus sojae, Thermothelomyces heterothallica and Thermothelomyces thermophilus (formerly known as Myceliophthora thermophila and as Chrysosporium lucknowense), most preferably Thermothelomyces thermophilus, comprising an expression construct or an expression vector as defined above are encompassed by the invention at hand. Preferably the expression construct or expression vector of the invention is integrated into the genome of said microorganism.

A further embodiment of the invention is a method for producing a transgenic microorganism of the phylum Ascomycota, preferably Acremonium, Aspergillus, Agaricus, Aureobasidium, Cryptococcus, Corynascus, Chrysosporium, Filibasidium, Fusarium, Humicola, Magnaporthe, Monascus, Mucor, Myceliophthora, Mortierella, Neocallimastix, Neurospora, Paecilomyces, Penicillium, Piromyces, Phanerochaete, Podospora, Pycnoporus, Rhizopus, Schizophyllum, Sordaria, Talaromyces, Rasamsonia, Thermoascus, Thermothelomyces, Thielavia, Tolypocladium, Trametes and Trichoderma, more preferred Aspergillus niger, Aspergillus oryzae, Aspergillus fumigatus, Neurospora crassa, Penicillium chrysogenum, Penicillium citrinum, Acremonium chrysogenum, Trichoderma reesei, Rasamsonia emersonii (formerly known as Talaromyces emersonii), Aspergillus sojae, Thermothelomyces heterothallica and Thermothelomyces thermophilus (formerly known as Myceliophthora thermophila and as Chrysosporium lucknowense), most preferably Thermothelomyces thermophilus, said method comprising introducing an isolated promoter or an expression construct or expression vector of the invention into said microorganism, preferably into the genome of said microorganism.

In a further embodiment of the invention the isolated promoter of the invention is operably linked to a heterologous nucleic acid encoding a protein selected from the group consisting of hydrolase, isomerase, ligase, lyase, oxidoreductase or transferase, preferably aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, phytase, polyphneoloxidase, proteolytic enzyme, ribonuclease, transglutaminase, xylanase and beta-xylosidase.

A further embodiment of the invention is a method for expression of a heterologous nucleic acid in a recombinant microorganism of the phylum Ascomycota, preferably Acremonium, Aspergillus, Agaricus, Aureobasidium, Cryptococcus, Corynascus, Chrysosporium, Filibasidium, Fusarium, Humicola, Magnaporthe, Monascus, Mucor, Myceliophthora, Mortierella, Neocallimastix, Neurospora, Paecilomyces, Penicillium, Piromyces, Phanerochaete, Podospora, Pycnoporus, Rhizopus, Schizophyllum, Sordaria, Talaromyces, Rasamsonia, Thermoascus, Thermothelomyces, Thielavia, Tolypocladium, Trametes and Trichoderma, more preferred Aspergillus niger, Aspergillus oryzae, Aspergillus fumigatus, Neurospora crassa, Penicillium chrysogenum, Penicillium citrinum, Acremonium chrysogenum, Trichoderma reesei, Rasamsonia emersonii (formerly known as Talaromyces emersonii), Aspergillus sojae, Thermothelomyces heterothallica and Thermothelomyces thermophilus (formerly known as Myceliophthora thermophila and as Chrysosporium lucknowense), most preferably Thermothelomyces thermophilus, comprising the step of cultivating a recombinant microorganism of the invention in a medium conducive for the expression of said heterologous nucleic acid, wherein the microorganism comprises a promoter according as defined above operably linked to said heterologous nucleic acid, preferably wherein the heterologous nucleic acid encodes any of a hydrolase, isomerase, ligase, lyase, oxidoreductase or transferase, preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, phytase, polyphneoloxidase, proteolytic enzyme, ribonuclease, transglutaminase, xylanase or beta-xylosidase.

A further embodiment of the invention is a method for the production of a target protein in a recombinant microorganism of the phylum Ascomycota, comprising the steps of
(i) cultivating said recombinant microorganism of the phylum Ascomycota in a medium conducive for the expression of the protein, wherein said recombinant microorganism comprises a promoter according to the invention operably linked to a heterologous nucleic acid encoding said target protein, and
(ii) recovering said target protein, wherein the recombinant microorganism of the phylum Ascomycota preferably is Acremonium, Aspergillus, Agaricus, Aureobasidium, Cryptococcus, Corynascus, Chrysosporium, Filibasidium, Fusarium, Humicola, Magnaporthe, Monascus, Mucor, Myceliophthora, Mortierella, Neocallimastix, Neurospora, Paecilomyces, Penicillium, Piromyces, Phanerochaete, Podospora, Pycnoporus, Rhizopus, Schizophyllum, Sordaria, Talaromyces, Rasamsonia, Thermoascus, Thermothelomyces, Thielavia, Tolypocladium, Trametes and Trichoderma, more preferred Aspergillus niger, Aspergillus oryzae, Aspergillus fumigatus, Neurospora crassa, Penicillium chrysogenum, Penicillium citrinum, Acremonium chrysogenum, Trichoderma reesei, Rasamsonia emersonii (formerly known as Talaromyces emersonii), Aspergillus sojae, Thermothelomyces heterothallica and Thermothelomyces thermophilus (formerly known as Myceliophthora thermophila and as Chrysosporium lucknowense), most preferably Thermothelomyces thermophilus, and said target protein is a hydrolase, isomerase, ligase, lyase, oxidoreductase or transferase, preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, phytase, polyphneoloxidase, proteolytic enzyme, ribonuclease, transglutaminase, xylanase or beta-xylosidase.

The recovery process of any fermentative or biocatalytically produced peptide, protein or fine chemical comprises any suitable method for separating the recombinant polypeptide from the so-called “biomass” and ingredients of the culture medium. Suitable separation techniques known in the art include, but are not limited to, filtration, microfiltration, ultrafiltration, centrifugation, extraction, spray drying, evaporation, freeze drying and precipitation. The recombinant polypeptide may further be purified by a variety of procedures known in the art including, but not limited to, ammonium sulfate precipitation or other protein precipitation methods, ion exchange chromatography, affinity chromatography, hydrophobic interaction chromatography, size exclusion chromatography or electrophoretic procedures.

### Definitions

It is to be understood that this invention is not limited to the particular methodology or protocols. It is also to be understood that the terminology used herein is for the purpose of describing particular embodiments only and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. For clarity, certain terms used in the specification are defined and used as follows: Coding region: As used herein the terms “coding region” or “open reading frame” or “ORF” when used in reference to a structural gene refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine and on the 3′-side by one of the three triplets which specify stop codons (i.e., TAA, TAG, TGA). In addition to containing introns, genomic forms of a gene may also include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, post-transcriptional cleavage and polyadenylation.

Complementary: “Complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT-3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules. The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

Endogenous: An “endogenous” nucleotide sequence refers to a nucleotide sequence, which is present in the genome of the untransformed cell.

Enhanced expression: “enhance” or “increase” the expression of a nucleic acid molecule in a cell are used equivalently herein and mean that the level of expression of the nucleic acid molecule in a cell after applying a method of the present invention is higher than its expression in the cell before applying the method, or compared to a reference cell lacking a recombinant nucleic acid molecule of the invention. The term “enhanced” or “increased” as used herein are synonymous and means herein higher, preferably significantly higher expression of the nucleic acid molecule to be expressed. As used herein, an “enhancement” or “increase” of the level of an agent such as a protein, mRNA or RNA means that the level is increased relative to a substantially identical cell grown under substantially identical conditions, lacking a recombinant nucleic acid molecule of the invention, the recombinant construct or recombinant vector of the invention. As used herein, “enhancement” or “increase” of the level of an agent, such as for example a preRNA, mRNA, rRNA, tRNA, snoRNA, snRNA expressed by the target gene and/or of the protein product encoded by it, means that the level is increased 50% or more, for example 100% or more, preferably 200% or more, more preferably 5 fold or more, even more preferably 10 fold or more, most preferably 20 fold or more for example 50 fold relative to a cell or organism lacking a recombinant nucleic acid molecule of the invention. The enhancement or increase can be determined by methods with which the skilled worker is familiar. Thus, the enhancement or increase of the nucleic acid or protein quantity can be determined for example by an immuno-logical detection of the protein. Moreover, techniques such as protein assay, fluorescence, Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be employed to measure a specific protein or RNA in a cell. Depending on the type of the induced protein product, its activity or the effect on the phenotype of the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford M M (1976) Analyt Biochem 72:248-254).

Expression: “Expression” refers to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. In other cases, expression may refer only to the transcription of the DNA harboring an RNA molecule.

Expression construct: “Expression construct” as used herein mean a DNA sequence capable of directing expression of a particular nucleotide sequence in a cell, comprising a promoter functional in said cell into which it will be introduced, operatively linked to the nucleotide sequence of interest which is—optionally—operatively linked to termination signals. If translation is required, it also typically comprises sequences required for proper translation of the nucleotide sequence. The coding region may code for a protein of interest but may also code for a functional RNA of interest, for example RNAa, siRNA, snoRNA, snRNA, microRNA, ta-siRNA or any other noncoding regulatory RNA, in the sense or antisense direction. The expression construct comprising the nucleotide sequence of interest may be chimeric, meaning that one or more of its components is heterologous with respect to one or more of its other components. The expression construct may also be one, which is naturally occurring but has been obtained in a recombinant form useful for heterologous expression. Typically, however, the expression construct is heterologous with respect to the host, i.e., the particular DNA sequence of the expression construct does not occur naturally in the host cell and must have been introduced into the host cell or an ancestor of the host cell by a transformation event. The expression of the nucleotide sequence in the expression construct may be under the control of a constitutive promoter or of an inducible promoter, which initiates transcription only when the host cell is exposed to some particular external stimulus. The promoter can also be specific to a particular stage of development.

Foreign: The term “foreign” refers to any nucleic acid molecule (e.g., gene sequence) which is introduced into the genome of a cell by experimental manipulations and may include sequences found in that cell so long as the introduced sequence contains some modification (e.g., a point mutation, the presence of a selectable marker gene, etc.) and is therefore distinct relative to the naturally-occurring sequence.

Functional linkage: The term “functional linkage” or “functionally linked” are to be understood as meaning, for example, the sequential arrangement of a regulatory element (e.g. a promoter) with a nucleic acid sequence to be expressed and, if appropriate, further regulatory elements (such as e.g., a terminator) in such a way that each of the regulatory elements can fulfil its intended function to allow, modify, facilitate or otherwise influence expression of said nucleic acid sequence. As a synonym the wording “operable linkage” or “operably linked” may be used. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. In a preferred embodiment, the nucleic acid sequence to be transcribed is located behind the promoter in such a way that the transcription start is identical with the desired beginning of the chimeric RNA of the invention. Functional linkage, and an expression construct, can be generated by means of customary recombination and cloning techniques as described (e.g., in Maniatis T, Fritsch E F and Sambrook J (1989) Molecular Cloning: A Laboratory Manual, 2nd Ed., Cold Spring Harbor Laboratory, Cold Spring Harbor (NY); Silhavy et al. (1984) Experiments with Gene Fusions, Cold Spring Harbor Laboratory, Cold Spring Harbor (NY); Ausubel et al. (1987) Current Protocols in Molecular Biology, Greene Publishing Assoc, and Wiley Interscience). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form and be inserted into the genome, for example by transformation.

Gene: The term “gene” refers to a region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

Genome and genomic DNA: The terms “genome” or “genomic DNA” is referring to the heritable genetic information of a host organism. Preferably the terms genome or genomic DNA is referring to the chromosomal DNA of the nucleus.

Heterologous: The term “heterologous” with respect to a nucleic acid molecule or DNA refers to a nucleic acid molecule which is operably linked to, or is manipulated to become operably linked to, a second nucleic acid molecule, e.g. a promoter to which it is not operably linked in nature, e.g. in the genome of a WT cell, or to which it is operably linked at a different location or position in nature, e.g. in the genome of a WT cell.

Preferably the term “heterologous” with respect to a nucleic acid molecule or DNA, e.g. promoter refers to a nucleic acid molecule which is operably linked to, or is manipulated to become operably linked to, a second nucleic acid molecule, e.g. a ORF to which it is not operably linked in nature.

A heterologous expression construct comprising a nucleic acid molecule and one or more regulatory nucleic acid molecule (such as a promoter or a transcription termination signal) linked thereto for example is a constructs originating by experimental manipulations in which either a) said nucleic acid molecule, or b) said regulatory nucleic acid molecule or c) both (i.e. (a) and (b)) is not located in its natural (native) genetic environment or has been modified by experimental manipulations, an example of a modification being a substitution, addition, deletion, in-version or insertion of one or more nucleotide residues. Natural genetic environment refers to the natural chromosomal locus in the organism of origin, or to the presence in a genomic library. In the case of a genomic library, the natural genetic environment of the sequence of the nucleic acid molecule is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815). For example, a protein encoding nucleic acid molecule operably linked to a promoter, which is not the native promoter of this molecule, is considered to be heterologous with respect to the promoter. Preferably, heterologous DNA is not endogenous to or not naturally associated with the cell into which it is introduced, but has been obtained from another cell or has been synthesized. Heterologous DNA also includes an endogenous DNA sequence, which contains some modification, non-naturally occurring, multiple copies of an endogenous DNA sequence, or a DNA sequence which is not naturally associated with another DNA sequence physically linked thereto.

Hybridization: The term “hybridization” as defined herein is a process wherein substantially complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids. The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tm) for the specific sequence at a defined ionic strength and pH. Medium stringency conditions are when the temperature is 20° C. below Tm, and high stringency conditions are when the temperature is 10° C. below Tm. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore, medium stringency hybridisation conditions may sometimes be needed to identify such nucleic acid molecules.

The “Tm” is the temperature under defined ionic strength and pH, at which 50% of the target sequence hybridises to a perfectly matched probe. The Tm is dependent upon the solution conditions and the base composition and length of the probe. For example, longer sequences hybridise specifically at higher temperatures. The maximum rate of hybridisation is obtained from about 16° C. up to 32° C. below Tm. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:

DNA-DNA hybrids (Meinkoth and Wahl, Anal. Biochem., 138:267-284, 1984):

$$Tm = 81.5 ° C . + 16.6 \times log [ Na + ] a + 0.41 \times \% [ G / Cb ] - 500 \times [ Lc ] - 1 - 0.61 \times \% formamide$$

DNA-RNA or RNA-RNA hybrids:

$$Tm = 79.8 + 18.5 ( log 10 [ Na + ] a ) + 0.58 ( \% G / Cb ) + 11.8 ( \% G / Cb ) 2 - 820 / Lc$$

oligo-DNA or oligo-RNAd hybrids:

$$\begin{matrix}For < 20 nucleotides : & Tm = 2 ( ln ) \\ For 20 - 35 nucleotides : & Tm = 22 + 1.46 ( ln )\end{matrix}$$

a or for other monovalent cation, but only accurate in the 0.01-0.4 M range.

b only accurate for % GC in the 30% to 75% range.

$$c L = length of duplex in base pairs .$$

d Oligo, oligonucleotide; In, effective length of primer=2×(no. of G/C)+ (no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-related probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate. Another example of high stringency conditions is hybridisation at 65° C. in 0.1×SSC comprising 0.1 SDS and optionally 5×Denhardt's reagent, 100 μg/ml denatured, fragmented salmon sperm DNA, 0.5% sodium pyrophosphate, followed by the washing at 65° C. in 0.3×SSC.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

“Identity”: “Identity” when used in respect to the comparison of two or more nucleic acid or amino acid molecules means that the sequences of said molecules share a certain degree of sequence similarity, the sequences being partially identical.

Enzyme variants may be defined by their sequence identity when compared to a parent enzyme or nucleic acid molecule. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapo-pen=10.0, gapextend=0.5 and matrix=EBLOSUM62). The preferred alignment for the purpose of this invention is that alignment, from which the highest sequence identity can be determined. The following example is meant to illustrate two nucleotide sequences, but the same calculations apply to protein sequences:

Seq A:
AAGATACTG
length: 9 bases
Seq B:
GATCTGA
length: 7 bases

Hence, the shorter sequence is sequence B.

Producing a pairwise global alignment which is showing both sequences over their complete lengths results in

| | |
|---|---|
| Seq A: | AAGATACTG- |
| | \|\|\| \|\|\| |
| Seq B: | --GAT-CTGA |

The “I” symbol in the alignment indicates identical residues (which means bases for DNA or amino acids for proteins). The number of identical residues is 6.

The “-” symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

| | |
|---|---|
| Seq A: | GATACTG- |
| | \|\|\| \|\|\| |
| Seq B: | GAT-CTGA |

Producing a pairwise alignment which is showing sequence A over its complete length according to the invention consequently results in:

| | |
|---|---|
| Seq A: | AAGATACTG |
| | \|\|\| \|\|\| |
| Seq B: | --GAT-CTG |

Producing a pairwise alignment which is showing sequence B over its complete length according to the invention consequently results in:

| | |
|---|---|
| Seq A: | GATACTG- |
| | \|\|\| \|\|\| |
| Seq B: | GAT-CTGA |

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).

Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %-identity=(identical residues/length of the alignment region which is showing the respective sequence of this invention over its complete length)*100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”. According to the example provided above, %-identity is: for Seq A being the sequence of the invention (6/9)*100=66.7%; for Seq B being the sequence of the invention (6/8)*100=75%.

The term “Introducing”, “introduction” and the like with respect to the introduction of a donor DNA molecule in the target site of a target DNA means any introduction of the sequence of the donor DNA molecule into the target region for example by the physical integration of the donor DNA molecule or a part thereof into the target region or the introduction of the sequence of the donor DNA molecule or a part thereof into the target region wherein the donor DNA is used as template for a polymerase.

Isogenic: organisms (e.g., fungi), which are genetically identical, except that they may differ by the presence or absence of a heterologous DNA sequence.

Isolated: The term “isolated” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring polynucleotide or polypeptide present in a living cell is not isolated, but the same polynucleotide or polypeptide, separated from some or all of the coexisting materials in the natural system, is isolated. Such polynucleotides can be part of a vector and/or such polynucleotides or polypeptides could be part of a composition and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighbouring genes; RNA sequences, such as a specific mRNA sequence encoding a specific protein, are found in the cell as a mixture with numerous other mRNAs, which encode a multitude of proteins. However, an isolated nucleic acid sequence comprising for example SEQ ID NO: 1 includes, by way of example, such nucleic acid sequences in cells which ordinarily contain SEQ ID NO: 1 where the nucleic acid sequence is in a chromosomal or extrachromosomal location different from that of natural cells or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

Minimal Promoter: promoter elements, particularly a TATA element, that are inactive or that have greatly reduced promoter activity in the absence of upstream activation. In the presence of a suitable transcription factor, the minimal promoter functions to permit transcription.

Non-coding: The term “non-coding” refers to sequences of nucleic acid molecules that do not encode part or all of an expressed protein. Non-coding sequences include but are not limited to functional RNAs, introns, enhancers, promoter regions, 3′ untranslated regions, and 5′ untranslated regions.

Nucleic acids and nucleotides: The terms “Nucleic Acids” and “Nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used interchangeably herein with “gene”, “cDNA, “mRNA”, “oligonucleotide,” and “polynucleotide”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

Nucleic acid sequence: The phrase “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein.

Oligonucleotide: The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Overhang: An “overhang” is a relatively short single-stranded nucleotide sequence on the 5′- or 3′-hydroxyl end of a double-stranded oligonucleotide molecule (also referred to as an “extension,” “protruding end,” or “sticky end”).

Polypeptide: The terms “polypeptide”, “peptide”, “oligopeptide”, “polypeptide”, “gene product”, “expression product” and “protein” are used interchangeably herein to refer to a polymer or oligomer of consecutive amino acid residues.

“Precise” with respect to the introduction of a donor DNA molecule in target region means that the sequence of the donor DNA molecule is introduced into the target region without any InDels, duplications or other mutations as compared to the unaltered DNA sequence of the target region that are not comprised in the donor DNA molecule sequence.

Promoter: The terms “promoter”, or “promoter sequence” are equivalents and as used herein, refer to a DNA sequence which when operably linked to a nucleotide sequence of interest is capable of controlling the transcription of the nucleotide sequence of interest into RNA. A promoter is located 5′ (i.e., upstream), proximal to the transcriptional start site of a nucleotide sequence of interest whose transcription into RNA it controls, and provides a site for specific binding by RNA polymerase and other transcription factors for initiation of transcription. Said promoter comprises for example the at least 10 kb, for example 5 kb or 2 kb proximal to the transcription start site. It may also comprise the at least 1500 bp proximal to the transcriptional start site, preferably the at least 1000 bp. In a further preferred embodiment, the promoter comprises the at least 50 bp proximal to the transcription start site, for example, at least 25 bp. The promoter does not comprise exon and/or intron regions or 5′ untranslated regions. The promoter may for example be heterologous or homologous to the respective cell. A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). Suitable promoters can be derived from genes of the host cells where expression should occur or from pathogens for this host cells. Activity of a promoter may be evaluated by, for example, operably linking a reporter gene to the promoter sequence to generate a reporter construct, introducing the reporter construct into the genome of a cell and detecting the expression of the reporter gene (e.g., detecting mRNA, protein, or the activity of a protein encoded by the reporter gene).

Purified: As used herein, the term “purified” refers to molecules, either nucleic or amino acid sequences that are removed from their natural environment, isolated or separated. “Substantially purified” molecules are at least 60% free, preferably at least 75% free, and more preferably at least 90% free from other components with which they are naturally associated. A purified nucleic acid sequence may be an isolated nucleic acid sequence.

Recombinant: The term “recombinant” with respect to nucleic acid molecules refers to nucleic acid molecules produced by recombinant DNA techniques. Recombinant nucleic acid molecules may also comprise molecules, which as such does not exist in nature but are modified, changed, mutated or otherwise manipulated by man. Preferably, a “recombinant nucleic acid molecule” is a non-naturally occurring nucleic acid molecule that differs in sequence from a naturally occurring nucleic acid molecule by at least one nucleic acid. A “recombinant nucleic acid molecule” may also comprise a “recombinant construct” which comprises, preferably operably linked, a sequence of nucleic acid molecules not naturally occurring in that order. Preferred methods for producing said recombinant nucleic acid molecule may comprise cloning techniques, directed or non-directed mutagenesis, synthesis or recombination techniques.

Sense: The term “sense” is understood to mean a nucleic acid molecule having a sequence which is complementary or identical to a target sequence, for example a sequence which binds to a protein transcription factor and which is involved in the expression of a given gene. According to a preferred embodiment, the nucleic acid molecule comprises a gene of interest and elements allowing the expression of the said gene of interest.

Significant increase or decrease: An increase or decrease, for example in enzymatic activity or in gene expression, that is larger than the margin of error inherent in the measurement technique, preferably an increase or decrease by about 2-fold or greater of the activity of the control enzyme or expression in the control cell, more preferably an increase or decrease by about 5-fold or greater, and most preferably an increase or decrease by about 10-fold or greater.

Small nucleic acid molecules: “small nucleic acid molecules” are understood as molecules consisting of nucleic acids or derivatives thereof such as RNA or DNA. They may be double-stranded or single-stranded and are between about 15 and about 30 bp, for example between 15 and 30 bp, more preferred between about 19 and about 26 bp, for example between 19 and 26 bp, even more preferred between about 20 and about 25 bp for example between 20 and 25 bp. In an especially preferred embodiment, the oligonucleotides are between about 21 and about 24 bp, for example between 21 and 24 bp. In a most preferred embodiment, the small nucleic acid molecules are about 21 bp and about 24 bp, for example 21 bp and 24 bp.

Substantially complementary: In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the latter being equivalent to the term “identical” in this context). Preferably identity is assessed over a length of at least 19 nucleotides, preferably at least 50 nucleotides, more preferably the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48:443-453; as defined above). A nucleotide sequence “substantially complementary” to a reference nucleotide sequence hybridizes to the reference nucleotide sequence under low stringency conditions, preferably medium stringency conditions, most preferably high stringency conditions (as defined above).

“Target site” as used herein means the position in the genome at which a double strand break or one or a pair of single strand breaks (nicks) are induced using recombinant technologies such as Zn-finger, TALEN, restriction enzymes, homing endonucleases, RNA-guided nucleases, RNA-guided nickases such as CRISPR/Cas nucleases or nickases and the like.

Transgene: The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

Transgenic: The term transgenic when referring to an organism means transformed, preferably stably transformed, with a recombinant DNA molecule that preferably comprises a suitable promoter operatively linked to a DNA sequence of interest.

Vector: As used herein, the term “vector” refers to a nucleic acid molecule capable of transporting another nucleic acid molecule to which it has been linked. One type of vector is a genomic integrated vector, or “integrated vector”, which can become integrated into the chromosomal DNA of the host cell. Another type of vector is an episomal vector, i.e., a nucleic acid molecule capable of extra-chromosomal replication. Vectors capable of directing the expression of genes to which they are operatively linked are referred to herein as “expression vectors”. In the present specification, “plasmid” and “vector” are used interchangeably unless otherwise clear from the context. Expression vectors designed to produce RNAs as described herein in vitro or in vivo may contain sequences recognized by any RNA polymerase, including mitochondrial RNA polymerase, RNA pol I, RNA pol II, and RNA pol III. These vectors can be used to transcribe the desired RNA molecule in the cell according to this invention.

Wild-type: The term “wild-type”, “natural” or “natural origin” means with respect to an organism, polypeptide, or nucleic acid sequence, that said organism is naturally occurring or available in at least one naturally occurring organism which is not changed, mutated, or otherwise manipulated by man.

### FIGURES

FIG. 1 shows the total phytase activity of the different phytase production strains in relation to the activity measured for UV18 #100f Δpyr5 Δalp1 Δku70 cbh1::Pegl5 (1.3 kb)-phytase-Tcbh1 which was set to 100%. UV18 #100f Δpyr5 Δalp1 Δku70 cbh1::Pcbh3 (1.6 kb)-phytase-Tcbh1 (overexpression of phytase gene driven by 1.6 kb cbh3 promoter)/UV18 #100f Δpyr5 Δalp1 Δku70 cbh1::Pcbh3 (2.1 kb)-phytase-Tcbh1 (overexpression of phytase gene driven by 2.1 kb cbh3 promoter)/UV18 #100f Δpyr5 Δalp1 Δku70 cbh1::Pegl5 (1.2 kb)-phytase-Tcbh1 (overexpression of phytase gene driven by 1.2 kb eg/5 promoter)/UV18 #100f Δpyr5 Δalp1 Δku70 cbh1::Peg15 (1.3 kb)-phytase-Tcbh1 (overexpression of phytase gene driven by 1.3 kb egl5 promoter)/UV18 #100f Δpyr5 Δalp1 Δku70 cbh1::Pcbh2 (1.6 kb)-phytase-Tcbh1 (overexpression of phytase gene driven by 1.6 kb cbh2 promoter)/UV18 #100f Δpyr5 Δalp1 Δku70 cbh1::Pcbh2 (2.4 kb)-phytase-Tcbh1 (overexpression of phytase gene driven by 2.4 kb cbh2 promoter)/UV18 #100f Δpyr5 Δalp1 Δku70 cbh1::Pcbh1 (1.5 kb)-phytase-Tcbh1 (overexpression of phytase gene driven by 1.5 kb cbh1 promoter)/UV18 #100f Δpyr5 Δalp1 Δku70 cbh1::Pcbh1 (2.0 kb)-phytase-Tcbh1 (overexpression of phytase gene driven by 2.0 kb cbh1 promoter).

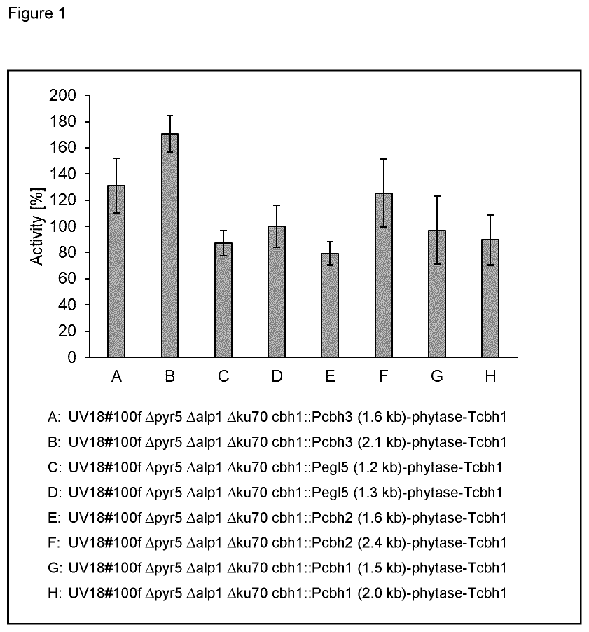

FIG. 2 shows an SDS-PAGE analysis of equal amounts of protein from the supernatant of different phytase production strains in comparison to the parental strain. UV18 #100f Δpyr5 Δalp1 Δku70 cbh1::Pcbh3 (1.6 kb)-phytase-Tcbh1 (overexpression of phytase gene driven by 1.6 kb cbh3 promoter)/UV18 #100f Δpyr5 Δalp1 Δku70 cbh1::Pcbh3 (2.1 kb)-phytase-Tcbh1 (overexpression of phytase gene driven by 2.1 kb cbh3 promoter)/UV18 #100f Δpyr5 Δalp1 Δku70 cbh1::Pcbh2 (1.6 kb)-phytase-Tcbh1 (overexpression of phytase gene driven by 1.6 kb cbh2 promoter)/UV18 #100f Δpyr5 Δalp1 Δku70 cbh1::Pcbh2 (2.4 kb)-phytase-Tcbh1 (overexpression of phytase gene driven by 2.4 kb cbh2 promoter)/UV18 #100f Δpyr5 Δalp1 Δku70 cbh1::Pcbh1 (1.5 kb)-phytase-Tcbh1 (overexpression of phytase gene driven by 1.5 kb cbh1 promoter)/UV18 #100f Δpyr5 Δalp1 Δku70 cbh1::Pcbh1 (2.0 kb)-phytase-Tcbh1 (overexpression of phytase gene driven by 2.0 kb cbh1 promoter)/UV18 #100f Δpyr5 Δalp1 Δku70 cbh1::Pegl5 (1.2 kb)-phytase-Tcbh1 (overexpression of phytase gene driven by 1.2 kb eg/5 promoter)/UV18 #100f Δpyr5 Δalp1 Δku70 cbh1::Peg15 (1.3 kb)-phytase-Tcbh1 (overexpression of phytase gene driven by 1.3 kb eg/5 promoter)/UV18 #100f Δpyr5 Δalp1 Δku70 (parental strain). The glycosylated phytase runs at approx. 55 kDa in the SDS gel (indicated with an arrow).

### EXAMPLES

### Chemicals and Common Methods

Unless indicated otherwise, cloning procedures carried out for the purposes of the present invention including restriction digest, agarose gel electrophoresis, purification of nucleic acids, Ligation of nucleic acids, transformation, selection and cultivation of bacterial cells were performed as described (Sambrook et al., 1989). Sequence analyses of recombinant DNA were performed with a laser fluorescence DNA sequencer (Applied Biosystems, Foster City, CA, USA) using the Sanger technology (Sanger et al., 1977). Unless described otherwise, chemicals and reagents were obtained from Sigma Aldrich (Sigma Aldrich, St. Louis, USA), from Promega (Madison, WI, USA), Duchefa (Haarlem, The Netherlands) or Invitrogen (Carlsbad, CA, USA). Restriction endonucleases were from New England Biolabs (Ipswich, MA, USA). Oligonucleotides were synthesized by Integrated DNA Technologies (Coralville, IA, USA).

### Example 1

Transformation of Thermothelomyces thermophilus

Several methods for the transformation of T. thermophilus protoplasts are described in the literature (WO 00/20555, US 2012/0005812, Verdoes et al. (2007) Industrial Biotechnology 3(1): 48-57).

Protoplasts of T. thermophilus strains were prepared by inoculating a 25 ml preculture of a standard fungal growth media with 0.7-1×10⁵ spores/ml in a 100 ml shake flask for 24 h at 37° C. and 250 rpm. The main culture was prepared by inoculating 100 ml of a standard fungal growth media with 20 ml of the preculture in a 500 ml shake flask for 24 h at 37° C. and 250 rpm. The mycelium was harvested by filtration through a sterile Cell Strainer (VWR) and washed with 100 ml 2000 mosmol/l NaCl/CaCl₂) (0.6 M NaCl and 0.27 M CaCl₂*H₂O). 1 g of the washed mycelium was transferred into a 100 ml flask. The mycelium was mixed with 40 mg Glucanex solution (2.5 mg/ml in 2000 mosmol/l NaCl/CaCl₂)) and 10 mg Yatalase solution (0.625 mg/ml in 2000 mos-mol/l NaCl/CaCl₂)) and 16 ml of 2000 mosmol/l NaCl/CaCl₂). The mycelium suspension was incubated at 30° C. and 70 rpm for 50-70 min until protoplasts are visible under the microscope.

Harvesting of protoplasts was done by filtration through a sterile Cell Strainer into a sterile 50 ml tube. After the addition of 25 ml ice-cold STC solution (1.2 M sorbitol, 50 mM CaCl₂), 35 mM NaCl, 10 mM Tris/HCl pH 7.5) to the flow through, the protoplasts were harvested by centrifugation (1200×g, 10 min, 4° C.). The protoplasts were washed again in 50 ml STC and resuspended in 0.5-1.2 ml STC.

For transformation, 5-10 μg of linearized DNA, 1 μl 0.5 M aurintricarboxylic acid (ATA) and 100 μl of protoplast suspension were mixed and incubated for 30-40 min on ice. Then 1.7 ml of PEG solution (60% PEG4000 [polyethylenglycol], 50 mM CaCl₂, 35 mM NaCl, 10 mM Tris/HCl pH 7.5) was added and mixed gently. After incubation for 30 min at 4° C., the tube was filled with 11 ml STC solution, centrifuged (900×g, 10 min, 4° C.), and the supernatant discarded. The pellet was resuspended in the remaining STC and plated on selective media plates as known in the art. After incubation of the plates for 3-6 days at 37° C., transformants were picked and re-streaked on selective media.

### Selective Media Plates

If the pyr5 gene is used as selection marker, enriched minimal medium without uridine and uracil is used to select positive transformants (sucrose is only added in case protoplasts are plated):

| | | | |
|---|---|---|---|
| Glucose | 10 | g/l | |
| Sucrose | 230 | g/l | |
| Mg₂SO₄*7H₂O | 0.49 | g/l | |
| KCl | 0.52 | g/l | |
| KH₂PO₄ | 1.52 | g/l | |
| NaNO₃ | 6 | g/l | |
| CuSO₄*5H₂O | 1.6 | mg/l | |
| FeSO₄*7H₂O | 5 | mg/l | |
| ZnSO₄*7H₂O | 22 | mg/l | |
| MnSO₄*H₂O | 4.3 | mg/l | |
| CoCl₂*6H₂O | 1.6 | mg/l | |
| Na₂MoO₄*2H₂O | 1.5 | mg/l | |
| H₃BO₃ | 11 | mg/l | |
| EDTA | 50 | mg/l | |
| Penicillin | 50000 | U/l | |
| Streptomycin | 50 | mg/l | |
| Casaminoacids | 1 | g/l | |
| Agar | 16 | g/l | set pH to 6.5 |

### Example 2

Generation of Phytase Expression Plasmids with Different Cellulase Promoters Expression Construct with Cbh2 Promoter

A synthetic gene (GeneArt, ThermoFisher Scientific Inc., USA) (SEQ ID NO: 1) encoding a synthetic phytase from bacterial origin (disclosed in WO 2012/143862; as phytase PhV-99; SEQ ID NO. 2) was used for the construction of a phytase expression plasmids. For the secretion of the phytase, a signal sequence encoding for a signal peptide derived from T. thermophilus was added to the mature sequence of the phytase. A promotor sequence amplified from the up-stream region of the Cbh2 encoding gene (MYCTH: XP_003661032.1) and a terminator sequence amplified from the downstream region of the Cbh1 encoding gene from T. thermophilus were used as regulatory elements to drive the expression of the phytase. Using standard cloning techniques, the expression plasmid pMT2194 (SEQ ID NO: 4) was constructed based on the E. coli standard cloning vector MT940 (SEQ ID NO: 3). Plasmid MT940 consists of the pMB1 origin of replication, kan resistance, pyr5 gene, upstream and downstream regions of the Cbh1 encoding gene from T. thermophilus for homologous recombination, and lacZ for blue/white screening. The plasmid contains the cbh2 promotor sequence from bases 3077-4649, the phytase including a signal sequence from bases 4650-5965 and the cbh1 terminator sequence from bases 5970-6176.

The plasmid was digested with Swal to remove the vector backbone and the fragment containing the phytase expression cassette was isolated from an agarose gel. Only the isolated DNA fragment was later used for transformation.

Expression Construct with Egl5 Promoter

A synthetic gene (GeneArt, ThermoFisher Scientific Inc., USA) (SEQ ID NO: 1) encoding a synthetic phytase from bacterial origin (disclosed in WO 2012/143862; as phytase PhV-99; SEQ ID NO. 2) was used for the construction of a phytase expression plasmids. For the secretion of the phytase, a signal sequence encoding for a signal peptide derived from T. thermophilus was added to the mature sequence of the phytase. A promotor sequence amplified from the up-stream region of the Egl5 encoding gene (MYCTH: XP_003659323.1) and a terminator sequence amplified from the downstream region of the Cbh1 encoding gene from T. thermophilus were used as regulatory elements to drive the expression of the phytase. Using standard cloning techniques, the expression plasmid pMT2195 (SEQ ID NO: 5) was constructed based on the E. coli standard cloning vector MT940 (SEQ ID NO: 3). Plasmid MT940 consists of the pMB1 origin of replication, kan resistance, pyr5 gene, upstream and downstream regions of the Cbh1 encoding gene from T. thermophilus for homologous recombination, and lacZ for blue/white screening. The plasmid contains the egl5 promotor sequence from bases 3077-4252, the phytase including a signal sequence from bases 4253-5568 and the cbh1 terminator sequence from bases 5573-5779.

The plasmid was digested with Swal to remove the vector backbone and the fragment containing the phytase expression cassette was isolated from an agarose gel. Only the isolated DNA fragment was later used for transformation.

Expression Construct with Cbh1 Promoter

A synthetic gene (GeneArt, ThermoFisher Scientific Inc., USA) (SEQ ID NO: 1) encoding a synthetic phytase from bacterial origin (disclosed in WO 2012/143862; as phytase PhV-99; SEQ ID NO. 2) was used for the construction of a phytase expression plasmids. For the secretion of the phytase, a signal sequence encoding for a signal peptide derived from T. thermophilus was added to the mature sequence of the phytase. A promotor sequence amplified from the up-stream region of the Cbh1 encoding gene (MYCTH: XP_003660789.1) and a terminator sequence amplified from the downstream region of the Cbh1 encoding gene from T. thermophilus were used as regulatory elements to drive the expression of the phytase. Using standard cloning techniques, the expression plasmid pMT2198 (SEQ ID NO: 6) was constructed based on the E. coli standard cloning vector MT940 (SEQ ID NO: 3). Plasmid MT940 consists of the pMB1 origin of replication, kan resistance, pyr5 gene, upstream and downstream regions of the Cbh1 encoding gene from T. thermophilus for homologous recombination, and lacZ for blue/white screening. The plasmid contains the cbh1 promotor sequence from bases 3077-4559, the phytase including a signal sequence from bases 4560-5875 and the cbh1 terminator sequence from bases 5880-6086.

The plasmid was digested with Swal to remove the vector backbone and the fragment containing the phytase expression cassette was isolated from an agarose gel. Only the isolated DNA fragment was later used for transformation.

Expression Construct with Cbh3 Promoter

A synthetic gene (GeneArt, ThermoFisher Scientific Inc., USA) (SEQ ID NO: 1) encoding a synthetic phytase from bacterial origin (disclosed in WO 2012/143862; as phytase PhV-99; SEQ ID NO. 2) was used for the construction of a phytase expression plasmids. For the secretion of the phytase, a signal sequence encoding for a signal peptide derived from T. thermophilus was added to the mature sequence of the phytase. A promotor sequence amplified from the up-stream region of the Cbh3 encoding gene (MYCTH: XP_003666507.1) and a terminator sequence amplified from the downstream region of the Cbh1 encoding gene from T. thermophilus were used as regulatory elements to drive the expression of the phytase. Using standard cloning techniques, the expression plasmid pMT2443 (SEQ ID NO: 7) was constructed based on the E. coli standard cloning vector MT940 (SEQ ID NO: 3). Plasmid MT940 consists of the pMB1 origin of replication, kan resistance, pyr5 gene, upstream and downstream regions of the Cbh1 encoding gene from T. thermophilus for homologous recombination, and lacZ for blue/white screening. The plasmid contains the cbh3 promotor sequence from bases 3077-5225, the phytase including a signal sequence from bases 5226-6541 and the cbh1 terminator sequence from bases 6546-6752.

The plasmid was digested with Swal to remove the vector backbone and the fragment containing the phytase expression cassette was isolated from an agarose gel. Only the isolated DNA fragment was later used for transformation.

### Example 3

Generation of Phytase Producing T. thermophilus Strains

For the expression of a phytase, the T. thermophilus host strain UV18 #100f Δpyr5 Δalp1 Δku70 (construction described in detail in WO 2017/093450) from the C1 lineage, a strain with uracil auxotrophy, reduced protease activity, and impaired non-homologous end joining (NHEJ) repair system, was transformed as described in example 1 with the Swal-digested and isolated phytase (s. example 2) expression constructs from plasmids pMT2194 (SEQ ID NO: 4), pMT2195 (SEQ ID NO: 5), pMT2198 (SEQ ID NO: 6) or pMT2443 (SEQ ID NO: 7). The transformants were incubated for 3-6 days at 37° C. on enriched minimal medium for pyr5 selection to select for restored uracil prototrophy by complementing the pyr5 deletion with the pyr5 marker as known in the art. Colonies were re-streaked and checked for the integration of the phytase expression cassette using PCR with primer pairs specific for the phytase expression cassette and the cbh1 locus as known in the art. A transformant tested positive for the phytase expression construct at the cbh1 locus was selected for further characterization.

### Example 4

### Phytase Activity Assay

The phytase activity is determined in microtiter plates. The phytase containing supernatant is diluted in reaction buffer (250 mM Na-acetate, 1 mM CaCl₂, 0.01% Tween 20, pH 5.5) such that the measurement stays within the linear range of the assay. 10 μl of the enzyme solution are incubated with 140 μl substrate solution (6 mM Na-phytate (Sigma P3168) in reaction buffer) for 1 h at 37° C. The reaction is quenched by adding 150 μl of trichloroacetic acid solution (15% w/w). To detect the liberated phosphate, 20 μl of the quenched reaction solution are treated with 280 μl of freshly made-up color reagent (60 mM L-ascorbic acid (Sigma A7506), 2.2 mM ammonium molybdate tetrahydrate, 325 mM H₂SO₄), and incubated for 20 min at 37° C., and the absorption at 820 nm is subsequently determined. For the blank value, the substrate buffer on its own is incubated at 37° C. and the 10 μl of enzyme sample are only added after quenching with trichloroacetic acid. The color reaction is performed analogously to the remaining measurements. The amount of liberated phosphate is determined via a calibration curve of the color reaction with a phosphate solution of known concentration.

### Example 5

### Analysis of Phytase Production in Small-Scale Cultivation

Generated mutant strains were fermented in small-scale cultivation and the supernatants were analyzed. T. thermophilus strains grown on agar were inoculated in 1 ml cultivation medium as shown in Table 1 in a 96-deepwell microtiter plate. The strains were fermented at 37° C. on a microtiter plate shaker at 900 rpm and 80% humidity for 72 hours. 300 μl of the 72-hour-preculture were transferred in 700 μl cultivation medium as shown in Table 2 in a 96-deepwell microtiter plate. The strains were fermented at 37° C. on a microtiter plate shaker at 900 rpm and 80% humidity for 96 hours.

Cell-free supernatants were harvested at the end of cultivation and subjected to a phytase activity assay (s. example 4).

| | Pcbh3 | Pcbh3 | Pegl5 | Pegl5 | Pcbh2 | Pcbh2 | Pcbh1 | Pcbh1 |
|---|---|---|---|---|---|---|---|---|
| | (1.6 kb) | (2.1 kb) | (1.2 kb) | (1.3 kb) | (1.6 kb) | (2.4 kb) | (1.5 kb) | (2.0 kb) |
| % | 130.9 | 170.7 | 87.4 | 100.0 | 79.3 | 125.3 | 97.0 | 89.8 |

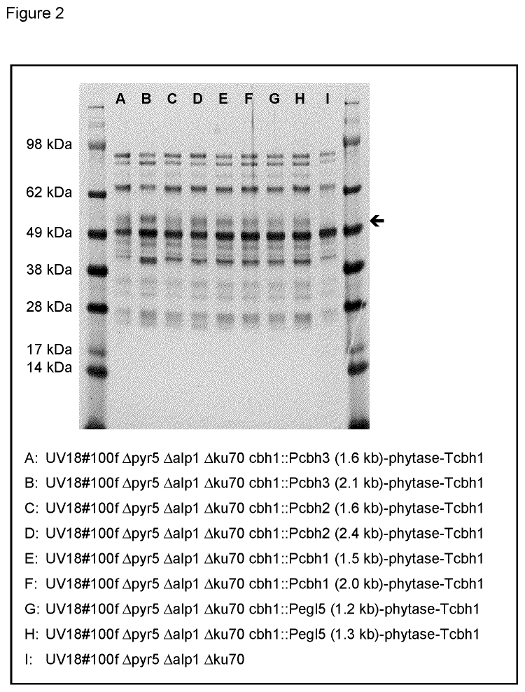

The supernatants were analyzed by SDS-PAGE. The gel was loaded in all cases with equal amounts of protein, as determined by measuring the protein concentration, and stained with Coomassie Blue (FIG. 2). In contrast to the parental strain UV18 #100f Δpyr5 Δalp1 Δku70, a protein band of the glycosylated phytase at approx. 55 kDa can clearly be seen in the different phytase production strains. The band is most prominent in the strain UV18 #100f Δpyr5 Δalp1 Δku70 cbh1::Pcbh3 (2.1 kb)-phytase-Tcbh1 that overexpresses the phytase gene driven by the cbh3 (2.1 kb) promoter.

TABLE 1
Cultivation medium

| | | | |
|---|---|---|---|
| Glucose | 10 | g/l | |
| Mg₂SO₄*7H₂O | 0.49 | g/l | |
| K₂SO₄ | 1.21 | g/l | |
| CaSO₄*2H₂O | 0.47 | g/l | |
| KH₂PO₄ | 1.75 | g/l | |
| (NH₄)₂SO₄ | 4.63 | g/l | |
| CuSO₄*5H₂O | 1.46 | mg/l | |
| FeSO₄*7H₂O | 4.56 | mg/l | |
| ZnSO₄*7H₂O | 20.05 | mg/l | |
| MnSO₄*H₂O | 3.92 | mg/l | |
| Na₂MoO₄*2H₂O | 1.37 | mg/l | |
| EDTA | 50 | mg/l | |
| Biotin | 0.006 | mg/l | |
| Penicillin | 50000 | U/l | |
| Streptomycin | 50 | mg/l | |
| Casamino acids | 1 | g/l | |
| MES | 21.33 | g/l | set pH to 6.0 |

TABLE 2
Cultivation medium

| | | | |
|---|---|---|---|
| Glucose | 14.29 | g/l | |
| Mg₂SO₄*7H₂O | 0.65 | g/l | |
| K₂SO₄ | 1.63 | g/l | |
| CaSO₄*2H₂O | 0.63 | g/l | |
| KH₂PO₄ | 2.35 | g/l | |
| (NH₄)₂SO₄ | 9.31 | g/l | |
| CuSO₄*5H₂O | 2.09 | mg/l | |
| FeSO₄*7H₂O | 6.51 | mg/l | |
| ZnSO₄*7H₂O | 28.64 | mg/l | |
| MnSO₄*H₂O | 5.60 | mg/l | |
| Na₂MoO₄*2H₂O | 1.96 | mg/l | |
| EDTA | 71.43 | mg/l | |
| Biotin | 0.009 | mg/l | |
| Penicillin | 71429 | U/l | |
| Streptomycin | 71.43 | mg/l | |
| MES | 60.93 | g/l | set pH to 6.75 |

1. An isolated promoter capable of conferring expression in a microorganism of the phylum Ascomycota comprising a sequence selected from the group consisting of:
(i) a sequence set forth in SEQ ID NO: 16 or 17; and
(ii) a sequence having at least 85% sequence identity over its entire length to a sequence of (i); 16 or 17 and;
(iii) a sequence that hybridizes under high stringency conditions comprising hybridisation at 42° C. in 1×SSC and 50% formamide, followed by washing at 65° C. in 0.3×SSC, to a sequence of (i) or (ii) or a complementary sequence thereto; and
(iv) a fragment of at least continuous 1000 bp of a sequence of (i) to (iii) wherein the promoters comprising a sequence as defined in (i) to (iv) are conferring expression in a microorganism of the phylum Ascomycota.

2. An expression construct comprising an isolated promoter according to claim 1 operably linked to a heterologous nucleic acid.

3. An expression vector comprising an isolated promoter according to claim 1.

4. A method for producing an expression construct, said method comprising operably linking an isolated promoter according to claim 1 to a heterologous nucleic acid.

5. A process for producing a vector, said method comprising linking an isolated promoter according to claim 1 to a vector.

6. (canceled)

7. (canceled)

8. A transgenic microorganism of the phylum Ascomycota comprising an expression construct according to claim 2.

9. The transgenic microorganism according to claim 8 wherein the expression construct is integrated into the genome of said microorganism.

10. A method for producing a transgenic microorganism of the phylum Ascomycota, said method comprising introducing an isolated promoter according to claim 1 into said microorganism.

11. The expression construct according to claim 2, wherein the operably linked heterologous nucleic acid encodes a protein selected from the group consisting of hydrolase, isomerase, ligase, lyase, oxidoreductase or transferase.

12. A method for expression of a heterologous nucleic acid in a recombinant microorganism of the phylum Ascomycota, comprising the step of cultivating said recombinant microorganism in a medium conducive for the expression of said heterologous nucleic acid, wherein the microorganism comprises a promoter according to claim 1 operably linked to the heterologous nucleic acid.

13. A method for the production of a target protein in a recombinant microorganism of the phylum Ascomycota, comprising the steps of
(i) cultivating a recombinant microorganism of the phylum Ascomycota in a medium conducive for the expression of said protein, wherein the microorganism comprises a promoter according to claim 1 operably linked to a heterologous nucleic acid encoding said target protein, and
(ii) recovering said target protein.
